(19) United States
(12) Patent Application Publication
(10) Pub. No.: US 2025/0166777 A1
(43) Pub. Date: May 22, 2025

(54) PREDICTING AND DETERMINING EFFICACY OF A LUNG CANCER THERAPY IN A PATIENT

(71) Applicant: CLEARNOTE HEALTH, INC., San Diego, CA (US)

(72) Inventors: Samuel Levy, San Diego, CA (US); Gulfem Dilek Guler, San Diego, CA (US); Yuhong Ning, San Diego, CA (US); David Haan, San Diego, CA (US)

(21) Appl. No.: 18/965,801

(22) Filed: Dec. 2, 2024

Related U.S. Application Data
(63) Continuation of application No. PCT/US2023/024376, filed on Jun. 2, 2023.
(60) Provisional application No. 63/348,325, filed on Jun. 2, 2022.

(51) Int. Cl. G16H 20/17; C12Q 1/6806; C12Q 1/6809; C12Q 1/6869; G16B 20/10; G16B 30/00; G16B 40/20; G16H 10/60; G16H 50/70

(52) U.S. Cl. CPC G16H 20/17; C12Q 1/6806; C12Q 1/6809; C12Q 1/6869; G16B 20/10; G16B 30/00; G16B 40/20; G16H 10/60; G16H 50/70; C12Q 2600/106; C12Q 2600/154

(57) ABSTRACT

Disclosed herein are methods for monitoring a lung cancer patient during lung cancer therapy to determine whether the patient is responding to the therapy, and for predicting whether a lung cancer patient, prior to beginning lung cancer therapy, is likely to respond to the therapy. The methods involve generation and analysis of hydroxymethylation signatures, wherein, in monitoring efficacy, a patient 5hmC signature obtained during therapy is compared to a baseline 5hmC signature, while in predicting efficacy, the patient 5hmC signature is compared to a reference 5hmC profile. Analysis of 5hmC levels at certain hydroxymethylation biomarker loci indicate whether the patient is likely to benefit from or continue benefitting from a particular lung cancer therapy. The invention also provides a method for ascertaining whether a lung cancer patient is responding to a lung cancer therapy, wherein a 5hmC molecular response score MR_{5hmc} is calculated from analysis of 5hmC levels at selected 5hmC biomarker loci, with a positive value generally indicating that the patient is responding to the therapy. Data sets comprising biomarker loci that are differentially hydroxymethylated with respect to therapy response or nonresponse are also provided.

### TECHNICAL FIELD

The present invention relates generally to the treatment of cancer, and more particularly relates to the management of patients receiving a lung cancer therapy such as an immunotherapy. The invention provides methods for predicting the efficacy of treating a lung cancer patient with a particular therapy and for monitoring the response of a lung cancer patient receiving a therapy.

### BACKGROUND

Methods that enable monitoring response to cancer therapy are crucial to enable informed decisions on whether or not to continue a cancer therapy, to spare a patient from unnecessary exposure to potentially multiple side effects and to provide the most effective treatment as early as possible in the course of the disease. Immunotherapy, in particular, presents a challenge in detecting a patient's response to therapy early, as when monitored with conventional imaging methods, tumors may appear as though increasing in size, when in fact they are enlarged due to positive drug action (such as may be due to an immune response), a phenomenon known as pseudo-progression. Monitoring immunotherapy response to detect potential resistance is important to do as early as possible, so that administration of an ineffective immunotherapy can be stopped (preventing any immunotherapy-associated adverse events) and thereby enable switching to another therapy quickly. Effective methods for predicting and monitoring a cancer patient's response to therapy, particularly immunotherapy, are sorely needed.

One of the ways in which others have attempted to address this problem is through identifying and monitoring tumor-specific mutations in ctDNA. However, this approach requires knowledge of tumor mutational status a priori and suffers from low levels of ctDNA and genomic (mutational) plasticity observed in patients.

Lung cancer is the most common cancer worldwide and is also the leading cause of cancer-related deaths globally, estimated to claim 1.8 million lives annually; see Sung et al. (2021), “Global Cancer Statistics 2020: GLOBOCAN Estimates of Incidence and Mortality Worldwide for 36 Cancers in 185 Countries,” Ca Cancer J Clin 71, 209-249. Most lung cancer patients are diagnosed with advanced disease, and 85% to 90% of all lung cancers cases are characterized as non-small cell lung cancer (NSCLC). Among the treatment options available for NSCLC, immunotherapy has cemented its place in the management of advanced stage disease, achieving better patient outcomes in both first line setting and beyond, as a monotherapy or combination therapy. See Wang et al. (2021), “Toward personalized treatment approaches for non-small-cell lung cancer,” Nat Med 27, 1345-1356; and Shields et al. (2021), “Immunotherapy for Advanced Non-Small Cell Lung Cancer: A Decade of Progress,” Am Soc Clin Oncol Educ Book 41, e105-e127.

In the treatment of cancers using an immunotherapy approach, immune checkpoint inhibitors (ICIs) targeting programmed cell death protein 1 (PD-1) and programmed cell death protein ligand 1 (PD-L1) have achieved remarkable success. ICIs work by reversing tumor microenvironment-induced T cell inhibition and thereby restore anti-tumor immunity.

While ICIs can elicit effective and durable responses, however, only a subset of patients show any benefit. Measurement of PD-L1 expression in tumors was initially adopted in the clinic, early on, as a patient selection biomarker for anti-PD1. However, PD-L1 expression frequently fails to identify those patients who could benefit from ICI treatment. This has been demonstrated, for example, by non-responsiveness seen in PD-L1-expressing patients as well as survival statistics that do not correlate with PD-L1 expression (See Brahmer et al. (2015), “Nivolumab versus Docetaxel in Advanced Squamous-Cell Non-Small-Cell Lung Cancer,” New Engl J Medicine 373:123-135; and Rodriguez-Abreu et al. (2021), “Pemetrexed plus platinum with or without pembrolizumab in patients with previously untreated metastatic nonsquamous NSCLC: protocol-specified final analysis,” KEYNOTE-189 Ann Oncol 32:881-895). Additional biomarkers that have emerged to improve patient selection for ICI therapy include assessment of tumor-infiltrating lymphocytes (Tumeh et al. (2014), “PD-1 blockade induces responses by inhibiting adaptive immune resistance,” Nature 515:568-571); T-cell-inflamed gene-expression profile (Ayers et al. (2017), “IFN-γ-related mRNA profile predicts clinical response to PD-1 blockade,” J. Clin. Invest. 127:2930-2940; Cristescu et al. (2018), “Pan-tumor genomic biomarkers for PD-1 checkpoint blockade-based immunotherapy,” Science 362: eaar3593), and tumor mutational burden (TMB) Rizvi et al. (2015), “Mutational landscape determines sensitivity to PD-1 blockade in non-small-cell lung cancer,” Science 348:124-128). Combinatorial use of such biomarkers serve as molecular readouts for the various components of the cancer-immunity cycle (Chen et al. (2013), “Oncology Meets Immunology: The Cancer-Immunity Cycle,” Immunity 39:1-10), thereby providing mechanistic insights that can indeed enrich for ICI-responding patients. However, the current array of biomarkers not only falls short in accurately identifying ICI responders, but also presents practical challenges associated with respect to the availability of sufficient tumor biopsy material, tumor heterogeneity, and the technical complexities involved in the standardization of assay analysis and interpretation. Combinations of multiple biomarker assays to improve accuracy of predicting responders also increases process complexity and cost. Furthermore, the requirement for invasive tumor biopsy in patients can prove challenging for immunotherapy response monitoring. Therefore, non-invasive and robust biomarkers that can improve ICI patient selection and response monitoring have yet to be established.

Liquid biopsy approaches utilizing plasma-derived cell-free DNA (cfDNA) present key advantages for biomarker discovery and development in ICI response prediction and monitoring setting. Circulating cell-free DNA is composed of DNA fragments found in the blood that originate from genomes of dying cells of different tissues and blood cells, reflecting cell turnover under normal/healthy conditions as well as altered homeostasis caused by diseases (Rostami et al. (2020), “Senescence, Necrosis, and Apoptosis Govern Circulating Cell-free DNA Release Kinetics,” Cell Rep. 31 (13): 107830); and Barefoot et al., “Detection of Cell Types Contributing to Cancer from Circulating, Cell-Free Methylated DNA,” Front. Genet. (27 Jul. 2021)). Unlike traditional tissue biopsies that can only sample locally and at accessible tumor sites, plasma-derived cfDNA can capture tumor heterogeneity with circulating tumor DNA originating from single as well as multiple tumor sites. Liquid biopsies also provide access to cfDNA originating from the tumor microenvironment and immune cells. Moreover, non-invasive approaches like liquid biopsies allow collection of serial samples over time, enabling dynamic monitoring of treatment response and resistance as well as disease recurrence.

Epigenetic modifications in cfDNA, such as 5-methylcytosine (5mC) and 5-hydroxymethylcytosine (5hmC), have been widely investigated in liquid biopsy-based diagnostic approaches. 5hmC is a stable epigenetic mark that originates from oxidation of 5mC by the ten-eleven translocation (TET) family of dioxygenases. Unlike 5mC, 5hmC has been shown to generally mark transcriptionally permissive chromatin state and is positively correlated with transcriptional activation, particularly for tissue- and cell type-specific genes (REF: Cui, X.-L. et al. A human tissue map of 5-hydroxymethylcytosines exhibits tissue specificity through gene and enhancer modulation. Nat Commun 11, 6161 (2020). Szulwach, K. E. et al. Integrating 5-hydroxymethylcytosine into the epigenomic landscape of human embryonic stem cells. Plos Genet 7, (2011). In the context of the immune system, dynamic changes in 5hmC profiles were observed to be important for immune cell differentiation and function; see Nestor et al. (2016), “5-Hydroxymethylcytosine Remodeling Precedes Lineage Specification during Differentiation of Human CD4+ T Cells,” Cell Rep. 16 (2): 559-570; and Tsiouplis et al. (2021), “TET-Mediated Epigenetic Regulation in Immune Cell Development and Disease,” Frontiers Cell Dev. Biology 8:623948. As such, 5hmC signatures in plasma-derived cfDNA provide a rich medium for biomarker discovery for various applications spanning from early detection to treatment selection and response monitoring.

There remains an unmet and pressing need in the art for improved methods for predicting a lung cancer patient's response to immunotherapy and monitoring a lung cancer patient's response during immunotherapy. An ideal method would be reliable and non-invasive, using cell-free DNA analysis in conjunction with predictive hydroxymethylation biomarkers.

### SUMMARY OF THE INVENTION

Tumor and normal cell DNA is released into the bloodstream, and a cell-free DNA (cfDNA) sample extracted therefrom can be analyzed with respect to genetic and epigenetic signatures. As alluded to above, epigenetic signatures include, by way of example, DNA methylation and DNA hydroxymethylation, with 5hmC profile (or “hydroxymethylome” or “hydroxymethylation signature”) of particular interest herein.

The present invention is predicated on the discovery of a set of hydroxymethylation biomarkers that, optionally in combination with one or more other types of biomarkers, features, and/or patient-specific characteristics, correlates with the likelihood that a lung cancer patient is responding to or will respond to treatment with a particular lung cancer therapy, e.g., an immunotherapy. A “biomarker” as that term is used herein refers to a characteristic that can be measured as an indicator of normal biological processes, pathogenic processes, or responses to an exposure or intervention, including therapeutic interventions.

In a first embodiment, the invention provides a method for monitoring a patient with a lung cancer during lung cancer therapy to determine efficacy of the therapy, the method comprising:
(a) obtaining a baseline hydroxymethylation signature for a lung cancer patient prior to receiving a lung cancer therapy by: (i) obtaining a cell-free DNA (cfDNA) sample from the patient, enriching for hydroxymethylated DNA in the sample, amplifying the hydroxymethylated DNA, and sequencing the amplified hydroxymethylated DNA in a manner that identifies 5-hydroxymethylcytosine (5hmC)-containing fragments or sites in the DNA; and (ii) measuring hydroxymethylation levels in the sequenced cfDNA at each of a plurality of hydroxymethylation biomarker loci, wherein each hydroxymethylation biomarker locus is selected as exhibiting an increase or decrease in hydroxymethylation in a manner that correlates with the presence of lung cancer;
(b) using the baseline hydroxymethylation signature as a first input parameter to a computer-generated predictive model comprising a trained machine learning model, thereby providing a first probability score;
(c) obtaining a monitoring hydroxymethylation signature for the lung cancer patient by repeating the process of (a) during treatment of the patient with the lung cancer therapy;
(d) using the monitoring hydroxymethylation signature as a second input parameter to the computer-generated predictive model to provide a second probability score; and
(e) comparing the second probability score to the first probability score to derive a differential probability score characterizing a likelihood that the patient is responding to the lung cancer therapy.

Each biomarker locus in the aforementioned method is selected as exhibiting an increase or decrease in hydroxymethylation in a manner that correlates with lung cancer tumor load.

The baseline probability score and the probability score obtained during therapy (the “monitoring probability score”) are typically calculated using a logistic regression analysis of the differences in hydroxymethylation level at each of the hydroxymethylation biomarker loci. Alternatives to logistic regression are also envisioned, including traditional statistical methods, as will be known to those in the field.

The hydroxymethylation biomarker loci are selected as exhibiting differential hydroxymethylation with regard to lung cancer tumor load, as may be established, for instance, by a Wilcoxon rank-sum test with a p-value of less than 0.05 and a fold change (“FC”) of at least 1.5 between lung cancer patients who do not respond to the lung cancer therapy and lung cancer patients who do respond to the lung cancer therapy.

In one aspect, step (e) further comprises combining the differential probability score with an additional feature value for at least one additional feature type to characterize the likelihood that the patient is responding to the lung cancer therapy. Additional feature types include DNA fragment size distribution, copy number variation (CNV), cfDNA concentration, methylation profile, T-cell-inflamed gene expression profile, circulating tumor DNA count, serum CA19-9 level, serum CA125 level, LAG3 expression, IDO-1 expression, T-cell count, inflammation gene signature, myeloid-derived suppressor cell count, lymphocyte count, deficient mismatch repair, tumor mutational burden, presence or absence of germline mutations, a patient-specific clinical parameter, and combinations of any of the foregoing. Combining the additional feature value with the hydroxymethylation information is generally done using a stacked ensemble analysis.

In some embodiments, the lung cancer is non-small cell lung cancer.

In other embodiments, the lung cancer is selected from adenocarcinomas, squamous cell carcinomas, small-cell lung carcinomas, adenosquamous carcinomas, carcinoid tumors, bronchial gland carcinomas, and sarcomatoid carcinomas.

In some embodiments, the lung cancer therapy is an immunotherapy.

The method may further comprise obtaining an additional patient hydroxymethylation signature later in the course of the lung cancer therapy and comparing the additional hydroxymethylation signature to the baseline signature, the monitoring signature, or both, to calculate a probability score that the patient is continuing to respond to the therapy.

By “responding positively to lung cancer therapy,” “responding to lung cancer therapy,” “responding positively to immunotherapy” or simply “responding to immunotherapy” is meant that the lung cancer patient treated with the lung cancer therapy exhibits a Complete Response (CR), a Partial Response (PR), or Stable Disease (SD) for a period of at least six months, as defined in the RECIST 1.1 guidelines as set forth in Eisenhauer et al. (2009), “New response evaluation criteria in solid tumours: Revised RECIST guideline (version 1.1),” European J. Cancer 45 (2): 228-247), the disclosure of which is incorporated by reference herein. Lung cancer patients that exhibit Progressive Disease (PD), as that term is also defined in the RECIST 1.1 guidelines, are deemed nonresponders to immunotherapy.

In another embodiment, the invention provides a method for determining a likelihood that a lung cancer patient will respond to treatment with a selected lung cancer therapy, where the method comprises:
(a) obtaining a hydroxymethylation signature for a lung cancer patient by: (i) obtaining a cell-free DNA (cfDNA) sample from the patient, enriching for hydroxymethylated DNA in the sample, amplifying the hydroxymethylated DNA, and sequencing the amplified hydroxymethylated DNA in a manner that identifies 5-hydroxymethylcytosine (5hmC)-containing fragments or sites in the DNA;
(b) mapping the sequenced hydroxymethylated DNA to each of a plurality of hydroxymethylation biomarker loci in a reference hydroxymethylation profile comprising a composite of hydroxymethylation signatures for a population group of individuals who have at least one shared characteristic selected from having lung cancer and responding to a lung cancer therapy and having lung cancer and not responding to the lung cancer therapy;
(c) determining differences in extent and location between the patient hydroxymethylation signature and the reference hydroxymethylation profile at each locus; and
(d) using the extent and location of the differences, calculating a probability score representing the likelihood that the lung cancer patient will respond to treatment with a lung cancer therapy.

Each hydroxymethylation signature in the composite that serves as the reference hydroxymethylation profile comprises a hydroxymethylation level at each of the plurality of hydroxymethylation biomarker loci, and, as before, each hydroxymethylation biomarker locus is selected as exhibiting an increase or decrease in hydroxymethylation in a manner that correlates with lung cancer tumor load.

In some embodiments, the plurality of hydroxymethylation biomarker loci are associated with T-cell inflamed genes. The genes, in some embodiments, include genes selected from CXCR6, TIGIT, CD27, PDCD1LG2, CD274, CD8A, LAG3, NKG7, CCL5, CMKLR1, PSMB10, CXCL9, IDO1, HLA-DQA1, CD276, STAT1, HLA-DRB1, CD276, STAT1, HLA-DRB1, and HLA-E.

In some embodiments, the plurality of hydroxymethylation biomarker loci in the reference hydroxymethylation profile are selected from those in the tables of FIGS. 29-38.

In some embodiments, the plurality of hydroxymethylation biomarker loci in the reference hydroxymethylation profile are selected from those in the tables of FIGS. 36-38.

As with the previous method, prediction of response to a lung cancer therapy can combine an additional feature value for at least one additional feature type to characterize the likelihood that the patient will respond to the lung cancer therapy.

If a lung cancer therapy is determined to be potentially useful in treating the patient, or is deemed effective in treating a patient already undergoing the therapy—i.e., because the probability score exceeds a predefined threshold—the patient can begin or continue the lung cancer therapy. If the analysis indicates that the patient is unlikely to respond to treatment with a particular lung cancer therapy, a decision to pursue a different course of action can be made. The patient is thereby spared unnecessary treatment, potential adverse events resulting from immunotherapy, and loss of valuable time during the course of the disease.

As may be deduced from the above, the selected loci that serve as hydroxymethylation biomarkers herein comprise loci selected for their relevance to responsiveness to treatment with a lung cancer therapy such as an immunotherapy. By “relevance” is meant that a hydroxymethylation biomarker locus, alone or in combination with one or more other hydroxymethylation biomarker loci, tends to exhibit an increase or decrease in hydroxymethylation in a manner that correlates with the treatment responsiveness of the lung cancer patient, with regard to, for example, tumor size, stage, invasiveness, grade, and the like. In general, relevance can be determined by assessing the correlation between a potential biomarker and the likelihood that the lung cancer patient will respond to treatment with a lung cancer therapy such as an immunotherapy. That correlation, as briefly alluded to above, generally, although not necessarily, involves (a) a fold change of at least 1.5 between the hydroxymethylation level at a particular locus in a responding subject and the hydroxymethylation level at the same locus for a nonresponding subject, and (b) differential hydroxymethylation between responders and nonresponders with a p-value of less than 0.05 as determined by the Wilcoxon rank-sum test (Mann et al. (1947), “On a Test of Whether one of Two Random Variables is Stochastically Larger than the Other,” Annals of Mathematical Statistics 18 (1): 50-60).

The reference hydroxymethylation profile is a data set representing the hydroxymethylation level of each of a plurality of hydroxymethylation biomarker loci, where the data set is a composite of hydroxymethylation signatures of a plurality of individuals having at least one shared characteristic. The reference profile may be a composite of hydroxymethylation signatures for lung cancer patients who have responded to treatment with a particular lung cancer therapy, or it may be a composite of hydroxymethylation signatures for lung cancer patients who are nonresponders to treatment with a particular lung cancer therapy.

The reference profile may also be a focused reference profile that can enhance the accuracy of the evaluation method. Different types of focused reference hydroxymethylation profiles may be constructed from different population groups, and an appropriate reference profile can then be selected for the evaluation of a particular patient. To assess a female lung cancer patient in her seventies, for example, a narrowed, or focused, reference profile would be used that is generated from a set of female lung cancer patients, lung cancer patients aged 70 to 80, or female lung cancer patients aged 70-80 who have responded positively to treatment with the lung cancer therapy of interest. It will be appreciated that additional such focused reference profiles can be constructed depending on the attributes of the lung cancer patient undergoing evaluation or monitoring.

The methods of the present invention provide an improvement over currently available methods of evaluating the likelihood that a subject with lung cancer is responding to or will respond to treatment with a lung cancer therapy such as immunotherapy, insofar as those methods are largely based on clinical symptoms and radiographic evaluation, or on biomarkers that are not sufficiently predictive.

In additional embodiments, then, the invention provides methods for:
Determining a course of treatment for a subject with lung cancer;
Determining whether an immunotherapy used to treat a subject with lung cancer should be discontinued;
Determining an alternative course of treatment after an initial lung cancer therapy is discontinued; and
Reducing the risk that a subject with lung cancer who is unlikely to positively respond to immunotherapy will receive immunotherapy.

In another embodiment, the invention provides a data set for use in a lung cancer therapy response analysis, the data set comprising a composite of hydroxymethylation signatures of a plurality of individuals who have at least one shared characteristic selected from having lung cancer and responding to a lung cancer therapy and having lung cancer and not responding to the lung cancer therapy, wherein each hydroxymethylation signature in the composite comprises a hydroxymethylation level at each of a plurality of hydroxymethylation biomarker loci selected from those in the tables of FIGS. 29-38.

In some embodiments, the plurality of hydroxymethylation biomarker loci are selected from those in the tables of FIGS. 36-38.

The invention also provides a method for ascertaining whether a lung cancer patient is responding to a lung cancer therapy by calculating a 5hmC molecular response score MR_{5hmc} from analysis of 5hmC levels at selected 5hmC biomarker loci, with a positive value generally indicating that the patient is responding to the therapy and a negative value generally indicating that the patient is a nonresponder.

Accordingly, in a further embodiment, a method is provided for identifying differentially hydroxymethylated sites for use as hydroxymethylation biomarkers in evaluating whether a lung cancer patient is a responder or a nonresponder to a lung cancer therapy, wherein the method comprises:
(a) obtaining cfDNA from each of a plurality of lung cancer patients who are known responders or known nonresponders to the therapy;
(b) determining a baseline count T₀ in CPM at each of a plurality of candidate hydroxymethylation biomarker loci in the cfDNA obtained from each of the patients;
(c) determining a later count T_{R} in CPM after beginning the therapy and confirming response or nonresponse to the therapy, wherein T_{R} is determined for each of the plurality of candidate hydroxymethylation biomarker loci in the cfDNA obtained from each of the patients;
(d) selecting as hydroxymethylation biomarker loci those candidate hydroxymethylation biomarker loci exhibiting a threshold p-value of less than 0.05 and a difference ζ of at least 1.5, wherein

$$ζ = \{ {T}_{R} - {T}_{0} ) / {T}_{0} .$$

In some aspects of the embodiment, the method further includes calculating a value for

log 2(T_{R}/T₀)
at each of the selected hydroxymethylated biomarker loci, and identifying the calculated values as xᵢ at each locus i or yⱼ at each locus j, wherein the xᵢ and yⱼ are positively and negatively correlated with treatment response, respectively.

In another embodiment, the invention provides a method for determining whether a lung cancer patient is responding to a lung cancer therapy, the method comprising:
(a) in a cfDNA sample obtained from the patient, determining a baseline count T₀ at each of the hydroxymethylation biomarker loci selected according to the above method for identifying differentially hydroxymethylated sites suitable as hydroxymethylation biomarkers in evaluating whether a lung cancer patient is a responder or a nonresponder to the lung cancer therapy;
(b) in a later cfDNA sample obtained from the patient, determining a later count T_{Q} at each of the selected hydroxymethylation biomarker loci after beginning the therapy;
(c) calculating a value for

log 2(T_{Q}/T₀)
at each of the selected hydroxymethylated biomarker loci, and identifying the calculated values as xᵢ at each locus i or yⱼ at each locus j, wherein the xᵢ and yⱼ are positively and negatively correlated with treatment response, respectively;
(d) calculating a 5hmC molecular response score (MR_{5hmc}) for the patient using the equation

$${MR}_{5 hmC} = {μ}_{x} - {μ}_{y}$$

wherein μₓ is the mean of the xᵢ over i loci and μ_{y} is the mean of the yⱼ over j loci; and
(e) determining that the patient is responding to the therapy when the 5hmC molecular response score is positive.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 schematically illustrates the timing of blood draws and anti-PD1 dosing, as described in the Example.

FIG. 2 is a table indicating the clinical characteristics of the patient cohort.

FIG. 3 depicts the 5hmC landscape per sample as barplots showing the number of 5hmC peaks observed (top panel) and the number of genes/promoters that are hydroxymethylated (bottom panel).

FIG. 4 illustrates 5hmC peak overlap with gene and other genomic annotations.

FIG. 5 is a barplot showing 5hmC peak enrichment analysis (y-axis) over gene and other genomic annotations as identified in the x-axis.

FIG. 6 is a volcano plot showing differential 5hmC analysis comparing plasma-derived 5hmC profiles prior to treatment in patients who responded with patients who did not respond to anti-PD-1 therapy.

FIG. 7 provides the results of the gene set enrichment analysis (GSEA) using 5hmC counts over genes to compare baseline 5hmC profiles in responding vs non-responding patients, and reveals inflammatory and immune response enrichment in responders at baseline. Red indicates enrichment in responders. Blue indicates enrichment in non-responders.

FIG. 8 illustrates differential 5hmC over immune modulating genes associated with anti-PD-1 response.

FIG. 9 are boxplots showing mean baseline 5hmC level (FPKM) over T-cell inflamed genes (n=18) in responding (CRPR, meaning complete response or partial response, as defined previously coral) and non-responding (PD, progressive disease, teal) patients. P-value is calculated by Wilcoxon rank sum test.

FIG. 10 depicts Kaplan-Meier curves of overall survival in a cohort with a three-year follow up (n=19), where patients were stratified according to median 5hmC RPKM value over T-cell inflamed genes.

FIGS. 11 and 12 are volcano plots showing differential 5hmC analysis comparing plasma-derived 5hmC profiles at time of radiological response (TR) and at baseline (T0) in responders (FIG. 11) and in non-responders (FIG. 12).

FIG. 13 shows the overlap of genes with differential 5hmC counts, as identified by comparison of on-treatment samples to matched pre-treatment samples in responders and non-responders.

FIG. 14 shows the 5hmC fold change over the genes identified as exhibiting differential hydroxymethylation in both responders and non-responders.

FIGS. 15 and 16 are GSEA results obtained using 5hmC counts over genes to compare baseline to time of response in responding (FIG. 15) and non-responding (FIG. 16) patients, revealing several pathways associated with activation of immune response.

FIGS. 17 and 18 are heatmaps showing 5hmC fragment counts (log 2CPM) over top differentially hydroxymethylated regions (“DhMRs”) identified in responding (FIG. 13) and in non-responding (FIG. 14) patients, as determined by thresholding with p-value<0.05 and log FC>1.5 DhMR loci.

FIGS. 19 and 20 are tSNE plots showing segregation of lung cancer tissue samples from normal lung tissue controls using top DhMRs identified in plasma samples from responders (FIG. 19) and non-responders (FIG. 20).

FIGS. 21 and 22 are heatmaps showing 5hmC fragment counts (log 2CPM) in non-responding (FIG. 21) and responding (FIG. 22) patients over responder and non-responder top DhMRs, respectively.

FIG. 23 is a heatmap showing anti-PD-1 treatment-induced log fold change in 5hmC counts over 283 genomic loci in responder and non-responder cohorts.

FIG. 24 are genome browser screenshots over two distinct DhMRs with opposite behaviors between responding (coral) and non-responding (teal) patients as identified in FIG. 23. Peak 1 showing a decrease of 5hmC levels in a representative CRPR patient and an increase of 5hmC levels in a representative PD patient over time, where TO is baseline and T5 is time of response. Peak 2 shows opposite 5hmC changes between CRPR and PD.

FIG. 25 shows the 5hmC fold change over two example genomic loci in responding (coral) and non-responding (teal) patients over the course of treatment.

FIG. 26 is a spider plot showing overall 5hmC-based molecular response over all treatment-induced DhMRs as determined in FIG. 23 in responding (coral) and non-responding (teal) patients at each time point throughout treatment.

FIG. 27 is a graph illustrating the change in cancer prediction scores for responding (coral) and non-responding (teal) patients over the course of anti-PD-1 treatment. Each line represents a patient.

FIG. 28 is a boxplot showing the change in prediction scores comparing time of radiologic response (T_{R}) to baseline (T0).

FIG. 29 is a table showing the top differentially 5-hydroxymethylated regions upon treatment in responders for the subset of peaks in FIG. 6 with a fold change of greater than 1.5.

FIG. 30 is a table showing the top differentially 5-hydroxymethylated regions upon treatment in responders for the data represented in FIG. 11, with only those differentially hydroxymethylated regions included that exhibited a fold change of greater than 1.5 in at least eight responders.

FIG. 31 is an analogous table showing the top differentially 5-hydroxymethylated regions upon treatment in nonresponders for the data represented in FIG. 12, with only those differentially hydroxymethylated regions included that exhibited a fold change of greater than 1.5 in at least six nonresponders.

FIG. 32 is a table that indicates the top pathways with 5hmC accumulation according to gene set enrichment analysis (GSEA) of responders relative to non-responders prior to the start of treatment, using the data of FIG. 7. The top pathways were heavily immune-related such as allograft rejection, inflammatory response, and tumor necrosis factor alpha (TNFα) signaling via nuclear factor kB (NFkB) to bin responding patients.

FIG. 33 is a table indicating the top pathways of gene sets enriched at the time of response (TR) relative to the start of treatment (T0) in responders, determined from the data of FIG. 6. Among the top pathways were immune-related genesets such as interferon gamma (IFN-γ) response, inflammatory response, and interferon alpha response.

FIG. 34 is an analogous table indicating the top pathways of gene sets enriched at the time of response (TR) relative to the start of treatment (T0) in non-responders as determined from the data of FIG. 11.

FIG. 35 is a table providing a biomarker set corresponding to the genomic regions identified as having the most significant hydroxymethylation changes between the responding and non-responding cohorts, determined by calculating treatment-induced fold changes in 5hmC occupancy normalized to baseline ((TR−T0)/T0) and then identifying the regions with the most statistically significant changes by applying a threshold p-value of 0.05 and a fold change of at least 1.5 as shown in FIG. 12.

FIG. 36 is an analogous table showing the list of differentially hydroxymethylated genes with p-value below 0.05 as identified from comparison of plasma-derived 5hmC profiles prior to treatment in patients who responded with patients who did not respond to anti-PD-1 therapy as represented in FIG. 6.

FIGS. 37 and 38 are analogous tables showing the list of differentially hydroxymethylated genes with p-value below 0.05 as identified from comparison of plasma-derived 5hmC profiles at time of radiological response (T_{R}) and at baseline (TO) in responders (FIG. 37) and in non-responders (FIG. 38), as represented in FIG. 11 and FIG. 12, respectively.

### DETAILED DESCRIPTION OF THE INVENTION

### 1. Terminology and Overview

Unless defined otherwise, all technical and scientific terms used herein have the meaning commonly understood by one of ordinary skill in the art to which the invention pertains. Specific terminology of particular importance to the description of the present invention is defined below. Other relevant terminology is defined in International Patent Publication No. WO 2017/176630 to Quake et al. for “Noninvasive Diagnostics by Sequencing 5-Hydroxymethylated Cell-Free DNA.” The aforementioned patent publication as well as all other patent documents and publications referred to herein are expressly incorporated by reference.

In this specification and the appended claims, the singular forms “a,” “an” and “the” include plural referents unless the context clearly dictates otherwise. Thus, for example, “an adapter” refers not only to a single adapter but also to two or more adapters that may be the same or different, “a template molecule” refers to a single template molecule as well as a plurality of template molecules, and the like.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

The term “sample” as used herein relates to a material or mixture of materials, typically, although not necessarily, in liquid form, containing one or more analytes of interest.

The term “biological sample” as used herein relates to a sample derived from a biological fluid, cell, tissue, or organ of a human subject, comprising a mixture of biomolecules including proteins, peptides, lipids, nucleic acids, and the like. Generally, although not necessarily, the sample is a blood sample such as a whole blood sample, a serum sample, or a plasma sample.

A “nucleic acid sample” as that term is used herein refers to a biological sample comprising nucleic acids. The nucleic acid sample may be a cell-free nucleic acid sample that comprises nucleosomes, in which case the nucleic acid sample is sometimes referred to herein as a “nucleosome sample.” The nucleic acid sample may also be comprised of cell-free DNA wherein the sample is substantially free of histones and other proteins, such as will be the case following cell-free DNA purification. The nucleic acid samples herein may also contain cell-free RNA.

A “sample fraction” refers to a subset of an original biological sample, and may be a compositionally identical portion of the biological sample, as when a blood sample is divided into identical fractions. Alternatively, the sample fraction may be compositionally different, as will be the case when, for example, certain components of the biological sample are removed, with extraction of cell-free nucleic acids being one such example.

As used herein, the term “cell-free nucleic acid” encompasses both cell-free DNA and cell-free RNA, where the cell-free DNA and cell-free RNA may be in a cell-free fraction of a biological sample comprising a body fluid. The body fluid may be blood, including whole blood, serum, or plasma. In most instances, the biological sample is a blood sample, and a cell-free nucleic acid sample, e.g., a cell-free DNA sample, is extracted therefrom using now-conventional means known to those of ordinary skill in the art and/or described in the pertinent texts and literature; kits for carrying out cell-free nucleic acid extraction are commercially available (e.g., the AllPrep® DNA/RNA Mini Kit and QIAmp DNA Blood Mini Kit, both available from Qiagen, or the MagMAX Cell-Free Total Nucleic Acid Kit and the MagMAX DNA Isolation Kit, available from ThermoFisher Scientific). Also see, e.g., Hui et al. Fong et al. (2009) Clin. Chem. 55 (3): 587-598.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, and up to about 10,000 or more bases composed of nucleotides, e.g., deoxyribonucleotides or ribonucleotide. Nucleic acids may be produced enzymatically, chemically synthesized, or naturally obtained.

The terms “duplex” and “duplexed” are used interchangeably herein to describe two complementary polynucleotides that are base-paired, i.e., hybridized together. A DNA duplex is referred to herein as “double-stranded DNA” or “dsDNA” and may be an intact molecule or a molecular segment. For example, the dsDNA herein referred to as barcoded and adapter-ligated is an intact molecule, while the dsDNA formed between the nucleic acid tails of proximity probes in a proximity extension assay is a dsDNA segment.

The term “strand” as used herein refers to a single strand of a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, “positive” and “negative” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

“Adapters” as that term is used herein are short synthetic oligonucleotides that serve a specific purpose in a biological analysis. Adapters can be single-stranded or double-stranded, although the preferred adapters herein are double-stranded. In one embodiment, an adapter may be a hairpin adapter (i.e., one molecule that base pairs with itself to form a structure that has a double-stranded stem and a loop, where the 3′ and 5′ ends of the molecule ligate to the 5′ and 3′ ends of a double-stranded DNA molecule, respectively). In another embodiment, an adapter may be a Y-adapter. In another embodiment, an adapter may itself be composed of two distinct oligonucleotide molecules that are base paired with each other. As would be apparent, a ligatable end of an adapter may be designed to be compatible with overhangs made by cleavage by a restriction enzyme, or it may have blunt ends or a 5′ T overhang. The term “adapter” refers to double-stranded as well as single-stranded molecules. An adapter can be DNA or RNA, or a mixture of the two. An adapter containing RNA may be cleavable by RNase treatment or by alkaline hydrolysis. An adapter may be 15 to 100 bases, e.g., 50 to 70 bases, although adapters outside of this range are envisioned.

The term “adapter-ligated,” as used herein, refers to a nucleic acid that has been ligated to an adapter. The adapter can be ligated to a 5′ end and/or a 3′ end of a nucleic acid molecule. As used herein, the term “adding adapter sequences” refers to the act of adding an adapter sequence to the end of fragments in a sample. This may be done by filling in the ends of the fragments using a polymerase, adding an A tail, and then ligating an adapter comprising a T overhang onto the A-tailed fragments. Adapters are usually ligated to a DNA duplex using a ligase, while with RNA, adapters are covalently or otherwise attached to at least one end of a cDNA duplex preferably in the absence of a ligase.

The term “adapter-ligated sample”, as used herein, refers to a sample that has been ligated to an adapter. As would be understood given the definitions above, a sample that has been ligated to an asymmetric adapter contains strands that have non-complementary sequences at the 5′ and 3′ ends.

The term “amplifying” as used herein refers to generating one or more copies, or “amplicons,” of a template nucleic acid, such as may be carried out using any suitable nucleic acid amplification technique, such as technology, such as PCR, NASBA, TMA, and SDA.

The terms “enrich” and “enrichment” refer to a partial purification of template molecules that have a certain feature (e.g., nucleic acids that contain 5-hydroxymethylcytosine) from analytes that do not have the feature (e.g., nucleic acids that do not contain hydroxymethylcytosine). Enrichment typically increases the concentration of the analytes that have the feature by at least 2-fold, at least 5-fold or at least 10-fold relative to the analytes that do not have the feature. After enrichment, at least 10%, at least 20%, at least 50%, at least 80% or at least 90% of the analytes in a sample may have the feature used for enrichment. For example, at least 10%, at least 20%, at least 50%, at least 80% or at least 90% of the nucleic acid molecules in an enriched composition may contain a strand having one or more hydroxymethylcytosines that have been modified to contain a capture tag.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “next-generation sequencing” (NGS) or “high-throughput sequencing”, as used herein, refer to the so-called parallelized sequencing-by-synthesis or sequencing-by-ligation platforms currently employed by Illumina, Life Technologies, Roche, etc. Next-generation sequencing methods may also include nanopore sequencing methods such as that commercialized by Oxford Nanopore Technologies, electronic detection methods such as Ion Torrent technology commercialized by Life Technologies, and single-molecule fluorescence-based methods such as that commercialized by Pacific Biosciences.

The term “read” as used herein refers to the raw or processed output of sequencing systems, such as massively parallel sequencing. In some embodiments, the output of the methods described herein is reads. In some embodiments, these reads may need to be trimmed, filtered, and aligned, resulting in raw reads, trimmed reads, aligned reads.

A “Unique Feature Identifier” (UFI) sequence refers to a relatively short nucleic acid sequence that serves to identify a feature of a nucleic acid molecule. Nucleic acid template molecules and amplicons thereof that contain a UFI are sometimes referred to herein as “barcoded” template molecules or amplicons. Examples of UFI sequence types include, without limitation, the following:

A “source identifier sequence” (or “source UFI” or “source barcode”) identifies the biological sample (or other source) of origin. That is, each DNA molecule in a single sample is tagged with the same source identifier sequence, thus allowing the mixing of samples prior to sequencing. These UFIs may also be characterized as a “sample identifier sequence,” a “sample UFI,” or “sample barcode.”

A “fragment identifier sequence” (or “fragment UFI” or “fragment barcode”): In a nucleic acid sample in which nucleic acids have been fragmented, each fragment in a sample is barcoded with a corresponding fragment identifier sequence. Sequence reads that have non-overlapping fragment identifier sequences represent different original nucleic acid template molecules, while reads that have the same fragment identifier sequences, or substantially overlapping fragment identifier sequences, likely represent fragments of the same template molecule. The unique feature identified here is the template nucleic acid molecule from which a fragment derives.

A “strand identifier sequence” (or “strand UFI” or “strand barcode”) independently tags each of the two strands of a DNA duplex, so that the strand from which a read originates can be determined, i.e., as the W strand or the C strand.

A “5hmC identifier sequence” (or “5hmC barcode”) identifies DNA fragments originating from 5hmC-containing cell-free DNA template molecules in a sample, i.e., “hydroxymethylated” DNA.

A “5mC identifier sequence” (or “5mC barcode”) identifies DNA fragments originating from 5mC-containing cell-free DNA template molecules that do not contain 5hmC.

A “molecular UFI sequence” (or “molecular barcode”) is appended to every nucleic acid template molecule in a sample, and is random, such that, providing the UFI sequence is of sufficient length, every nucleic acid template molecule is attached to a unique UFI sequence. Molecular UFI sequences, as is known in the art, can be used to account for and offset amplification and sequencer errors, allow a user to track duplicates and remove them from downstream analysis, and enable molecular counting, and, in turn, the determination of an analyte concentration. See, e.g., Casbon et al. (2011) Nuc. Acids Res. 39 (12): 1-8. The “unique feature” here is the identity of the nucleic acid template molecules.

In some embodiments, a UFI may have a length in the range of from 1 to about 35 nucleotides, e.g., from 3 to 30 nucleotides, 4 to 25 nucleotides, or 6 to 20 nucleotides. In certain cases, the UFI may be error-detecting and/or error-correcting, meaning that even if there is an error (e.g., if the sequence of the molecular barcode is mis-synthesized, mis-read or distorted during any of the various processing steps leading up to the determination of the molecular barcode sequence) then the code can still be interpreted correctly. The use of error-correcting sequences is described in the literature (e.g., in U.S. Patent Publication Nos. U.S. 2010/0323348 to Hamati et al. and U.S. 2009/0105959 to Braverman et al., both of which are incorporated herein by reference).

The oligonucleotides that serve as UFI sequences herein may be incorporated into DNA molecule using any effective means, where “incorporated into” is used interchangeably herein with “added to” and “appended to,” insofar as the UFI can be provided at the end of a DNA molecule, near the end of a DNA molecule, or within a DNA molecule. For example, multiple UFIs can be end-ligated to DNA using a selected ligase, in which case only the final UFI is at the “end” of the molecule. In addition, in the proximity extension assay and histone modification methods described in detail infra, the UFI may be contained within the nucleic acid tail of a proximity probe, at the end of the nucleic acid tail of a proximity probe, or within the hybridized region generated upon the binding of probes to the protein target.

More generally, the term “detection” is used interchangeably with the terms “determining,” “measuring,” “evaluating,” “assessing,” “assaying,” and “analyzing,” to refer to any form of measurement, and include determining if an element is present or not. These terms include both quantitative and/or qualitative determinations. Assessing may be relative or absolute. “Assessing the presence of” thus includes determining the amount of a moiety present, as well as determining whether it is present or absent. Assessing the level at a hydroxymethylation biomarker locus refers to a determination of the degree of hydroxymethylation at that locus.

“Accuracy” refers to the degree of conformity of a measured or calculated quantity (a test reported value) to its accurate (or true) value. Clinical accuracy relates to the proportion of true outcomes (true positives (TP) or true negatives (TN) versus misclassified outcomes (false positives (FP) or false negatives (FN), and may be stated as a sensitivity, specificity, positive predictive values (PPV) or negative predictive values (NPV), or as a likelihood, or odds ratio, among other measures.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Clinical parameters” encompass all non-sample biomarkers of subject health status or other characteristics, such as, without limitation, lesion size; lesion location; patient age; patient weight; patient gender; patient ethnicity; family history; genetic mutations; and PD-L1 tumor staining result, which is currently used in the clinic to determine whether anti-PD-1 therapy is in order.

A “formula,” “algorithm,” or “model” is any mathematical equation, algorithmic, analytical or programmed process, or statistical technique that takes one or more continuous or categorical inputs and calculates an output value, sometimes referred to as a “probability score” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations.

Of particular use in combining hydroxymethylation levels at various biomarker loci and clinical parameters, optionally in further combination with other factors (e.g., non-hydroxymethylation biomarkers), are linear and non-linear equations and statistical classification analyses to determine the relationship between hydroxymethylation levels at the biomarker loci detected in a patient sample and the patient's likelihood of responding to an immunotherapy. In panel and combination construction, of particular interest are structural and syntactic statistical classification algorithms, and methods of risk index construction, utilizing pattern recognition and machine learning features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, and Hidden Markov Models, among others. Many such algorithmic techniques have been further implemented to perform both feature (loci) selection and regularization, such as in ridge regression, lasso, and elastic net, among others. Other techniques may be used in survival and time to event hazard analysis, including Cox, Weibull, Kaplan-Meier and Greenwood models well known to those of skill in the art. Many of these techniques are useful either combined with a hydroxymethylation biomarker selection technique, such as forward selection, backwards selection, or stepwise selection, complete enumeration of all potential biomarker sets, or panels, of a given size, genetic algorithms, or they may themselves include biomarker selection methodologies. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art.

“Likelihood,” in the context of one embodiment of the present invention, is the probability that a patient is responding or is not responding to treatment with a lung cancer therapy. In another embodiment, “likelihood” is the probability that a patient will respond or not respond to treatment with a particular lung cancer therapy.

A “hydroxymethylation level” refers to the extent of hydroxymethylation within a hydroxymethylation biomarker locus. The extent of hydroxymethylation is normally measured as hydroxymethylation density, e.g., the ratio of 5hmC residues to total cytosines, both modified and unmodified, within a nucleic acid region. Other measures of hydroxymethylation density are also possible, e.g., the ratio of 5hmC residues to total nucleotides in a nucleic acid region.

A “hydroxymethylation profile” or “hydroxymethylation signature” refers to a data set that comprises the hydroxymethylation level at each of a plurality of hydroxymethylation biomarker loci. The hydroxymethylation profile may be a reference hydroxymethylation profile that comprises composite a hydroxymethylation profile for a population of individuals with at least one shared characteristic, as explained elsewhere herein. The hydroxymethylation profile may also be a patient hydroxymethylation signature, constructed from the measurement of hydroxymethylation levels at each of a plurality of hydroxymethylation biomarker sites.

A “reference hydroxymethylation profile” thus refers to a data set representing the hydroxymethylation level of each of a plurality of hydroxymethylation biomarkers, where the data set is a composite of hydroxymethylation profiles of a plurality of individuals having at least one shared characteristic, e.g., lung cancer patients who respond to treatment with immunotherapy as determined by the RECIST 1.1 guidelines, or lung cancer patients who do not respond to treatment with immunotherapy as determined in the same manner.

The “hydroxymethylation biomarkers” herein comprise loci selected for their relevance to the likelihood that a lung cancer patient will respond to treatment with an immunotherapy.

The term “locus” as used throughout this application refers to a site on a nucleic acid molecule, wherein the nucleic acid molecule may be single-stranded or double-stranded, and further wherein an individual locus (or multiple “loci”) may be of any length, thus including a single CpG site as well as a full-length gene, or across larger features such as topologically associated domains, including when several such loci are aggregated into groups such as related sequence motifs, other homologies or functional characteristics (regardless of their adjacency or topological relationship). The loci herein may be contained within a gene body; within an annotation feature outside of the gene body, such as a promoter, an enhancer, a transcription initiation site, a transcription stop site, or a DNA binding site, or a combination thereof; or within an untranslated region, or “UTR” (including 3′UTRs and 5′UTRs).

It should be noted that some of the individual hydroxymethylation biomarkers disclosed herein may not have significant individual significance in the evaluation of a patient's responsiveness to immunotherapy, but when used in combination with one or more other types of biomarkers and, optionally, clinical parameters impacting on the evaluation and monitoring of a lung tumor, become significant in discriminating as a method of the invention requires, e.g., between a subject who is responding to immunotherapy and a subject who is not responding to immunotherapy.

For the purpose of this application, any two variables are considered to be “very highly correlated” when they have a Coefficient of Determination (R2) of 0.5 or greater. The present invention encompasses such functional and statistical equivalents to the presently disclosed hydroxymethylation biomarkers.

The term “correlate” as used herein in reference to two variables (e.g., two values, two sets of values, a value or value set and a disease state, a value or set of values and a risk associated with the disease state, or the like) indicates a tendency of the two variables to vary together. A “correlation” is a measure of the extent to which two or more variables fluctuate together. A positive correlation indicates the extent to which those variables increase or decrease in parallel. One example of a positive correlation is the relationship between a hydroxymethylation level at a hydroxymethylation biomarker locus, on the one hand, and the responsiveness of a lung cancer patient to an immunotherapy, on the other, when the hydroxymethylation level increases as the responsiveness of the subject increases. Conversely, a negative correlation would exist when the hydroxymethylation level at a hydroxymethylation biomarker locus decreases as a subject's responsiveness to immunotherapy decreases.

The present invention relates, in part, to the discovery that certain biological markers, particularly epigenetic markers relating to DNA hydroxymethylation, correlate with the likelihood that a subject with lung cancer will respond positively to a lung cancer therapy, such as an immunotherapy. The methods involve measuring the hydroxymethylation level at each of a plurality of hydroxymethylation biomarker loci to generate a hydroxymethylation signature for a patient, and then comparing the patient's hydroxymethylation signature to a reference hydroxymethylation profile, at each locus. The biomarkers are differentially hydroxymethylated with regard to likelihood of response to a particular lung cancer therapy, e.g., treatment with checkpoint blockade therapy for a patient with non-small-cell lung cancer, one of several types of “immunotherapies” as will be discussed elsewhere herein.

The invention also enables a practitioner to determine the effectiveness of a lung cancer therapy being administered to a subject with lung cancer; to diagnose lung cancer in a patient who has not yet had a lung tumor identified; to determine whether a lung tumor identified via imaging is a non-small-cell lung cancer; to assess the stage of an identified lung tumor; to predict whether a healthy individual is likely to develop lung cancer, particularly non-small-cell lung cancer; to identify the risk that an identified lung tumor will develop into cancer; and to identify a change in the size, stage, grade, or degree of invasiveness of a cancerous lung tumor.

The term “lung cancer” herein refers to any cancer of the lung, such as non-small-cell lung cancers including adenocarcinomas, squamous cell carcinomas, and large cell carcinomas; small-cell lung carcinomas; adenosquamous carcinomas; carcinoid tumors; bronchial gland carcinomas; and sarcomatoid carcinomas. Non-small cell lung cancer (NSCLC) is the most prevalent form of lung cancer, with adenocarcinomas most prevalent among non-smokers. The lung cancer patient who is evaluated using the present methods may be at an early or late stage of the disease, have a tumor that exhibits strong or weak PD-L1 staining, exhibit different spirometry results, and the like.

The “lung cancer patient” or simply “patient,” as those terms are used herein, refer to any living individual who has been diagnosed with lung cancer, via imaging, biopsy, or other known means, and refers to the intended recipient of an immunotherapy treatment as discussed in detail herein.

As used herein, the term “immunotherapy” refers to any method for treating disease by activating or suppressing the immune system. Examples of immunotherapies include, but are not limited to, cellular therapies such as dendritic cell therapy; antibody therapy; and cytokine therapy (for example, treatment with an interferon or an interleukin). Most commonly, the immunotherapy treatment discussed herein involves administration of a therapeutic antibody that binds to and blocks an immune checkpoint receptor protein such as CTLA-4, PD-1, or the like. PD-1 is a key immune checkpoint receptor, expressed by activated T cells and B cells, and mediates immunosuppression. Two cell surface glycoprotein ligands for PD-1 have been identified, Programmed Death Ligand-1 (PD-L1) and Programmed Death Ligand-2 (PD-L2), which are expressed on antigen-presenting cells as well as many human cancers and have been shown to down-regulate T cell activation and cytokine secretion upon binding to PD-1. Representative antibodies that target PD-1, a PD-1 ligand (e.g., PD-L1) or other immune checkpoint receptors or ligands thereof, and which are encompassed by the immunotherapies referenced herein include, without limitation, atezolizumab (Tecentriq®, Genentech); necitumumab (Portrazza®, Eli Lilly); nivolumab (Opdivo®, Bristol-myers Squibb); cemiplimab (Libtayo, Regeneron Pharmaceuticals); avelumab (Bavencio, EMD Serono): durvalumab (Imfinzi®, AstraZeneca); dostarlimab (Jemperli®, GlaxoSmithKline); retifanlimab (Zynyz®, Incyte); and pembrolizumab (Keytruda® Merck). It is to be understood that the invention is not limited in this respect, however, and that “immunotherapy” herein refers to any therapeutic treatment that activates or suppresses the immune system and is potentially useful in the treatment of lung cancer.

It should be noted that the methodology of the invention is not necessarily limited to lung cancer immunotherapy, but may extend to other lung cancer therapies as well.

### 2. Determination and Use of Baseline Hydroxymethylation Signature:

In a first embodiment, a method is provided for monitoring a patient with a lung cancer during lung cancer therapy to determine efficacy of the therapy. In the first step of this method, a baseline hydroxymethylation signature is obtained for the patient. The baseline hydroxymethylation signature comprises hydroxymethylation levels at each of a plurality of hydroxymethylation biomarker loci. The selected hydroxymethylation biomarker loci are differentially hydroxymethylated with respect to the likelihood that a lung cancer patient is responding to treatment with the selected lung cancer therapy, so that hydroxymethylation levels higher than or lower than those in in the baseline hydroxymethylation signature, obtained during treatment, correlate with the likelihood that the patient will respond or not respond to the immunotherapy treatment contemplated.

To generate the baseline hydroxymethylation signature for the patient, a cfDNA sample is obtained from the patient. Extraction of cfDNA from a blood sample can be carried out using any suitable technique, for example using the commercially available kits referenced in the preceding section. The cfDNA is then enriched, so that the concentration of the cfDNA is substantially increased, a virtual necessity because of the very low levels of cfDNA normally obtained. A generally preferred enrichment technique is described in International Patent Publication WO 2017/176630 to Quake et al., incorporated herein by reference in its entirety: an affinity tag is appended to 5hmC residues in a sample of cfDNA, and the tagged DNA molecules are then selectively removed by bonding to a functionalized solid support. An illustrative example of the method, as described in Quake et al., involves initially modifying end-blunted, adaptor-ligated double-stranded DNA fragments in the cell-free sample to covalently attach biotin, as the affinity tag, to 5hmC residues. This may be carried out by selectively glucosylating 5hmC residues with uridine diphospho (UDP) glucose functionalized at the 6-position with an azide moiety, a step that is followed by a spontaneous 1,3-cycloaddition reaction with alkyne-functionalized biotin via a “click chemistry” reaction. The DNA fragments containing the biotinylated 5hmC residues are adapter-ligated dsDNA template molecules that can then be pulled down with a solid support functionalized with a biotin-binding protein (e.g., avidin or streptavidin) in the enrichment step.

The captured cfDNA is then amplified without having been releasing from the support, resulting in a plurality of amplicons. Any suitable amplification technique may be employed (e.g., PCR, NASBA, TMA, SDA) although PCR is preferred.

Next, the patient cfDNA is sequenced in a manner that identifies 5hmC-containing fragments or sites in the cfDNA, as will be explained infra.

Then, hydroxymethylation levels in the sequenced cfDNA are measured at each of a plurality of hydroxymethylation biomarker loci, wherein each hydroxymethylation biomarker locus is selected as exhibiting an increase or decrease in hydroxymethylation in a manner that correlates with the presence of lung cancer or immunotherapy response.

The baseline hydroxymethylation signature obtained is used as a first input parameter to a computer-generated predictive model comprising a trained machine learning model, thereby providing a first probability score. Then, a second hydroxymethylation signature, also referred to herein as a “monitoring hydroxymethylation signature,” is determined for the lung cancer patient during treatment with a selected lung cancer therapy. This second hydroxymethylation signature is determined the same way as the baseline hydroxymethylation signature, discussed above. This second hydroxymethylation signature is used as a second input parameter to the computer-generated predictive model to provide a second probability score. Finally, the second probability score is compared to the first probability score to derive a differential probability score characterizing a likelihood that the patient is responding to the lung cancer therapy.

It may be that when the second probability score is greater than the first probability score, such that the differential probability score is positive, a determination can be made that the lung cancer therapy is likely ineffective, i.e., that the patient is not responding to the therapy. An alternative system may also be structured such that a negative differential probability score indicates a non-response to therapy.

In the embodiment wherein the lung cancer patient is not yet undergoing therapy, but a determination as to whether a particular therapy is likely to be effective in treating the patient, the baseline hydroxymethylation signature is compared to a reference hydroxymethylation profile instead of a hydroxymethylation signature obtained during the course of a therapy.

That is, the identified 5hmC residues are mapped to each of a plurality of loci in a reference hydroxymethylation profile, where each locus serves as a hydroxymethylation biomarker that, as noted above, is differentially hydroxymethylated with respect to the likelihood that a lung cancer patient will or will not respond to treatment with a particular lung cancer therapy. Information regarding hydroxymethylation levels is thus deduced from the sequence reads obtained. That is, the sequence reads are analyzed to provide a quantitative determination of which sequences are hydroxymethylated in the cfDNA, and the level of hydroxymethylation. This may be done by, e.g., counting sequence reads or, alternatively, counting the number of original starting molecules, prior to amplification, based on their fragmentation breakpoint and/or whether they contain the same molecular UFI. The use of molecular UFI sequences (or “molecular barcodes” as they are sometimes called) in conjunction with other features of the fragments (e.g., the end sequences of the fragments, which define the breakpoints) to distinguish between the fragments is known. See Casbon (2011) Nucl. Acids Res. 22 e81 and Fu et al. (2011) Proc. Natl. Acad. Sci. USA 108:9026-31), among others. Molecular barcodes are also described in U.S. Patent Publication Nos. 2015/0044687, 2015/0024950, and 2014/0227705, and in U.S. Pat. Nos. 8,835,358 and 7,537,897, as well as a variety of other publications.

A molecular UFI sequence is preferably incorporated into the adapters that are end-ligated to the cfDNA following extraction thereof. The adapters may be constructed so as to comprise an additional UFI sequence, e.g., a sample UFI sequence, a strand-identifier UFI sequence, or both.

Other methods of ascertaining the hydroxymethylation signature of DNA in the cell-free nucleic sample are described in International Patent Publication WO 2019/160994 A1 to Arensdorf et al. for “Methods for the Epigenetic Analysis of DNA, particularly Cell-Free DNA”; in co-pending U.S. patent application Ser. Nos. 16/275,237 and 17/118,234 to Arensdorf et al.; and in Liu et al. (2019) Nature Biotech. 37:424-29, all of which are incorporated by reference herein. These references are also useful in conjunction with an embodiment of the invention in which a patient's cfDNA methylation profile is identified in addition to the patient's cfDNA hydroxymethylation profile.

Mapping the identified 5hmC residues to each of a plurality of loci in a reference hydroxymethylation profile enables the determination of differences between the hydroxymethylation profile of the patient and the reference hydroxymethylation profile, with respect to both the extent and the location of those differences.

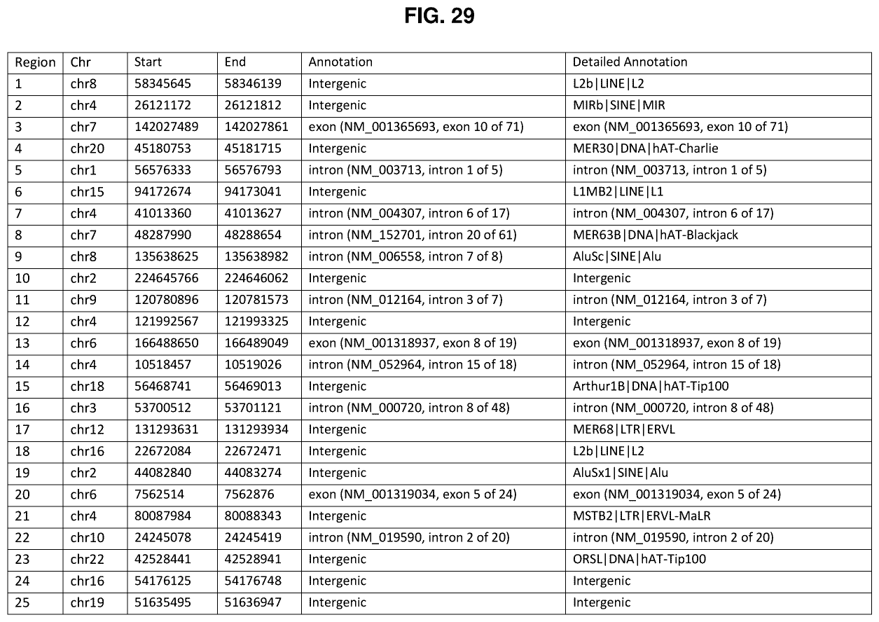

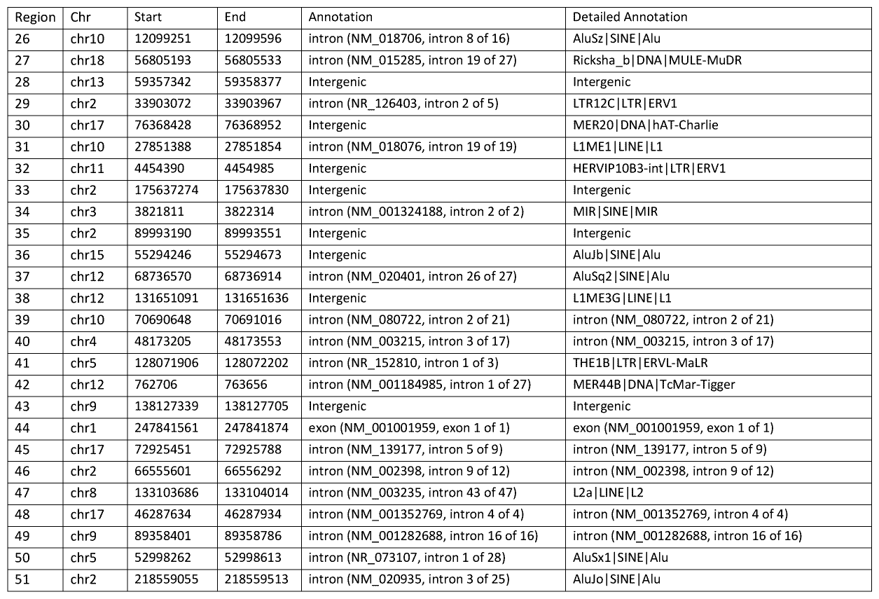

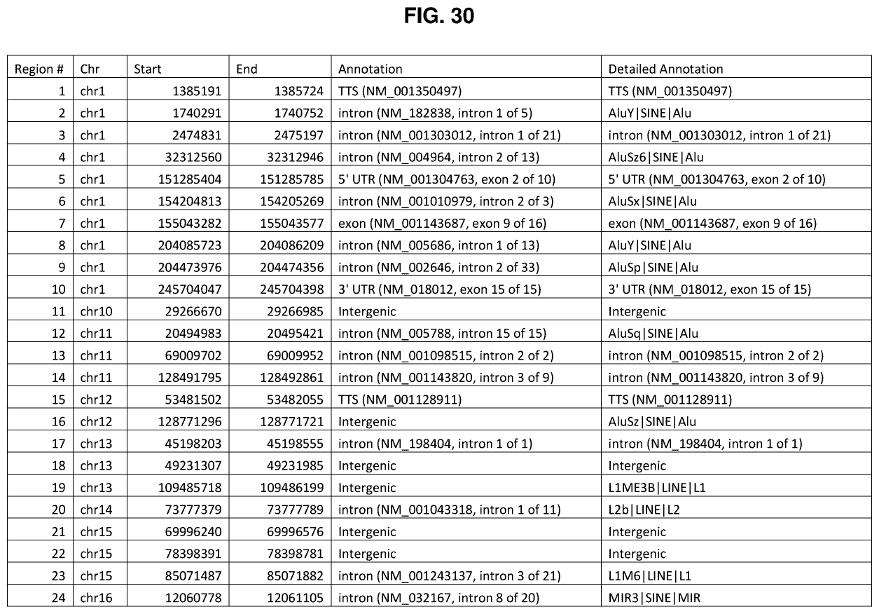

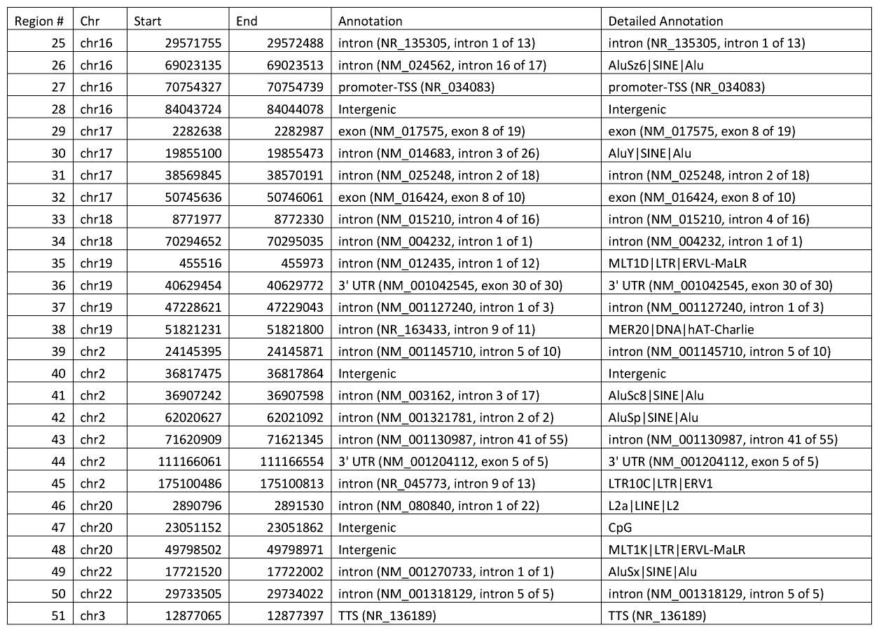

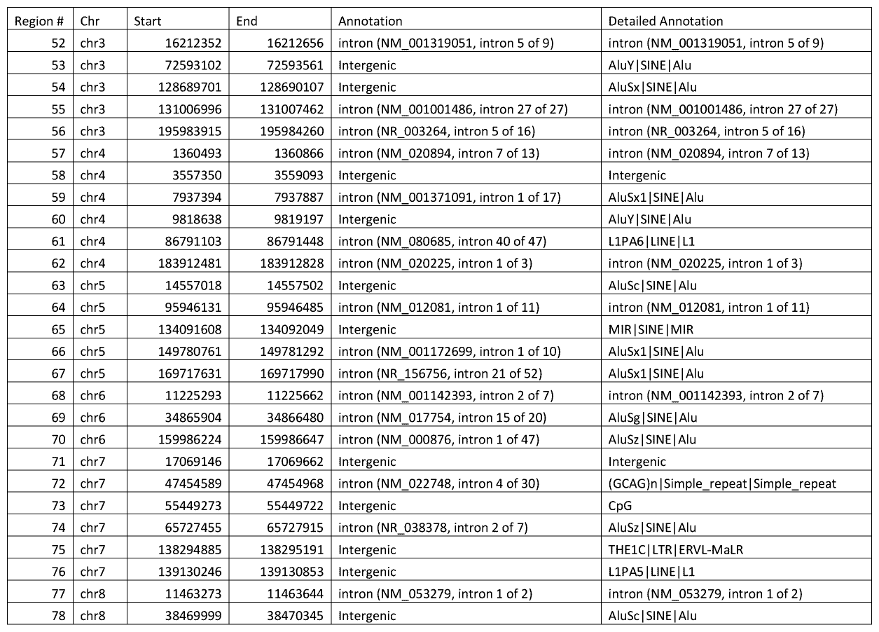

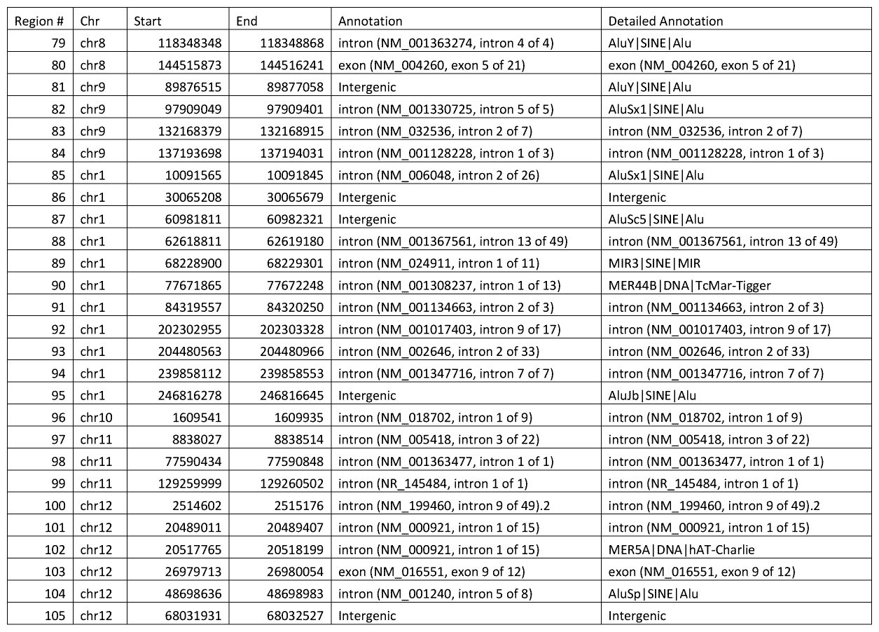

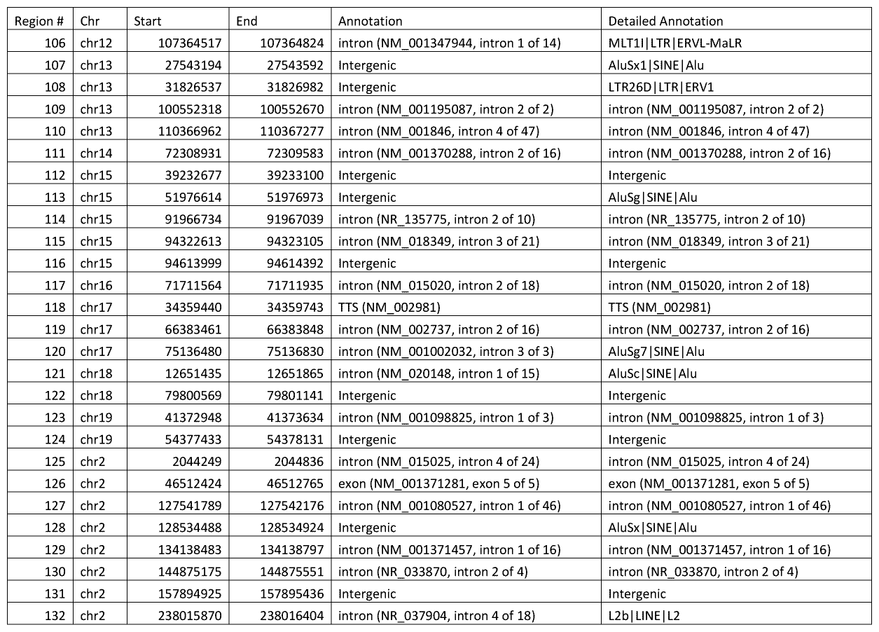

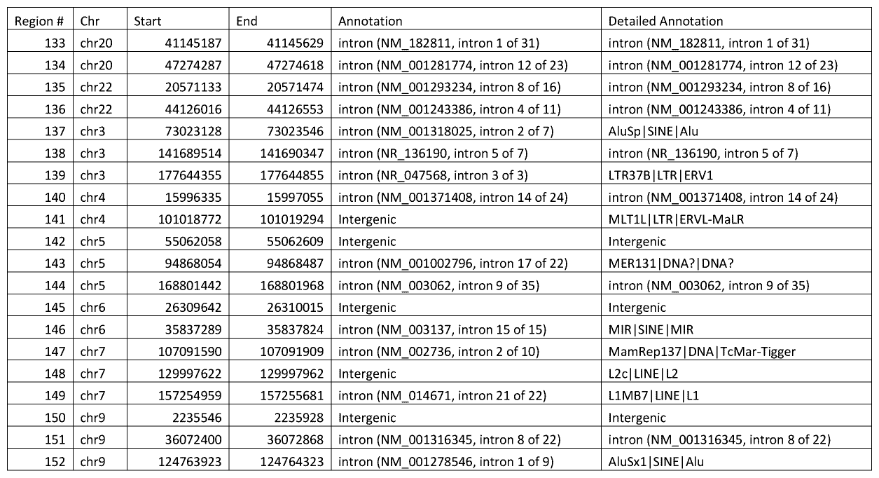

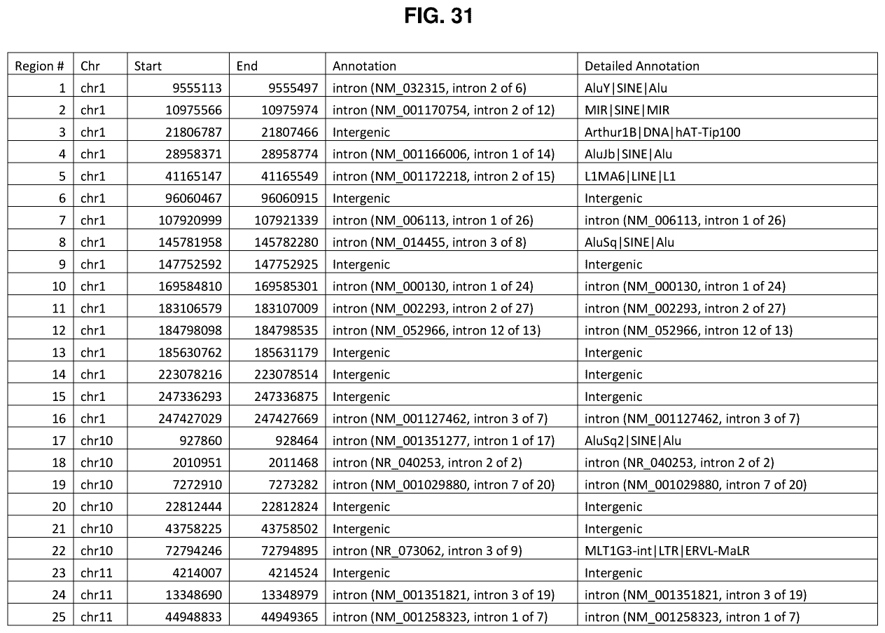

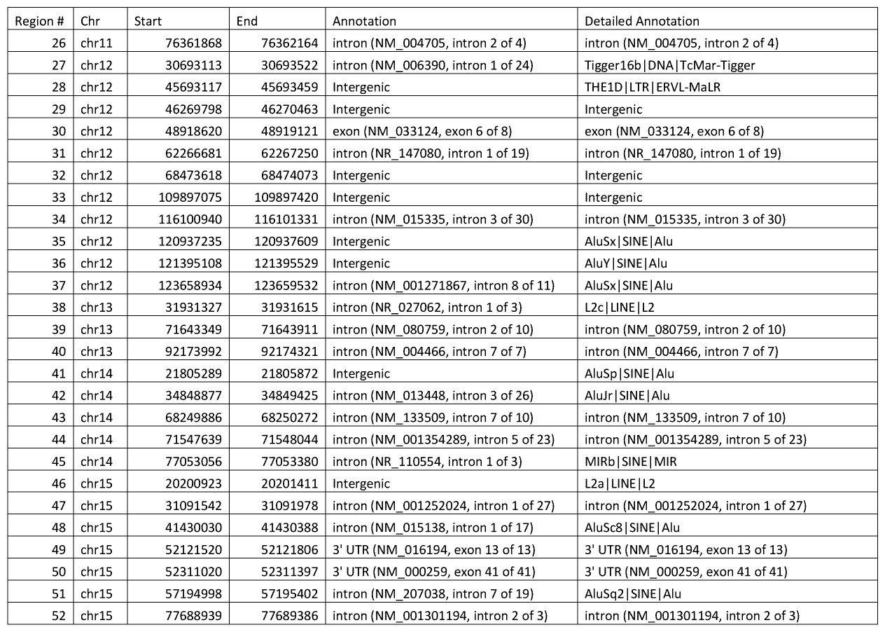

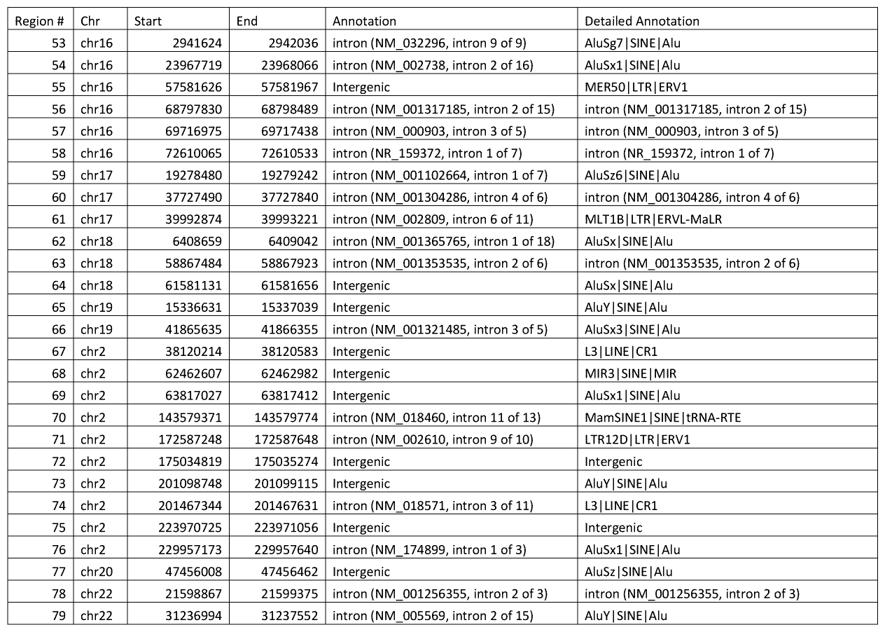

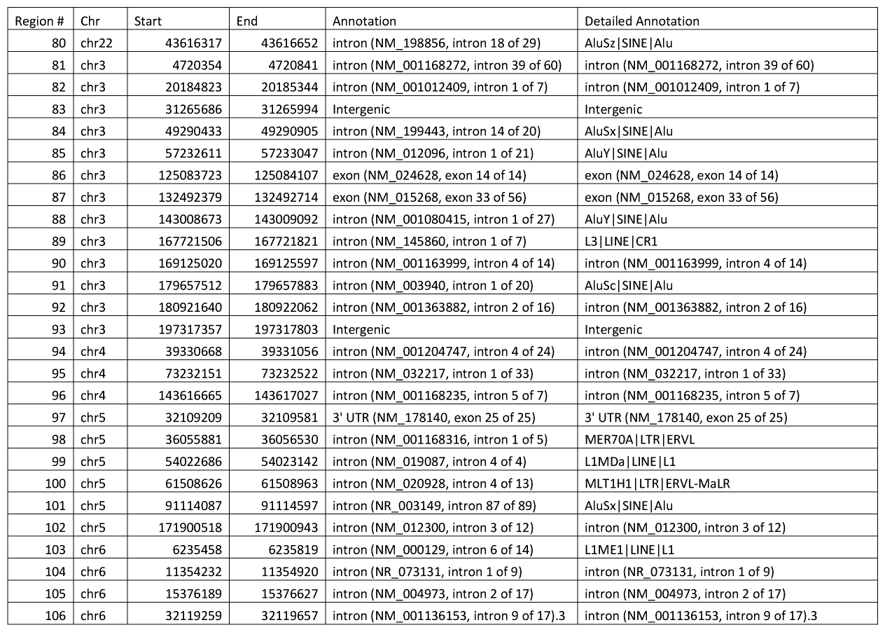

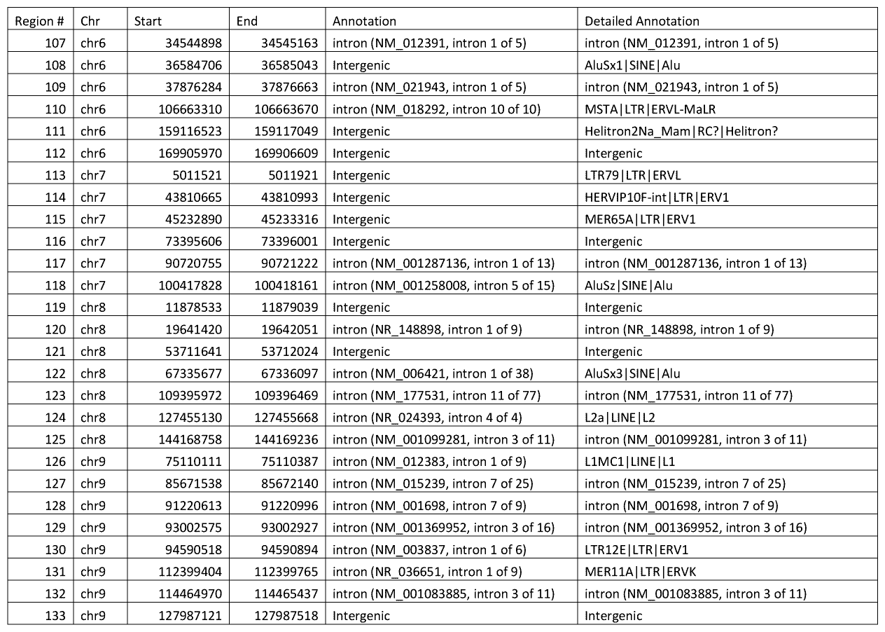

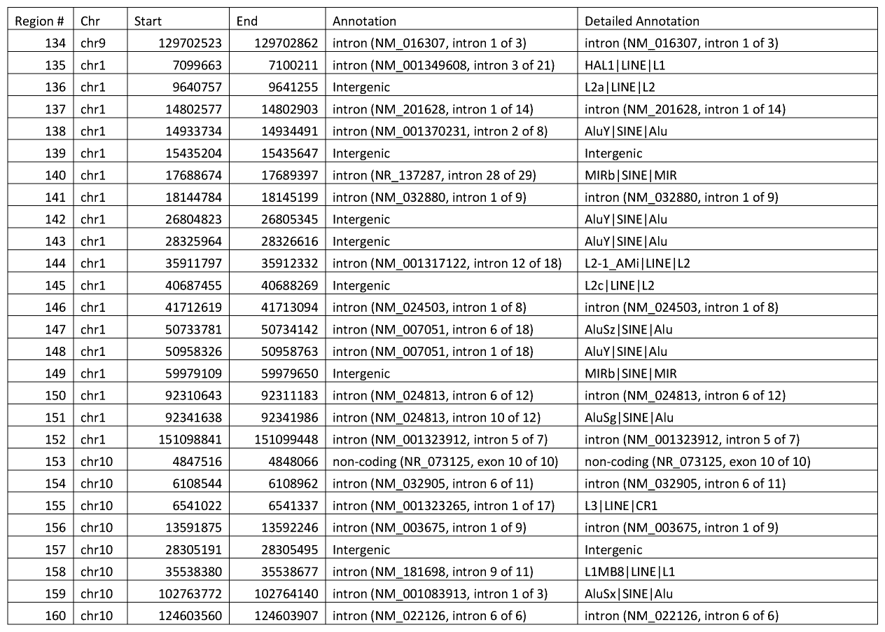

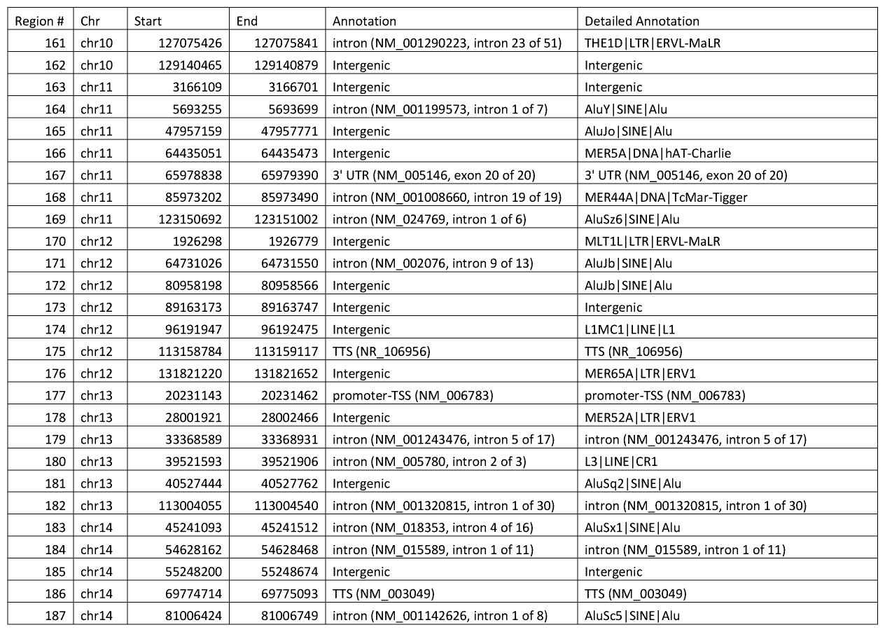

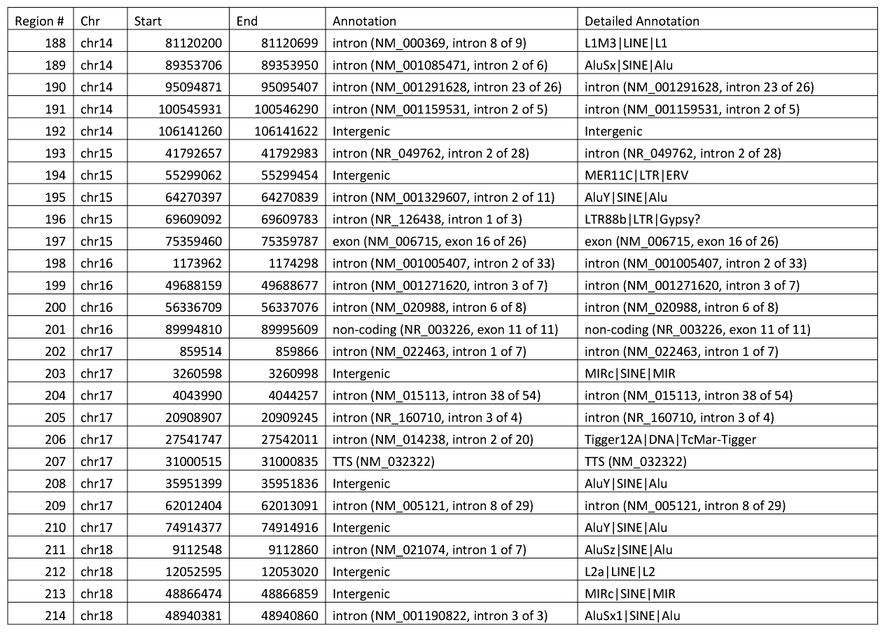

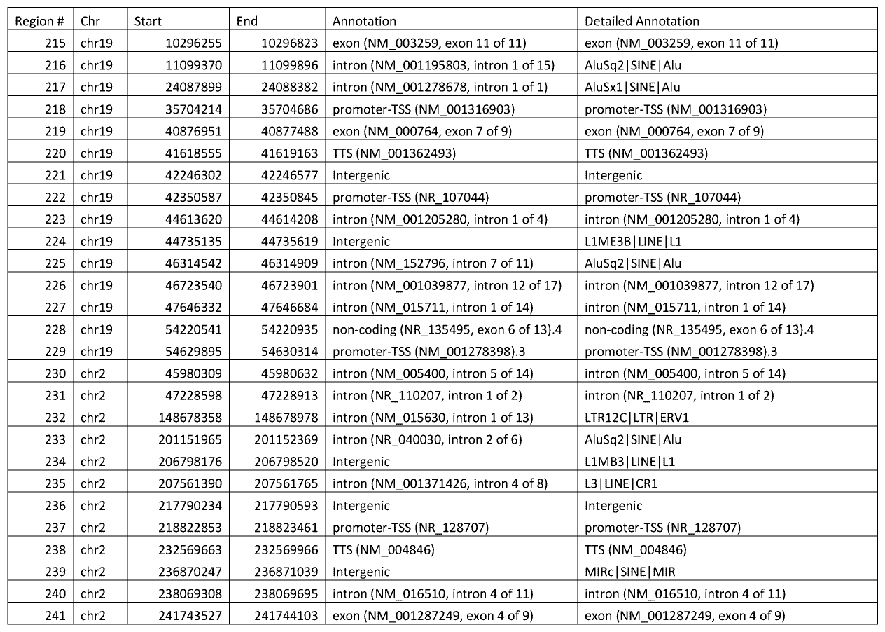

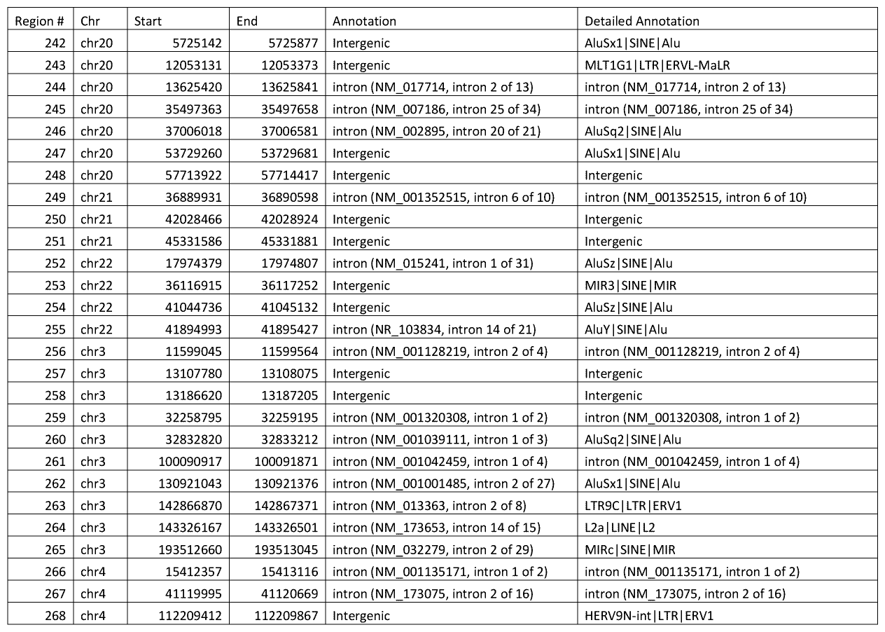

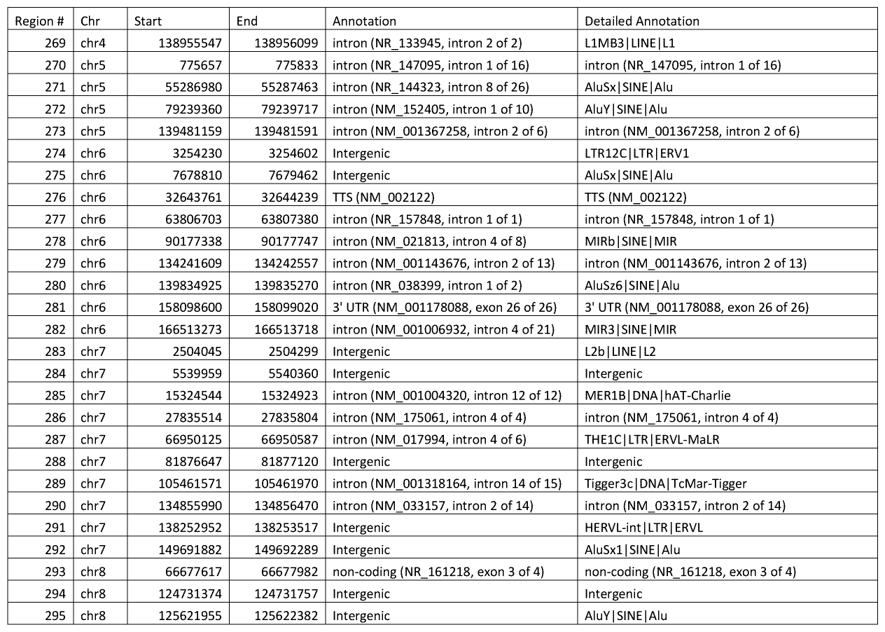

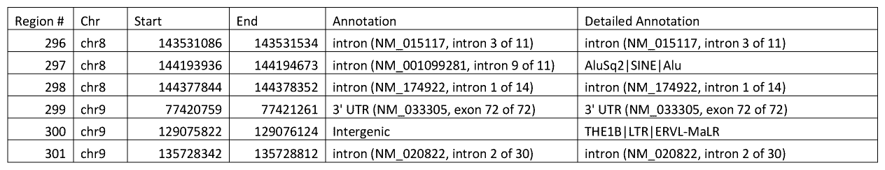

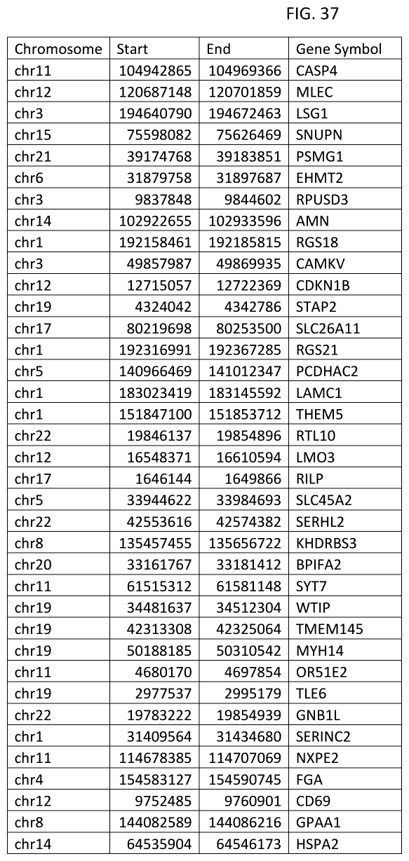

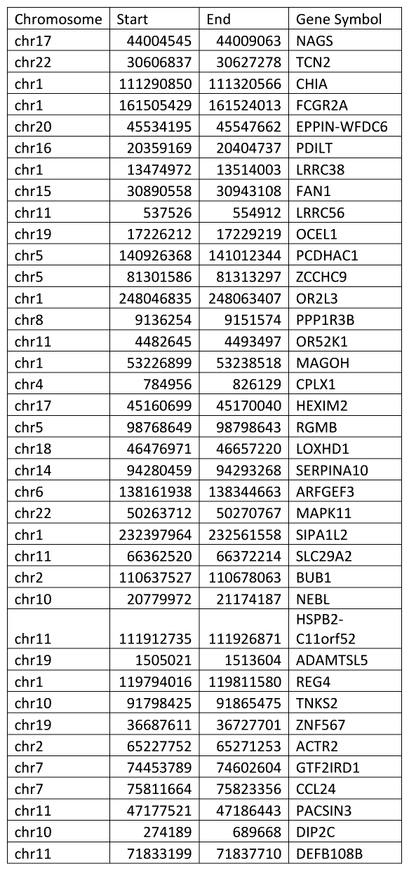

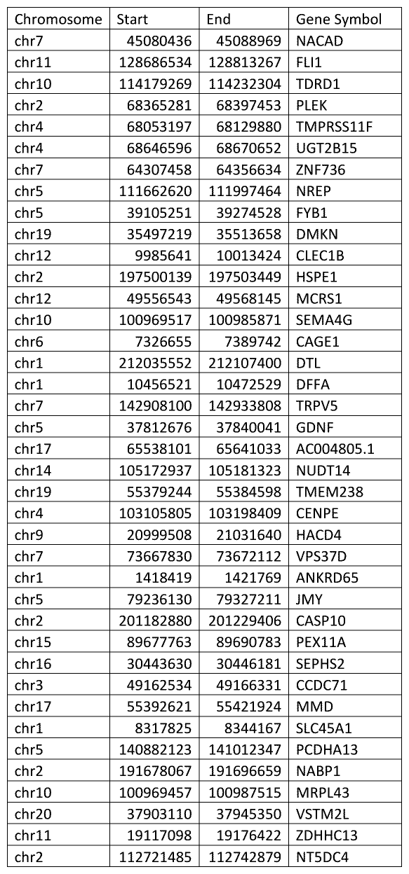

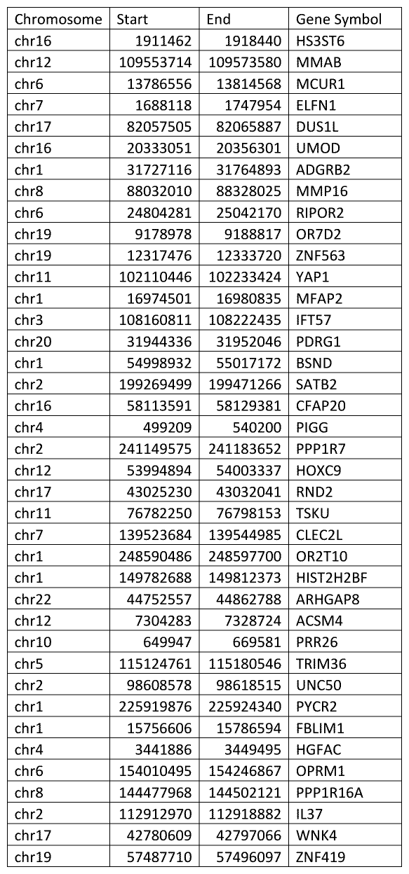

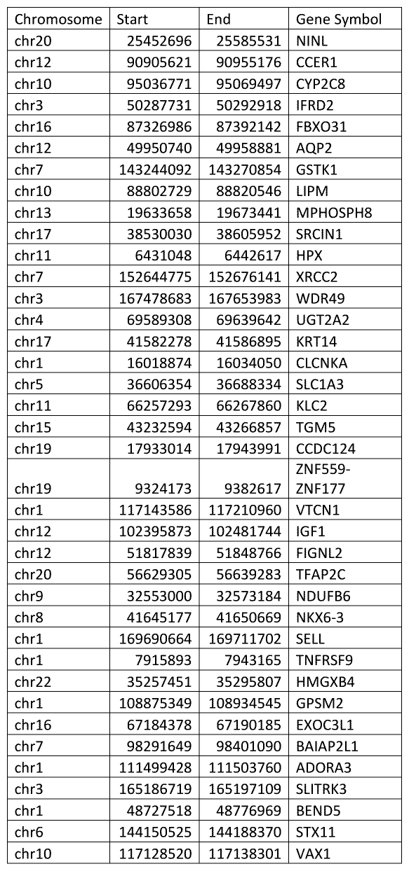

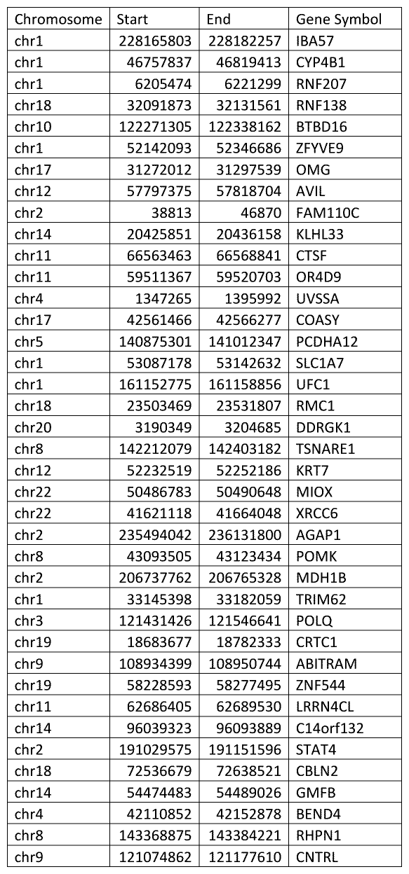

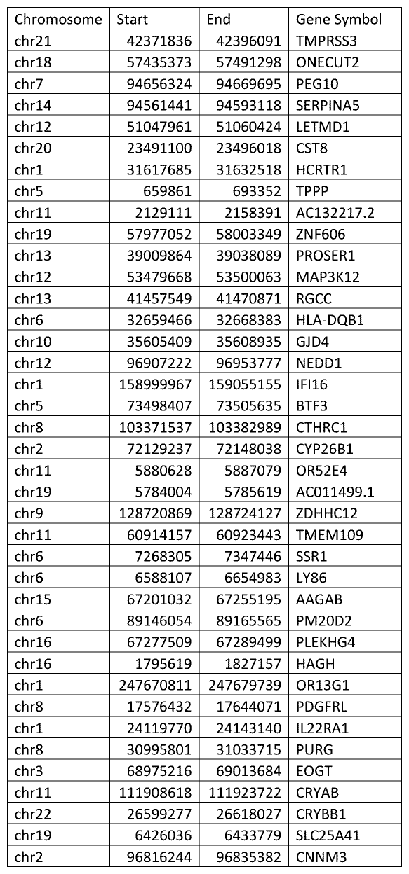

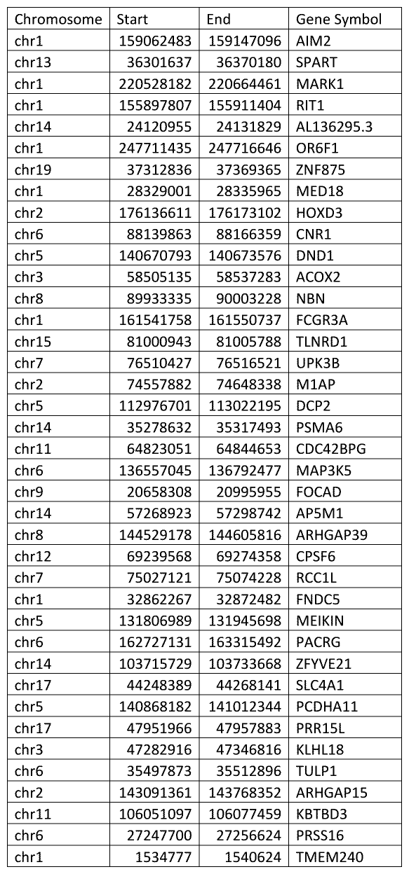

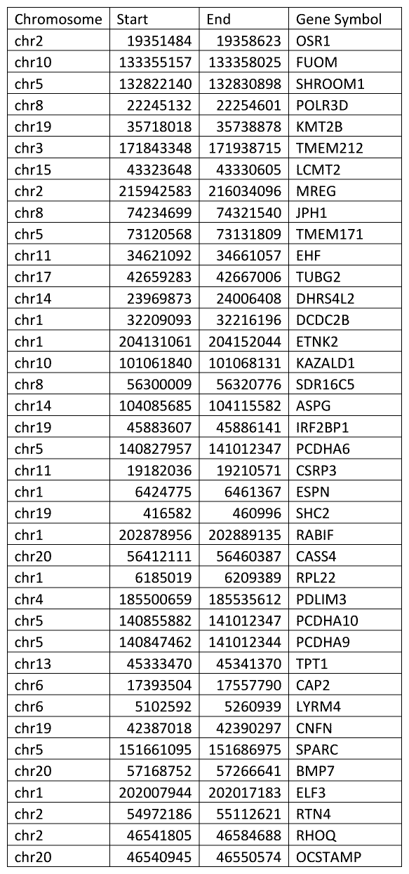

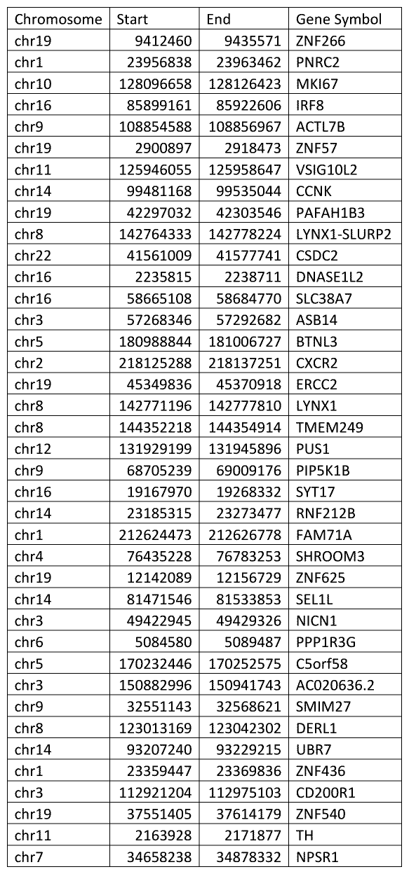

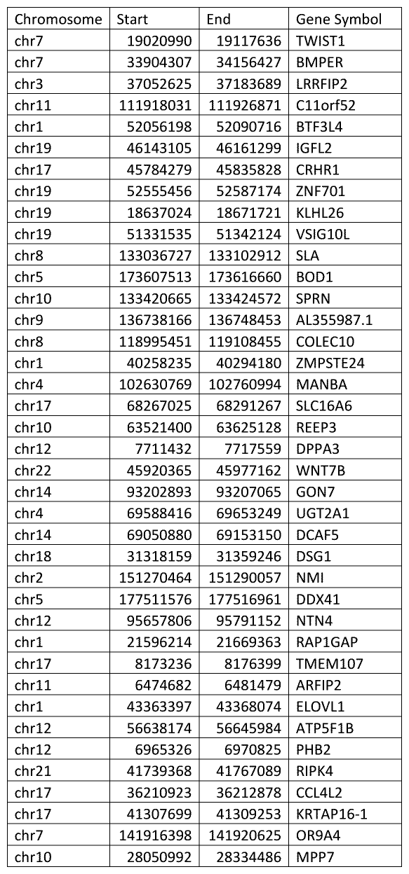

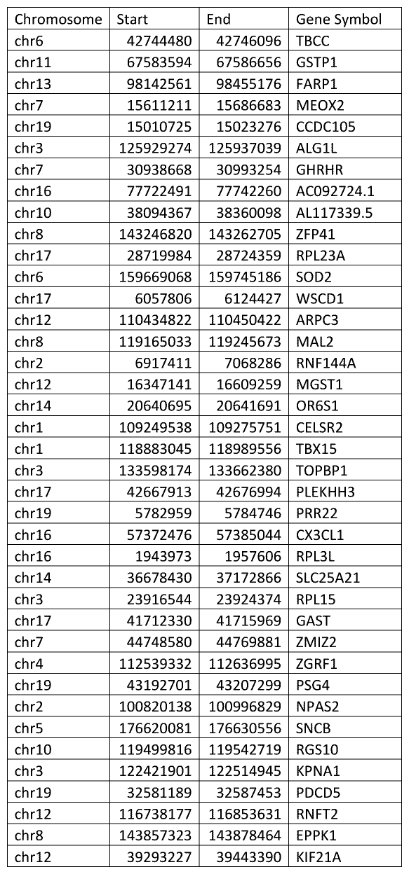

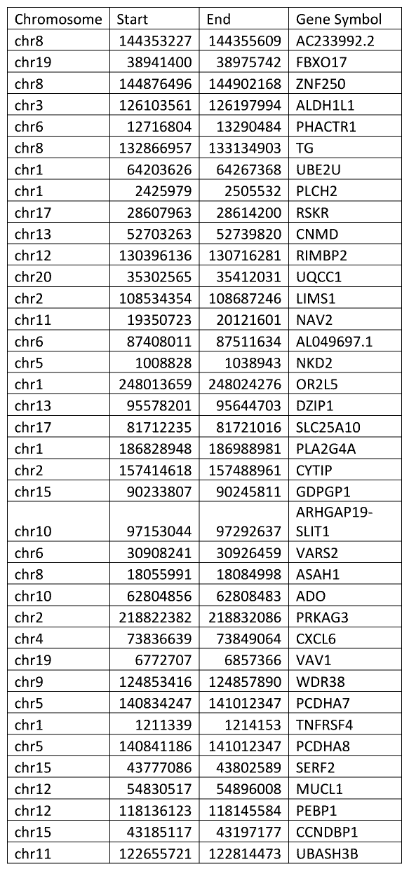

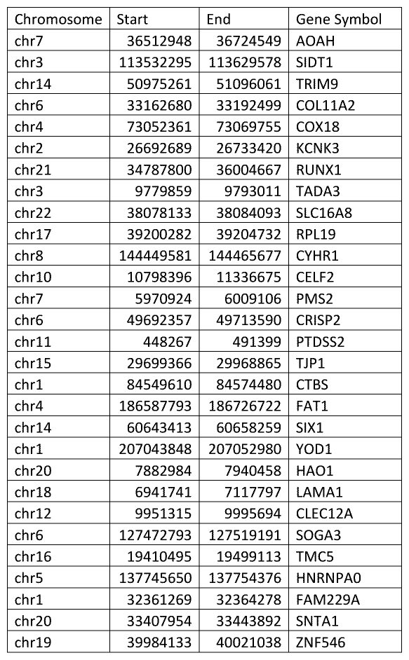

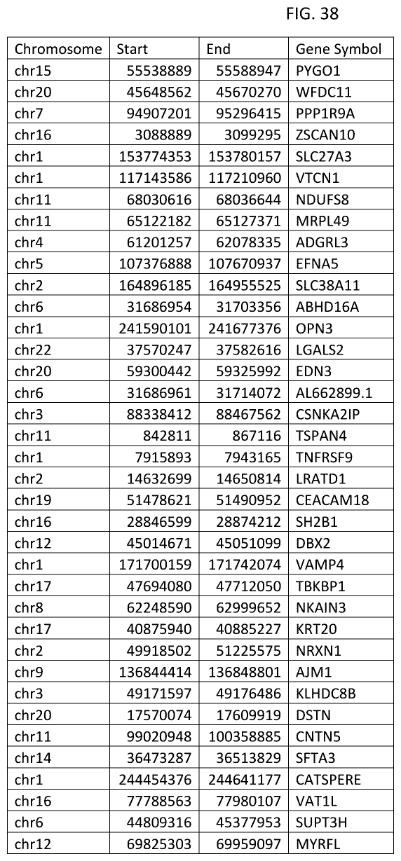

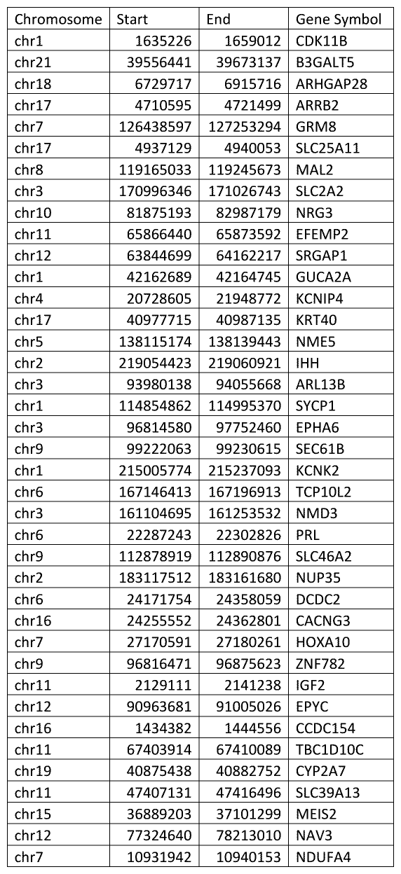

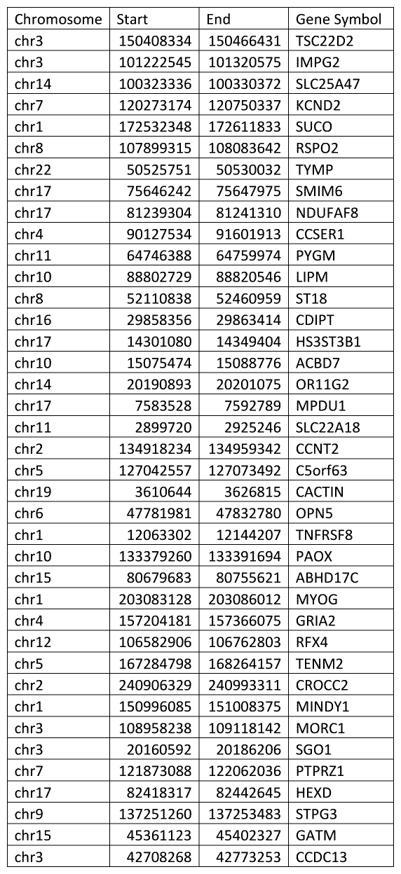

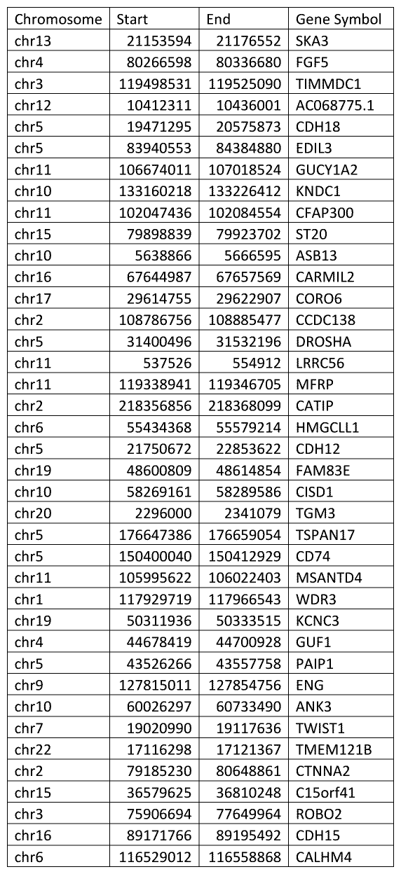

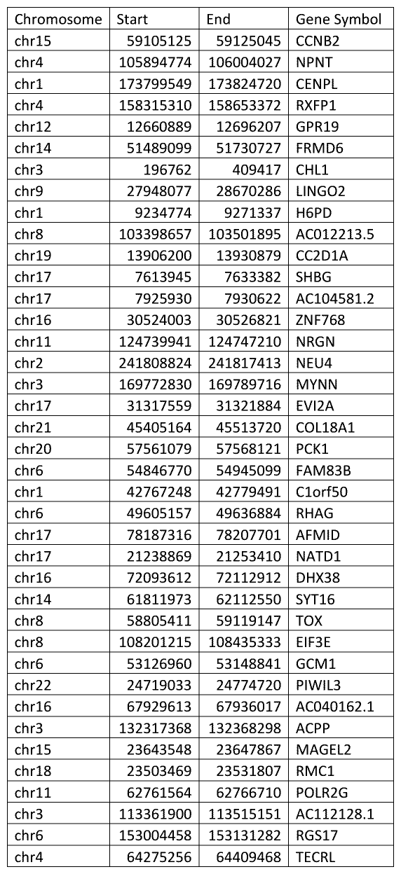

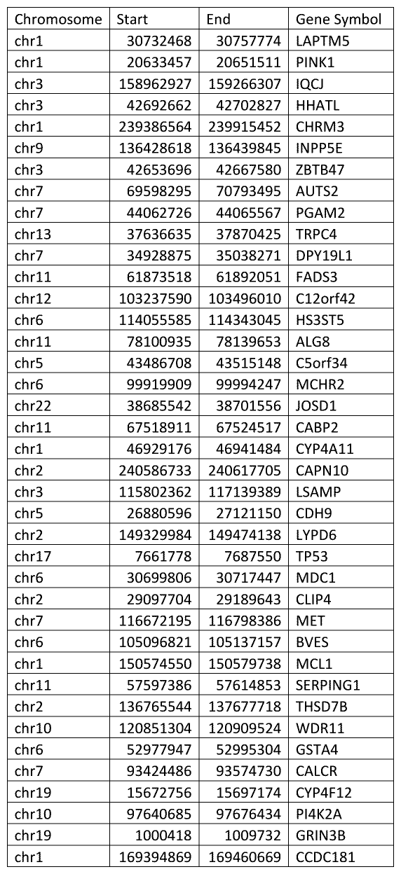

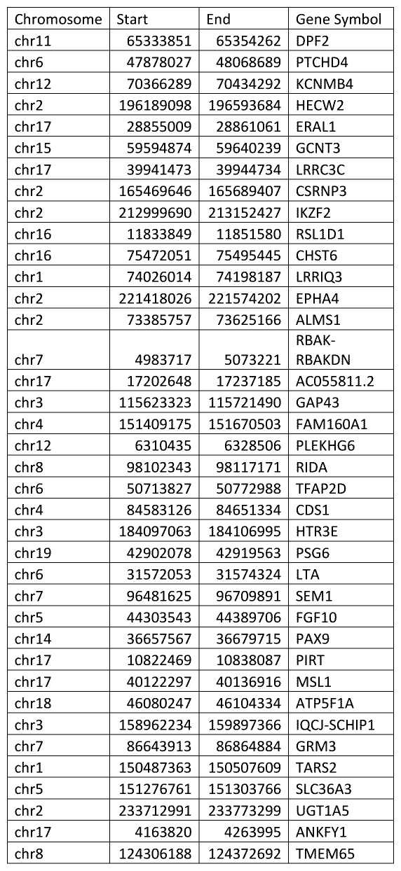

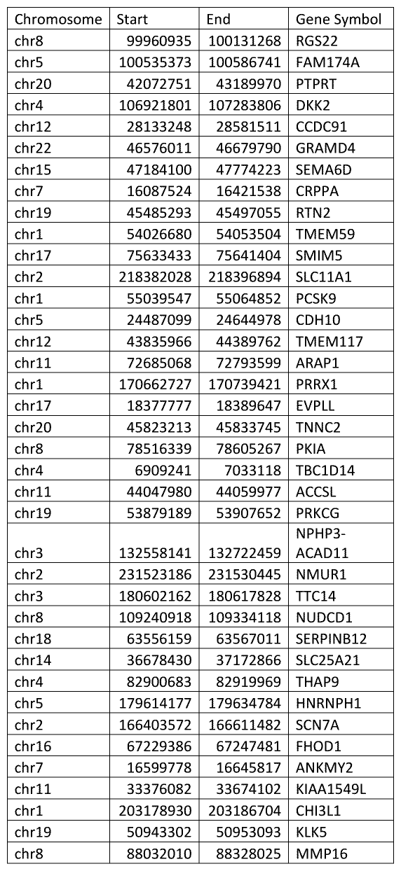

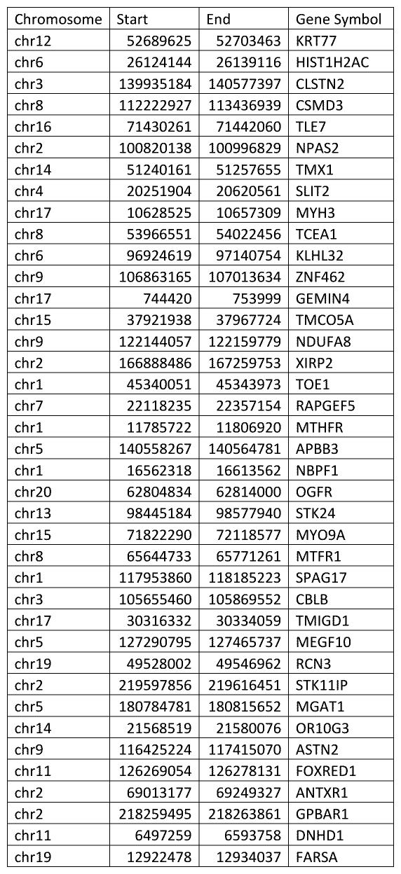

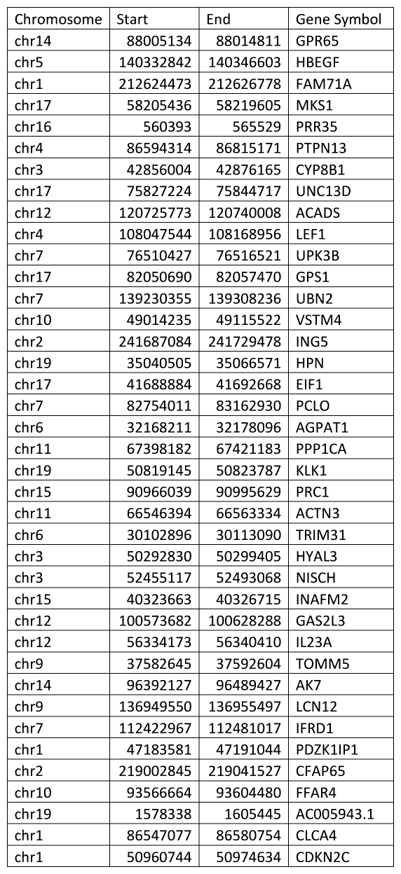

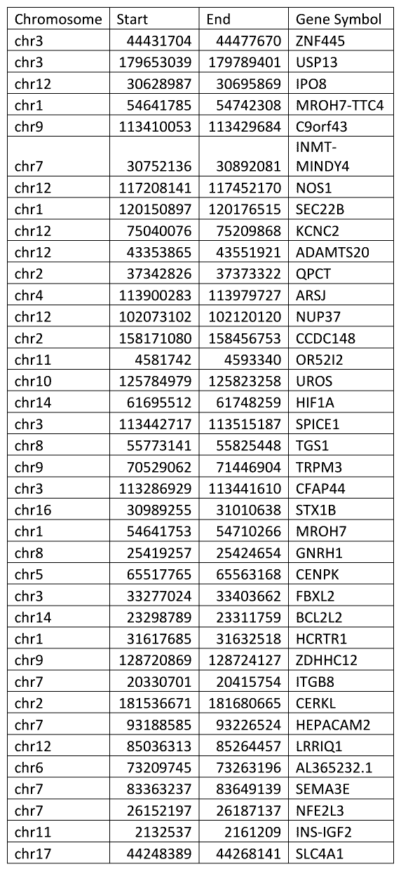

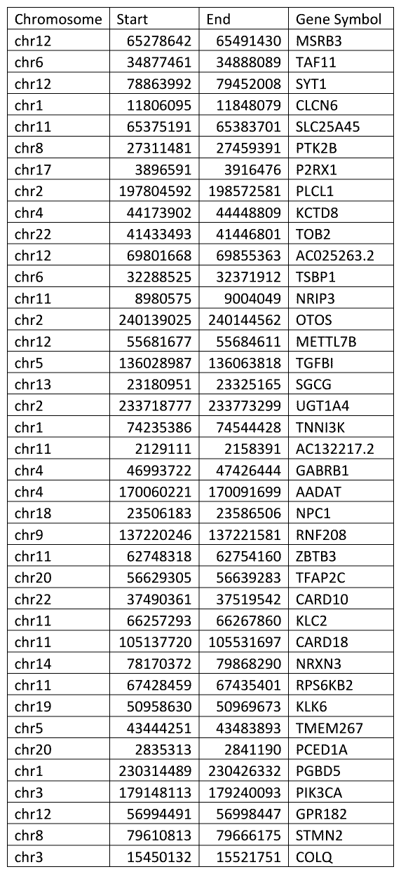

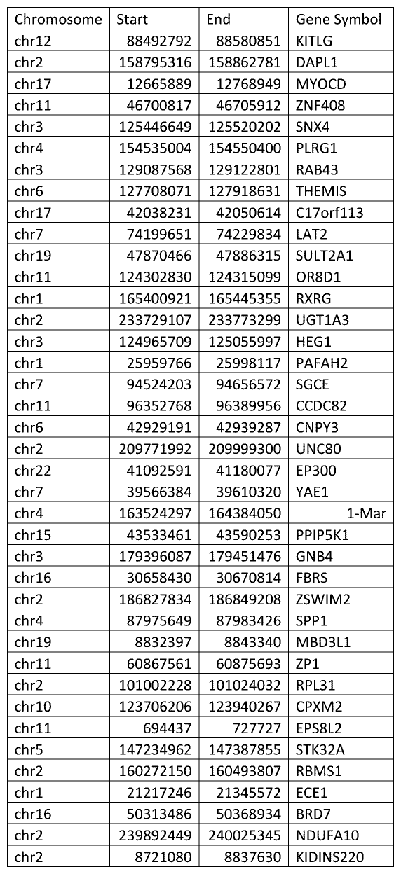

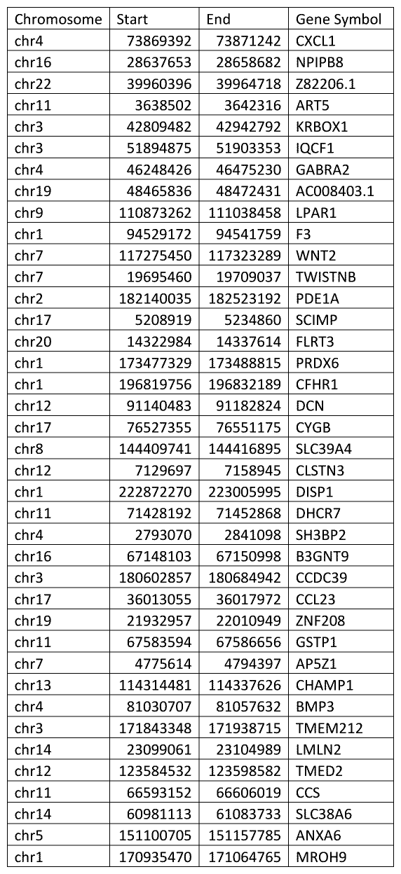

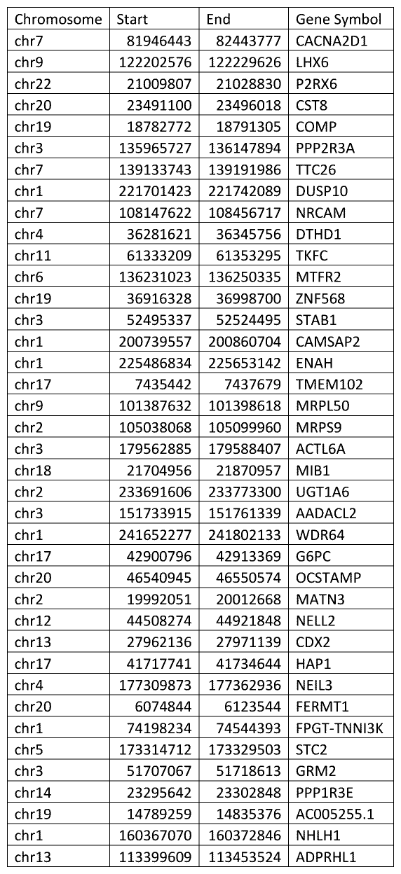

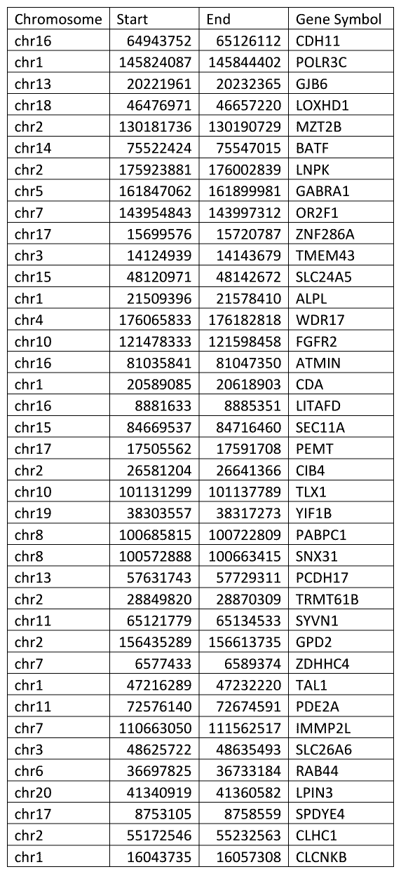

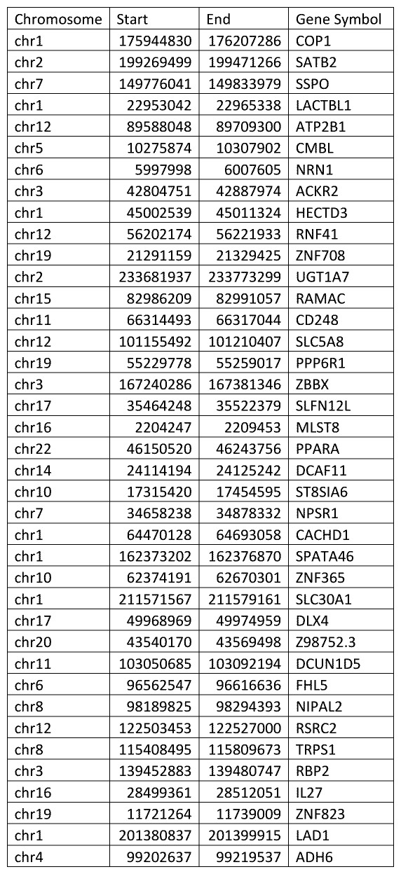

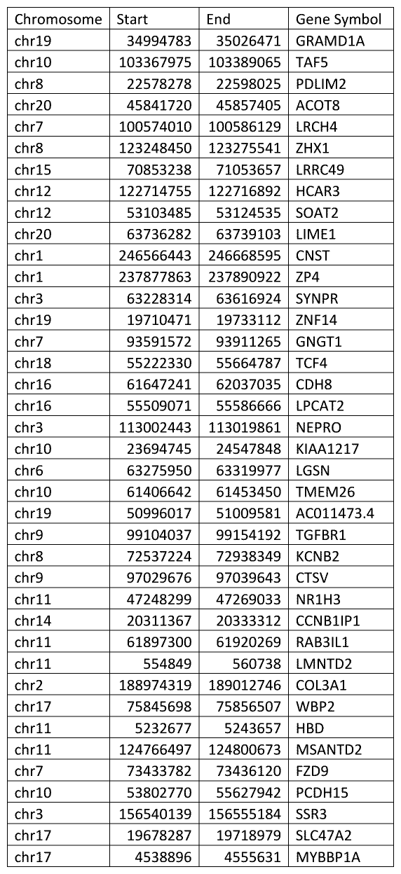

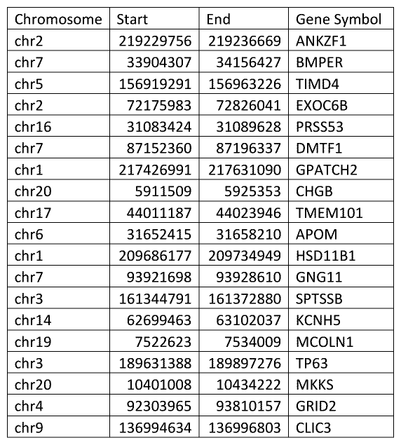

The selected loci in the presently described method are hydroxymethylation biomarkers, i.e., loci that have been identified herein as differentially hydroxymethylated in a manner that relates to the likelihood that a lung cancer patient will respond to treatment with an immunotherapy and/or to the likelihood that a lung cancer patient is responding to ongoing treatment with an immunotherapy. These biomarkers are set forth in FIGS. 29-32 AND FIGS. 36-38.

Both targeted and non-sequencing detection approaches after enrichment may also be used to quantitate specific hydroxymethylation biomarkers and loci of interest, if genome-wide coverage through shotgun sequencing is not required or desirable (generally for cost reasons). For example, after 5hmC enrichment, targeted PCR amplicons covering only specific regions may be generated from the 5hmC-enriched templates and employed as a narrower genome coverage approach, and used as input to sequencing or detected directly.

When a smaller number of discrete loci are of interest, the combination of these post-enrichment approaches with target amplification may also be an efficient way to reduce the number of sequencing reads (and sequencing costs) required for each sample, enabling further sample multiplexing per sequencing run and further reducing the sequencing costs required for each sample). In non-sequencing approaches, quantitative PCR or even hybridization assays could themselves be used as the quantitative readouts of the hydroxymethylation biomarkers (e.g., using direct fluorescence nucleotide labeling and microarray or other substrate capture and binding); such approaches are well known in the art, and frequently scaled to hundreds or even thousands of short amplicons.

In the present process, a 5hmC UFI sequence is added to the termini of the pulled down adapter-ligated dsDNA template molecules, so that the after amplification, pooling, and sequencing, information regarding hydroxymethylation profile can be deduced from the sequence reads obtained. That is, the sequence reads are analyzed to provide a quantitative determination of which sequences are hydroxymethylated in the cfDNA. This may be done by, e.g., counting sequence reads or, alternatively, counting the number of original starting molecules, prior to amplification, based on their fragmentation breakpoint and/or whether they contain the same molecular UFI. The use of molecular UFI sequences (or “molecular barcodes” as they are sometimes called) in conjunction with other features of the fragments (e.g., the end sequences of the fragments, which define the breakpoints) to distinguish between the fragments is known. See Casbon (2011) Nucl. Acids Res. 22 e81 and Fu et al. (2011) Proc. Natl. Acad. Sci. USA 108:9026-31), among others. Molecular barcodes are also described in U.S. Patent Publication Nos. 2015/0044687, 2015/0024950, and 2014/0227705, and in U.S. Pat. Nos. 8,835,358 and 7,537,897, as well as a variety of other publications.

Other methods of ascertaining the hydroxymethylation profile of DNA in the cell-free nucleic sample are described in International Patent Publication WO 2019/160994 A1 to Arensdorf et al. for “Methods for the Epigenetic Analysis of DNA, particularly Cell-Free DNA” and in U.S. Patent Publication No. 2017/0298422 to Song et al., previously incorporated by reference herein. These references are also useful in conjunction with an embodiment of the invention in which the present 5-hydroxymethylation determination and analysis further includes the detection of a cfDNA methylation profile in addition to the cfDNA hydroxymethylation profile.

The Arensdorf et al. methodology described in WO 2019/160994 can be implemented as follows:

Dual-Biotin Technique: After a cell-free nucleic acid sample has been extracted from a biological sample, with cfDNA having been adapter-ligated, 5hmC residues in the cfDNA are selectively labeled with an affinity tag, e.g., a biotin moiety as explained earlier herein. Biotinylation can be carried out by selective functionalization of 5hmC residues via βGT-catalyzed glucosylation with uridine diphosphoglucose-6-azide followed by a click chemistry reaction to covalently attach an alkyne-functionalized biotin moiety as explained previously. An avidin or streptavidin surface (e.g., in the form of streptavidin beads) is then used to pull out all of the dsDNA template molecules biotinylated at the 5hmC locations, which are then placed in a separate container for UFI sequence attachment during amplification. The remaining dsDNA template molecules in the supernatant are fragments that either have 5mC residues or have no modifications (the latter group including cDNA generated from cfRNA). A TET protein is then used to oxidize 5mC residues in the supernatant to 5hmC; in this case, a TET mutant protein is employed to ensure that oxidation of 5mC does not proceed beyond hydroxylation. Suitable TET mutant proteins for this purpose are described in Liu et al. (2017) Nature Chem. Bio. 13:181-191, incorporated by reference herein. The BGT-catalyzed glucosylation followed by biotin functionalization is then repeated. The fragments so marked-biotinylated at each of the original 5mC locations—are pulled down with streptavidin beads. The bead-bound DNA fragments are then barcoded—with a UFI sequence than used in the first step, i.e., a 5mC UFI sequence—during amplification. Unmodified DNA fragments, i.e., fragments containing no modified cytosine residues, now remain in the supernatant. If desired, sequence-specific probes can be used to hybridize to unmethylated DNA strands. The hybridized complexes that result can be pulled out and tagged with a further UFI sequence during amplification, as before.

Pyridine Borane Methodology: This is an alternative to the dual biotin technique, and is also a bisulfite-free process. The method relies on the use of pyridine borane, or an alternative, equally effective organic borane, to convert 5-carboxylcytosine (5caC) and 5-formylcytosine (5fC)—both of which can be generated from 5mC and 5hmC—to dihydrouracil (DHU). As DHU residues are read as thymine (T), while 5mC and 5hmC are read as C, the difference between parallel sequence reads enables the determination of DHU locations, which in turn indicates the location of 5mC and 5hmC locations.

In one embodiment, the pyridine borane method enables the identification of 5hmC locations in adapter-ligated target DNA in a cell-free sample. Initially, target DNA is oxidized with an oxidizing reagent that converts 5hmC to 5caC or 5fC, where the oxidizing reagent selected does not affect 5mC. Oxidation may be carried out enzymatically, although chemical oxidizing reagents are preferred in this embodiment. Examples of suitable chemical oxidizing agents for use in carrying out the aforementioned conversion include, without limitation: a perruthenate anion in the form of an inorganic or organic perruthenate salt, including metal perruthenates such as potassium perruthenate (KRuO4), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO (2,2,6,6-tetramethyl-1-piperidinyloxy) combination. The modified DNA containing 5caC or 5fC in lieu of 5hmC is then treated with an organic borane effective to reduce, deaminate, and either decarboxylate or deformylate the oxidized 5hmC and provide DHU in place thereof. The DHU-containing DNA is amplified and sequenced to provide 5hmC-indicative sequence reads, insofar as the sequence reads can be readily compared to standard sequence reads obtained for the target DNA, where the change from C in the standard sequence reads to a T in the 5hmC-indicative sequence reads indicates a 5hmC location.

In another embodiment, the pyridine borane methodology is used to identify 5mC locations in adapter-ligated target DNA in a cell-free sample. In this case, 5hmC residues in the target DNA are, at the outset, tagged with an affinity tag that enables removal of 5hmC-containing fragments from the sample. For instance, 5hmC residues may be selectively glucosylated with uridine diphospho (UDP) glucose functionalized at the 6-position with an azide moiety, a step that is followed by a spontaneous 1,3-cycloaddition reaction with alkyne-functionalized biotin via a “click chemistry” reaction. The resulting biotinylated DNA target molecules can then be separated from the sample with a solid support functionalized with a biotin-binding protein (e.g., avidin or streptavidin). Remaining DNA in the sample will contain 5mC, but not 5hmC. In the next step, 5mC residues in the remaining DNA are enzymatically oxidized to 5caC or 5fC, followed by treatment with pyridine borane to convert the oxidized 5mC residues to DHU. A preferred enzyme useful as the oxidizing agent is a Ten-Eleven Translocation Enzyme (TET) family enzyme or a “TET catalytically active fragment” as defined in U.S. Pat. No. 9,115,386, the disclosure of which is incorporated by reference herein. A preferred TET enzyme in this context is TET2; see Ito et al. (2011) Science 333 (6047): 1300-1303. Following amplification and sequencing, a comparison of the sequence reads obtained with the standard sequence reads for the target DNA indicates the location of 5mC residues in the sample DNA, as the change from C to T (resulting from DHU substitution for 5mC) indicates a 5mC location.

In a further embodiment, the pyridine borane technique can be implemented to detect the locations of both 5mC and 5hmC residues in a single cell-free DNA sample. The method involves, for a first fraction of a cell-free DNA sample comprising adapter-ligated target DNA,
(a) blocking 5hmC residues with a blocking reagent to yield blocked 5hmC residues;
(b) enzymatically oxidizing 5mC residues to provide oxidized 5mC residues selected from 5caC, 5fC, and combinations thereof;
(c) converting the oxidized 5mC residues to DHU by treatment with pyridine borane, thereby providing first fraction DNA comprising blocked 5hmC residues and DHU at 5mC locations; and
(d) amplifying and sequencing the first fraction DNA to provide first fraction sequence reads in which the blocked 5hmC residues read as C and DHU reads as T.

Glucosylation is effective as a blocking technique, in which case the blocking reagent may be ß-glucosyltransferase and the resulting blocking group on the 5hmC residues is glucose.

For a second fraction of the same sample, the method further involves:
(e) oxidizing 5hmC residues with an oxidizing reagent effective to convert 5hmC residues to oxidized 5hmC residues without modifying 5mC residues, wherein the oxidized 5hmC residues are selected from 5caC, 5fC, and combinations thereof; and
(f) converting the oxidized 5hmC residues to DHU by treatment with pyridine borane, thereby providing second fraction DNA comprising unmodified 5mC residues and DHU at 5hmC locations;
(g) amplifying and sequencing the second fraction DNA to provide second fraction sequence reads in which the unmodified 5mC residues read as C and DHU reads as T; and
(h) comparing the first fraction sequence reads with the second fraction sequence reads to identify 5mC and 5hmC locations in the template DNA.

See, e.g., Liu et al. (2019) Nature Biotech. 37:424-429.

The organic borane may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine. Further information concerning these organic boranes and reaction thereof to convert oxidized 5mC residues to DHU may be found in the Arensdorf patent publication cited above.

Biotin/Native 5mC Enrichment Method: This is an alternative to the dual biotin technique, and begins with biotinylation of 5hmC residues in adapter-ligated DNA fragments, followed by avidin or streptavidin pull-down. Here, however, instead of modifying the methylated DNA that remains in the supernatant, an anti-5mC antibody or an MBD protein is used to capture and pull down native 5mC-containing fragments. This technique is less preferred herein, insofar as it does not result in the generation of dsDNA template molecules that can be amplified, pooled, and sequenced with other dsDNA template molecules deriving from the same sample.

### 3. Hydroxymethylation Analysis:

The extent and location of the differences in patient cfDNA hydroxymethylation signature taken prior to undergoing therapy, i.e., the baseline hydroxymethylation signature, relative to a hydroxymethylation signature taken during therapy or to a reference hydroxymethylation profile, are then used to calculate a probability score representing the likelihood that the lung cancer patient will respond to a particular lung cancer therapy or that a lung cancer patient undergoing a lung cancer therapy is benefiting from the treatment. This may also be carried out using a 5hmC molecular response score approach as described earlier herein and discussed in the Example.

In a related embodiment, a method is provided for monitoring a lung cancer patient during lung cancer therapy, which involves, at the outset, obtaining hydroxymethylation monitoring data for the lung cancer patient by: (i) obtaining a cell-free DNA (cfDNA) sample from the patient, enriching for hydroxymethylated DNA in the sample, amplifying the hydroxymethylated DNA, and sequencing the amplified hydroxymethylated DNA in a manner that identifies 5-hydroxymethylcytosine (5hmC)-containing fragments or sites in the DNA; and (ii) measuring hydroxymethylation levels in the sequenced cfDNA at each of a plurality of hydroxymethylation biomarker loci, wherein each hydroxymethylation biomarker locus exhibits an increase or decrease in hydroxymethylation in a manner that correlates with a likelihood that the patient is responding to the lung cancer therapy. Then, the measured hydroxymethylation levels are input into a computer-generated predictive model that comprises a trained machine learning model; finally, the predictive model is used to generate the probability score, i.e., a score representing the likelihood that the patient is responding to the lung cancer therapy. As before, the 5hmC molecular response score approach may also be used, in addition to or as an alternative to the foregoing method.

More specifically, in order to calculate the probability score or 5hmC-based molecular response score, the methods of the invention include statistical analyses and mathematical modeling used to analyze high-dimensional and multimodal biomedical data, i.e., the data obtained using the present methods for comparing hydroxymethylation profiles. The methods make use of one or more objective algorithms, models, and analytical methods that include mathematical analyses based on topographic, pattern-recognition based protocols, e.g., support vector machines (SVM), linear discriminant analysis (LDA), naive Bayes (NB), and K-nearest neighbor (KNN) protocols, as well as other supervised learning algorithms and models, such as Decision Tree, Perceptron, and regularized discriminant analysis (RDA), and similar models and algorithms well-known in the art (Gallant S I, “Perceptron-based learning algorithms,” Perceptron-based learning algorithms 1990; 1 (2): 179-91).

Statistical analyses include determining mean (M), e.g., geometric mean, standard deviations (SD), Geometric Fold Change (FC), and the like. Whether differences in hydroxymethylation levels are deemed significant may be determined by well-known statistical approaches, typically by designating a threshold for a particular statistical parameter, such as a threshold p-value (e.g., p<0.05), a threshold S-value (e.g., ±0.4, with S≤−0.4 or S>0.4), or other value, at which differences are deemed significant, for example when the level of biomarker hydroxymethylation in a hydroxymethylation profile is considered significantly increased or decreased, respectively, relative to the hydroxymethylation level at the same hydroxymethylation biomarker locus in a reference hydroxymethylation profile.

In one aspect, the methods of the invention apply the mathematical formulations, algorithms or models to distinguish between normal and cancerous samples, and between various sub-types, stages, and other aspects of disease or disease outcome. In another aspect, the methods are used for prediction, classification, prognosis, and treatment monitoring and design.

For the comparison of hydroxymethylation levels or other values, data are compressed. Compression typically is by Principal Component Analysis (PCA) or a similar technique for visualizing the structure of high-dimensional data. PCA is used to reduce dimensionality of the data (e.g., measured expression values) into uncorrelated principal components (PCs) that explain or represent a majority of the variance in the data, such as about 50, 60, 70, 75, 80, 85, 90, 95 or 99% of the variance. PCA allows the visualization of biomarker levels and the comparison of hydroxymethylation profiles, such as between normal or reference samples and test samples. PCA mapping, e.g., 3-component PCA mapping is used to map data to a three-dimensional space for visualization, such as by assigning first, second, and third PCs to the x-, y-, and z-axes, respectively.

In some embodiments, there is a linear correlation between hydroxymethylation levels of two or more biomarkers. Pearson's Correlation (PC) coefficients may be used to assess linear relationships (correlations) between pairs of values, such as between hydroxymethylation levels of a biomarker. This analysis may be used to linearly separate distribution in expression patterns, by calculating PC coefficients for individual pairs of the biomarkers (plotted on x- and y-axes of individual Similarity Matrices). Thresholds may be set for varying degrees of linear correlation, such as a threshold for highly linear correlation of (R.sup.2>0.50, or 0.40). Linear classifiers can be applied to the datasets. In one example, the correlation coefficient is 1.0.

In some embodiments, Feature Selection (FS) is applied to remove the most redundant features from a dataset, such as a hydroxymethylation biomarker dataset. FS enhances the generalization capability, accelerates the learning process, and improves model interpretability. In one aspect, FS is employed using a “greedy forward” selection approach, selecting the most relevant subset of features for the robust learning models. (Peng H, Long F, Ding C, “Feature selection based on mutual information: criteria of max-dependency, max-relevance, and min-redundancy,” IEEE Transactions on Pattern Analysis and Machine Intelligence, 2005; 27 (8): 1226-38). In some embodiments, SVM algorithms are used for classification of data by increasing the margin between the n data sets (Cristianini N, Shawe-Taylor J. An Introduction to Support Vector Machines and other kernel-based learning methods. Cambridge: Cambridge University Press, 2000).

Analytic classification of the hydroxymethylation biomarkers herein can be made according to predictive modeling methods that set a threshold for determining the probability that a sample (e.g., a cfDNA sample obtained from a patient) belongs to a given class (e.g., increased likelihood that a lung cancer patient will respond to immunotherapy). The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, at least about 0.95, at least about 0.98, at least about 0.99, or higher.

Raw data can be initially analyzed by measuring the hydroxymethylation level for each biomarker. The data can be manipulated, for example, raw data can be transformed using standard curves, and the average of multiple measurements, if made, can be used to calculate the average and standard deviation for each patient. The data are then input into a selected predictive model, which will classify the sample. The resulting information can be communicated to a patient or health care provider, usually in the form of a written report.

In one embodiment, hierarchical clustering is performed in the derivation of a predictive model, where the Pearson correlation is employed as the clustering metric. One approach is to consider a dataset as a “learning sample” in a problem of “supervised learning.” CART is a standard in applications to medicine (Singer, Recursive Partitioning in the Health Sciences (Springer, 1999)) and can be modified by transforming any qualitative features to quantitative features, sorting them by attained significance levels, and a selected regularization method then applied (e.g., elastic net or lasso).

In some embodiments, the predictive models include Decision Tree, which maps observations about an item to a conclusion about its target value (Zhang et al., “Recursive Partitioning in the Health Sciences,” in Statistics for Biology and Health (Springer, 1999.). The leaves of the tree represent classifications and branches represent conjunctions of features that devolve into the individual classifications.

The predictive models and algorithms may further include Perceptron, a linear classifier that forms a feed forward neural network and maps an input variable to a binary classifier (Gallant (1990), “Perceptron-based learning algorithms,” in IEEE Transactions on Neural Networks 1 (2): 179-191). In this model, the learning rate is a constant that regulates the speed of learning. A lower learning rate improves the classification model, while increasing the time to process the variable (Markey et al. (2002) Comput Biol Med 32 (2): 99-109).

As explained earlier herein, the invention provides a method for determining the likelihood that an individual with lung cancer is likely to respond to immunotherapy and a method for determining the likelihood that a lung cancer patient undergoing immunotherapy is benefiting from the treatment. The invention thus encompasses diagnostic, prognostic, and predictive uses of hydroxymethylation profiles, as well as uses in patient monitoring, evaluation of treatment options, and evaluation of treatment efficacy, wherein, in each method of use, the hydroxymethylation profile generated is optionally combined with additional biomarker information and/or clinical parameters. All of the methods involve the generation of a hydroxymethylation profile comprising measurements of hydroxymethylation levels at each of a plurality of hydroxymethylation biomarker loci, where the loci are selected so as to exhibit differential hydroxymethylation in lung cancer patients who are responders or nonresponders to treatment with an immunotherapy.

Among the provided predictive and diagnostic/monitoring methods are those which employ statistical analysis and biomathematical algorithms and predictive models to analyze the detected hydroxymethylation information. Some embodiments include methods and systems for analyzing the hydroxymethylation information in classification, staging, prognosis, treatment design, evaluation of treatment options, prediction of outcomes (e.g., predicting development of metastases), and the like.

Also provided are methods that use evaluation of hydroxymethylation levels at the biomarker loci in treatment response prediction and patient monitoring, including evaluation of a patient's response to treatment and patient-specific or individualized treatment strategies. In some embodiments, the methods are used in conjunction with treatment, for example, by generating a hydroxymethylation profile weekly or monthly before, after, or at the time of treatment. As the hydroxymethylation levels at certain biomarker loci correlate with the progression of disease, ineffectiveness or effectiveness of treatment, and/or the recurrence or lack thereof of disease, the regular generation of hydroxymethylation profiles within an extended monitoring or treatment period is useful. In some aspects, the information obtained may indicate that a different treatment strategy is preferable. Thus, provided herein are therapeutic methods, in which biomarker evaluation is performed prior to treatment, and then used to monitor therapeutic effects.

More specifically, at various points in time after initiating or resuming treatment, significant changes in hydroxymethylation levels at one or more of the biomarker loci may be seen, indicating that a therapeutic strategy, e.g., immunotherapy, is or is not successful, that a patient is likely to benefit from immunotherapy, or that a change in therapeutic approach is advised. In some embodiments, the therapeutic strategy is changed following a hydroxymethylation analysis, such as by adding a different therapeutic intervention, either in addition to or in place of a prior approach, by increasing or decreasing the aggressiveness or frequency of the approach, or by stopping or reinstituting a treatment regimen.

### 4. Analysis of Multiple Feature Types:

The method of the invention may also involve a consideration of one or more additional feature types in combination with the 5-hydroxymethylation analyses described above. That is, the probability score representing the likelihood that a patient will respond to lung cancer therapy or is responding to lung cancer therapy takes into account not only the patient's 5-hydroxymethylation levels at specific 5hmC biomarker loci but also one or more additional feature types that correlate with the likelihood that the patient will respond to treatment with lung cancer therapy, particularly immunotherapy. The additional feature may be an additional type of biological marker. That is, the cell-free DNA sample obtained from the patient, in addition to being analyzed in terms of hydroxymethylation levels at various loci, may also be analyzed with respect to biomarkers such as methylation levels; DNA fragment size and fragment size distribution; cell-free DNA concentration in a patient sample, corresponding to cfDNA plasma concentration ([p-cfDNA]); RNA analysis such as T cell-inflamed gene expression profile (GEP) (see Cristescu et al. (2018), cited previously); changes in circulating tumor DNA (ctDNA) count; the surrogate markers for tumor neoantigens microsatellite instability-high (MSI-H), deficient mismatch repair (dMMR), and tumor mutational burden, which can be measured from tissue samples or plasma samples (see Thompson et al. (2021), “Serial Monitoring of Circulating Tumor DNA by Next Generation Gene Sequencing as a Biomarker of Response and Survival in Patients with Advanced NSCLC Receiving Pembrolizumab-Based Therapy,” JCO Precision Oncology 5:510-524) and Bindal et al. (2021), “Biomarkers of therapeutic response with immune checkpoint inhibitors,” Ann. Transl Med 9 (12): 1040); expression of the immune suppression biomarkers LAG3 and IDO-1; regulatory T-cell count (Tregs); myeloid derived suppressor cell count; inflammation gene signatures; tumor infiltrating effector cells; lymphocyte count; microbiome composition; germline mutations; and the like.

Patient-specific clinical parameters may also be considered in combination with the hydroxymethylation analysis. These covariates include factors such as lesion size; lesion grade; lesion stage; lesion location; patient age; patient weight; patient body mass index (BMI), patient gender; patient ethnicity; cigarette smoking history; and exposure or lack of exposure to a known carcinogen.

In one embodiment, an ensemble model, e.g., a stacked ensemble model, is used to combine multiple datasets and machine learning techniques to predict a lung cancer patient's likelihood of responding to treatment with a particular lung cancer therapy or to determine whether a lung cancer patient who is undergoing treatment is responding to the lung cancer therapy used. The model uses 5hmC count data within various annotated regions across the genome such as a gene body, promoter, 5′ UTR, 3′ UTR, enhancer, intron, exon, LINE, SINE, or the like. Each annotated region is considered a feature set and incorporated into the stacked ensemble. In addition to the 5hmC features, additional feature values are used in this embodiment that are determined from one or more additional feature types. For example, feature values can be determined from additional feature types such as cfDNA fragment size and size distribution, copy number variation, and cell-free DNA plasma concentration from a WGS library constructed for the cell-free DNA sample obtained from a patient.

In a representative embodiment, a WGS library derived from the patient cfDNA sample is GC-corrected and processed to determine within approximately 1 MB, 2 MB, 4 MB, 5 MB, or even 8 MB windows the number of fragments in two or more different size ranges, e.g., two, three, four, five, or more different size ranges within the fragment size distribution obtained. Examples include two size ranges of 100-150 bp and 150-220 bp; two size ranges of 100-150 bp and 150-300 bp; two size ranges of 100-150 bp and 150-400 bp; two size ranges of 120-155 bp and 155-200 bp; 50-150 bp and 150-400 bp; three size ranges of 100-160 bp, 160-200 bp, and 200-220 bp; three size ranges of 50-152 bp, 153-240 bp, and 241-1000 bp, and the like.

In another representative embodiment, instead of or in addition to a WGS library derived from the patient cfDNA sample, the number of fragments in two or more different fragment size ranges is taken from only those fragments having 5hmC sites, which can be isolated from the patient cfDNA sample as explained in Part 2 of this Section. That is, although the foregoing description pertains to fragment size evaluation in a WGS library, the 5hmC-containing fragments can be evaluated in the same way, and used in addition to or instead of the WGS fragment size analysis.

Although the ratio of the number of large fragments to the number of small fragments can be employed as a single feature, it is preferred that the absolute number of fragments in a particular size range be used as an individual feature, such that, for example, for two size ranges, the number of fragments in the first size range (e.g., 100-150 bp) serves as a first feature and the number of fragments in the second size range (e.g., 150-220 bp) serves as a second feature. As another example, for three size ranges, the number of fragments in the first size range (e.g., 100-160 bp), the number of fragments in the second size range (e.g., 160-200 bp), and the number of fragments in the third size range (e.g., 200-220 bp) serve as three distinct features.

The foregoing features, i.e., the number of fragments within each of two or more specific size ranges, may be combined with at least one other feature type, in addition to the patient hydroxymethylation profile, in the analysis that follows. Copy number variation (CNV) is one such additional feature type. This may be readily determined from the GC-corrected WGS library. For instance, the number of reads of length 50-1000 bp, or another selected length, can be mapped in individual windows, e.g., 100 kb windows, along the genome to support detection of CNV. Cell-free DNA concentration in the patient sample can serve as yet an additional feature to be combined with hydroxymethylation profile and at least one of CNV and number of fragments in different size bins. Concentration of cfDNA in the patient sample can be readily determined by methods described in the pertinent literature or known to those of ordinary skill in the art. See, e.g., Chen et al. (2021) Nature Portfolio 11:5040, incorporated herein by reference.

After normalizing by total counts for each feature type, i.e., number of fragments in each of two or more size ranges elastic net regression models are built using glmnet. The elastic net mixing ratio a can be optimized, for instance, using k-fold (e.g., 5-fold, 10-fold, greater than 10-fold) cross validation (e.g., set to 0.01, 0.1, 0.5, or the like) for each feature set. The regularization parameter λ is optimized at run time per feature set, again using k-fold (e.g., 5-fold, 10-fold, or greater than 10-fold) cross validation.

The models built for all feature types—e.g., number of fragments in each of two or more size ranges, CNV, plasma cfDNA concentration, and 5hmC profiles—are combined together using a final elastic net fit with a predetermined elastic net mixing ratio (e.g., α=0.01,0.1, 0.5, or the like) in a stacked ensemble fashion. The stacked ensemble combines the models by using the individual predictive scores from each separate model as a feature vector, then fitting for coefficients that weight the scores from each model. By way of illustration rather than limitation, the non-zero coefficients from the individual models can roughly comprise: 60-90% hydroxymethylation profile and 10-40% number of fragments within at least two size ranges. When CNV and cfDNA concentration are included, the relative weighting may be, as an example, 60-90% hydroxymethylation profile, 1-20% number of fragments within at least two size ranges, 1-20% CNV, and 1-20% cfDNA concentration. In one specific example, with cfDNA concentration omitted, the relative weighting may be 75-85% hydroxymethylation profile, 14-24% CNV, and 1% fragmentation.

A new sample can then be scored as follows. First, the 5hmC and WGS libraries are processed to prepare the feature vectors used by the individual elastic net models which are input into the full stacked ensemble model. The individual elastic net model predictive scores are computed from the appropriate (5hmC or WGS) feature vector. Then, those scores are passed into the full stacked ensemble model as input to generate a final probability score.

### Example

### A. Study Design and Methods:

### (i) Clinical Cohorts and Study Design:

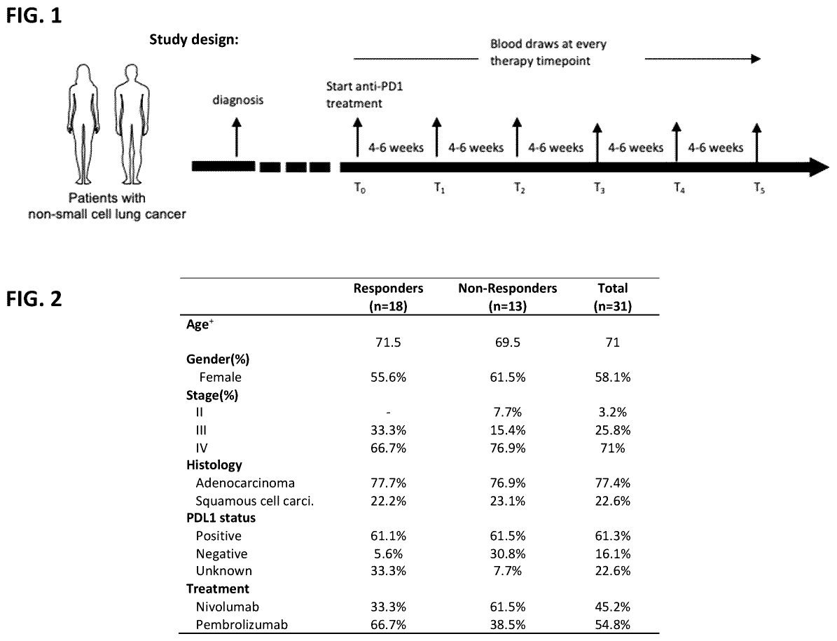

This study was performed using plasma obtained from subjects with non-small cell lung cancer who underwent pembrolizumab or nivolumab monotherapy. 31 patients were enrolled at participating sites in Germany with written informed consent for use of blood specimens for archival storage and retrospective analyses. A total of 151 blood samples were collected as approved by the Institutional Review Boards (IRBs) responsible at each site. The study protocol submission, IRB approval and specimen handling across all sites were managed by Indivumed GmbH (Hamburg, Germany). To identify changes that are induced in the 5-hydroxymethylome of plasma-derived cfDNA, a cohort of patients with non-small-cell lung cancer (NSCLC) was assembled who received anti-PD-1 immunotherapy agents pembrolizumab or nivolumab as monotherapy (FIG. 1). The median age for the study cohort was 71. Female subjects made up 58.1% of the cohort. The majority of the patients were late stage (96.8%). Adenocarcinomas constituted 77.4% and squamous cell carcinoma 22.6% of the study cohort. Blood samples were collected at baseline and after commencement of treatment in 4-6 week intervals; the timeline for both anti-PD1 immunotherapy dosing and blood draws is shown in FIG. 1. The complete cohort consisted of 31 patients and 150 blood samples total. Response to therapy was measured by radiological imaging.

To identify the potential for 5hmC-based biomarkers to provide information on immunotherapy response in lung cancer patients, patient cfDNA was first isolated from plasma, then subjected to a 5hmC enrichment assay in which 5-hydroxymethylated cfDNA fragments were pulled down with a highly specific and sensitive chemical click reaction followed by DNA library preparation. Whole genome libraries were prepared from the same input cfDNA material. Genomic regions enriched for 5hmC were determined by peak detection using MACS2 (https://github.com/taoliu/MACS). Details of the procedures used are as follows:

(ii) Plasma collection: Whole blood specimens obtained by routine venous phlebotomy in Streck Cell-Free DNA BCT® tubes according to the manufacturer's protocol (Streck, La Vista, NE). The tubes were maintained at 15° C. to 25° C. until plasma isolation. Plasma was isolated within 24 hr of phlebotomy by centrifugation of whole blood at 1600 x g for 10 min at room temperature, followed by transfer of the plasma layer to a new tube for centrifugation at 1600×g for 10 min. Plasma was then aliquoted and stored at −80° C.

(iii) Cell-free DNA isolation: Cell-free DNA was isolated using the MagMAX® cell-free DNA isolation kit (Thermo Fisher Scientific, Waltham, MA) following the manufacturer's protocol with automated runs on HAMILTON STAR liquid handlers (HAMILTON Company, Reno, NV) using the MagMAX magnetic beads. During this procedure, plasma was incubated with Proteinase K and 20% SDS at 60° C. for 20 minutes followed by cooling. Next, cfDNA was bound to the magnetic beads and washed with a Thermo Fisher Scientific proprietary wash buffer and with 80% ethanol. Finally, cfDNA was eluted in 75 μl elution buffer. All cfDNA eluates were quantitated using Molecular Devices' Spectramax® Plate Readers using the PicoGreen® dsDNA quantitation assay (Thermo Fisher Scientific). TapeStation® 4200 capillary electrophoresis (Agilent Technologies, Santa Clara, CA) was employed to ensure the absence of contaminating high molecular weight DNA emanating from white blood cell lysis.

(iv) 5-Hydroxymethylcytosine (5hmC) enrichment assay and 5hmC/WGS library preparation: 5hmC-enriched libraries were prepared using the cell-free “5hmC-Seal” method described in International Patent Publication WO 2017/176630 to Quake et al., Song et al. (2011) 29:68-72, and Han et al. (2016) Mol. Cell 63:711-19, the disclosures of which are incorporated by reference herein. Briefly, hMe-Seal is a low-input, whole-genome cell-free 5hmC sequencing method based on selective chemical labeling, in which β-glucosyltransferase is used to selectively label 5hmC with a biotin moiety via an azide-modified glucose for pull-down of 5hmC-containing DNA fragments for sequencing. In implementing hMe-Seal in the present case, the cfDNA was normalized to 10 ng total input for each assay and ligated to sequencing adapters, followed by selective labeling of 5hmC with β-GT, and affinity enrichment via selective pull-down of DNA fragments containing biotin-labeled 5hmC by binding to Dynabeads M270 Streptavidin (Thermo Fisher Scientific). PCR was then carried out directly on the beads to minimize sample loss during purification. All libraries were quantitated by Molecular Devices's SpectraMax Plate Readers using the PicoGreen® dsDNA quantitation assay (Thermo Fisher Scientific) and normalized to 1 ng/μl prior to pooling. Library pools were quantitated by Qubit dsDNA High Sensitivity Assay (Thermo Fisher Scientific) and normalized in preparation for sequencing.

(v) DNA sequencing and alignment: DNA sequencing was performed according to manufacturer's recommendations with 75 base-pair, paired-end sequencing using a NovaSeq® instrument with version 2 reagent chemistry (Illumina, San Diego, CA). Data was collected using NovaSeq System Suite 2.2.04. Raw data processing and demultiplexing was performed using version 2.20.0.422 of the Illumina bcl2fastq software to generate sample-specific FASTQ output. Sequencing reads were aligned to the hg38 reference genome using BWA-MEM with default parameters (Li and Durbin (2010), “Fast and accurate long-read alignment with Burrows-Wheeler transform,” Bioinformatics 26:589-595). Sequencing data quality was assessed using the Picard tool kit (Broad Institute).

(vi) Peak detection: BWA-MEM read alignments were employed to identified regions or peaks of dense read accumulation that mark the location of a hydroxymethylated cytosine residue. Prior to identifying peaks BAM files containing the locations of aligned reads were filtered for poorly mapped (MAPQ<30) and not properly paired reads using SAMtools (Li et al. (2009), “The Sequence Alignment/Map format and SAMtools,” Bioinform Oxf Engl 25:2078-9). 5hmC peak calling was carried out using MACS2 (https://github.com/taoliu/MACS) with a p-value cut off of 1.00e-5. Identified 5hmC peaks residing in “blacklist regions” as defined elsewhere (https://sites.google.com/site/anshulkundaje/projects/blacklists) and residing on chromosomes X, Y and mitochondrial genome were also removed using Bedtools (Quinlan et al. (2010), “BEDTools: a flexible suite of utilities for comparing genomic features,” Bioinformatics 26:841-842). Computation of genomic feature enrichment overlapping 5hmC peaks was performed using the software HOMER (http://homer.ucsd.edu/homer/) with default parameters.

(vii) Differential 5-Hydroxymethylation Analysis:

For the purpose of identifying peaks with differential 5hmC representation, first, a bed file with a union of all identified peaks was generated and overlapping peaks were merged. Raw counts over the peaks were normalized by transforming raw counts to log 2 (counts per million). Weakly represented peaks (CPM>2 in fewer than 10 samples) were removed before differential analysis. To identify differential 5-hydroxymethylation induced by immunotherapy treatment, a Wilcoxon signed rank test was used to compare plasma collected at the baseline timepoint with the plasma collected while the patients were undergoing treatment. To identify differentially hydroxymethylated regions in responding patients relative to non-responding patients, the Wilcoxon sum test was applied to compare plasma hydroxymethylation profiles obtained at the baseline timepoint in responding versus non-responding patients. Peaks with a p-value of less than 0.05 and a log 2 fold change of at least 1.5 (either up or down) were retained for further analysis.

The 5hmC-based molecular response (MR_{5hmc}) was also calculated, starting with an evaluation of

log 2(T_{R}/T₀)
at each 5hmC biomarker locus, wherein T₀ is the baseline CPM at each locus and T_{Q} is the CPM seen during therapy monitoring at time Q at each locus. The values of log 2 (T_{Q}/T₀) obtained at each locus are designated as either xᵢ (a positive value when there is a response or a negative value when there is non-response) or yᵢ (a positive value when there is non-response or a negative value when there is a response). That is, the xᵢ and yᵢ represent genomic loci exhibiting increased 5hmC that is either positively or negatively correlated with treatment response, respectively. Then, MR_{5hmc} is calculated as the difference of the means:

$${MR}_{5 hmC} = {μ}_{x} - {μ}_{y}$$

where μₓ and μ_{y} are the means of all xᵢ, i=1, . . . , nᵢ, and yⱼ, j=1, . . . , nⱼ, respectively (where in this example, nⱼ=129 and nⱼ=154). In this analysis, the selected loci had a p-value of less than 0.05 and a difference & of at least 1.5, wherein

$$ζ = ( {T}_{Q} - {T}_{0} ) / {T}_{0} .$$

It should be noted that while T_{Q} indicates the CPM at each 5hmC biomarker locus at some time point Q during treatment, the term T_{R} is used to refer to the specific time point at which imaging is done and therapy response evaluated according to the RECIST guidelines (i.e., responder=CR/PR or non-responder=PD).

(viii) Predictive Modeling:

To assess the feasibility of detecting patients' response to therapy using the 5hmC profiling assay described above, cfDNA in plasma obtained from the cancer patients was profiled. The intention was to use the samples to provide a set suitable for training a machine learning model that would detect a signal of progression. An ensemble of binomial models (“base learners”) was trained on the early versus late stage samples, where the feature vectors for the various binomial models were based on different genomic features derived from our assay. For example, the feature vectors used for one of the binomial models consisted of cpm counts of fragments mapped to gene bodies. Each base learner binomial model was trained using elastic net regularization, a means of performing feature reduction when the number of features exceeds the number of samples. See Friedman et al. (2010) J. Stat. Software 33 (1): 1-22 for a description of the general elastic net procedure. Software implementation of these methods can be found at https://cran.r-project.org/web/packages/glmnet/index.html. Prior to fitting a base learner with elastic net, an initial filtering procedure was carried out which removed features with low variance. An elastic net logistic regression fit was performed using glmnet 40 with alpha, the mixing parameter, set to 0.01. The value of the elastic net regularization parameter lambda was set using cv.glmnet, which uses cross validation to pick an optimal value of lambda. After fitting the individual base learner binomials for each feature type vector, an elastic net binomial ensemble model was trained using the scores from the individual base learners setting alpha to 0.5 and again determining the value of lambda via cv.glmnet.

### B. Results:

### (i) Recist Evaluation:

As noted above, the complete cohort consisted of 31 patients, with 150 blood samples taken in total; blood samples were collected as described in A (i). Each patient's response to anti-PD1 immunotherapy was determined by radiological imaging. Of the 31 patients, 18 exhibited a radiological response of partial or complete response (PR/CR) pursuant to the “Response Evaluation Criteria in Solid Tumors” (RECIST 1.1) guidelines (Eisenhauer et al. (2009), “New response evaluation criteria in solid tumours: Revised RECIST guideline (version 1.1)” European J. Cancer 45:228-247), while 13 patients showed progressive disease (FIG. 2).

(ii) 5hmC Profiles of the cfDNA Samples in Study Cohort:

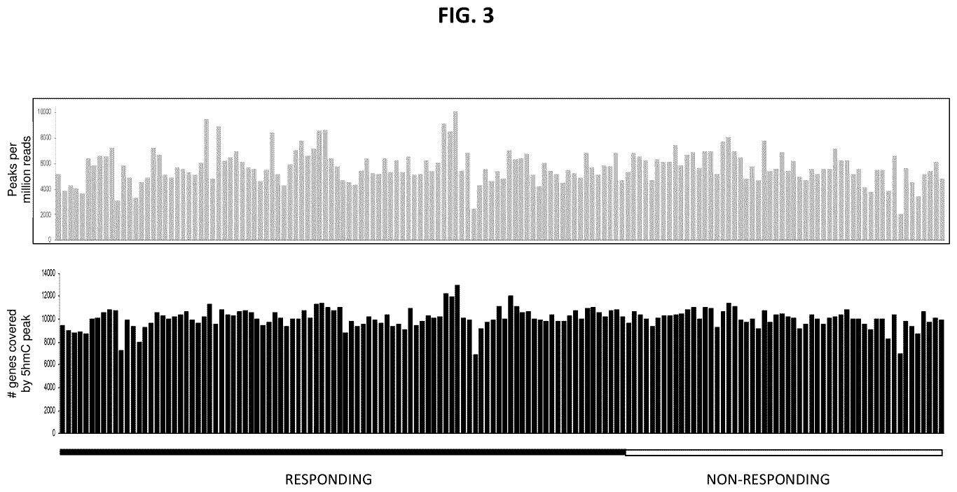

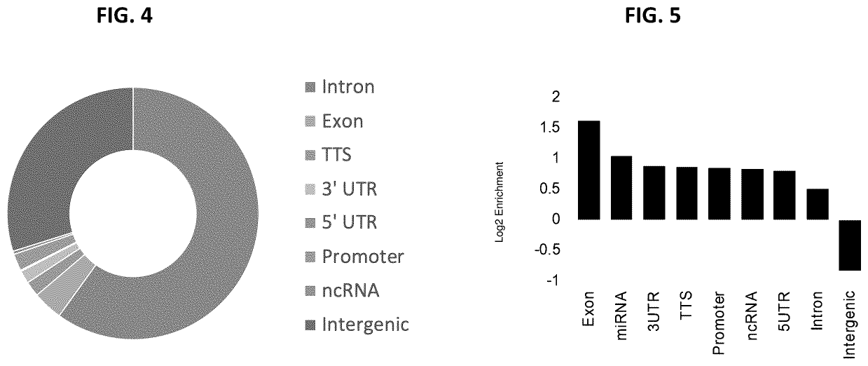

Following plasma collection, 5hmC enrichment, DNA sequencing and alignment, and the 5hmC enrichment assay described in Part A, sections (ii) through (v), peak detection was carried out using the methodology described in Part A, section (vi), with genomic regions enriched for 5hmC determined by peak detection using MACS2. The number of peaks discovered per million unique reads in a given sample ranged from 2,102 to 10,090 with a median of 5,635. See FIG. 3, which depicts the 5hmC landscape per sample as barplots showing the number of 5hmC peaks observed (top panel) and the number of genes/promoters that are hydroxymethylated (bottom panel). The majority of peaks localized to introns and intergenic regions, 60% and 30% respectively (FIG. 4). Consistent with previous publications (Garcia-Murillas et al. (2015), “Mutation tracking in circulating tumor DNA predicts relapse in early breast cancer.” Sci Transl Med 7: 302ra133; Guler et al. (2020) “Detection of early stage pancreatic cancer using 5-hydroxymethylcytosine signatures in circulating cell free DNA.” Nat Commun 11:5270), 5hmC peaks were most statistically enriched over genic regions as determined by the ratio of 5hmC bases in each genomic feature relative to the total number of bases in the same feature genome-wide (FIG. 5).

(iii) Differential 5-Hydroxymethylation of Plasma cfDNA in Anti-PD-1 Responding and Non-Responding Patients:

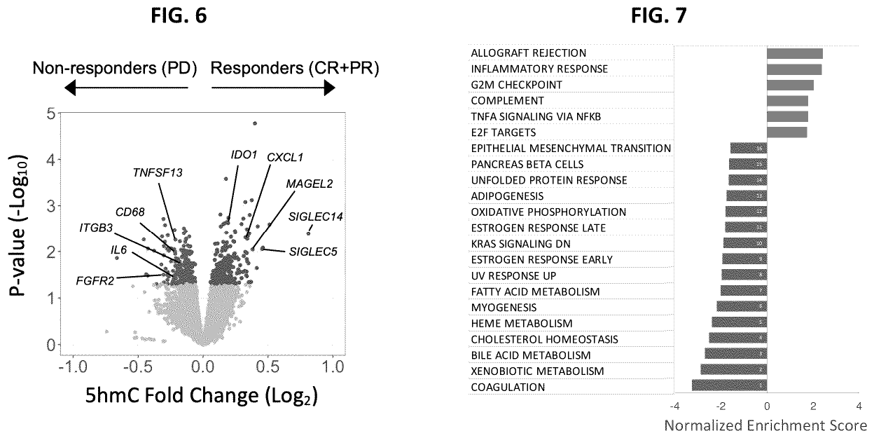

To determine whether responders had distinct 5hmC profiles of plasma cfDNA relative to non-responding patients, plasma-derived 5hmC profiles at baseline timepoints (prior to start of treatment) were compared. Patients who had progressive disease as determined by radiologic imaging according to RECIST 1.1 criteria were categorized as non-responders, whereas complete and partial responders were grouped as responders. This differential 5hmC analysis identified 482 genes with p-value<below 0.05 (FIG. 6). Among the genes with increased 5hmC in responders relative to non-responders, 5hmC was higher over genes with known functions in immune response such as sialic acid-binding immunoglobulin-like lectin 5 (SIGLEC5) (Vuchkovska et al. (2022) “Siglec-5 is an inhibitory immune checkpoint molecule for human T cells,” Immunology) and CXCL1 (FIG. 6). On the other hand, non-responders had elevated 5hmC over genes associated with metastasis/epithelial mesenchymal transition or resistance to anti-PD-1 therapy such as FGFR2 (Ranieri, D. et al. (2015) “Expression of the FGFR2 mesenchymal splicing variant in epithelial cells drives epithelial-mesenchymal transition,” Oncotarget 7:5440-5460) and IL6 (Huseni, M. A. et al. (2023) “CD8+ T cell-intrinsic IL-6 signaling promotes resistance to anti-PD-L1 immunotherapy,” Cell Reports Medicine 4:100878). (FIG. 6).

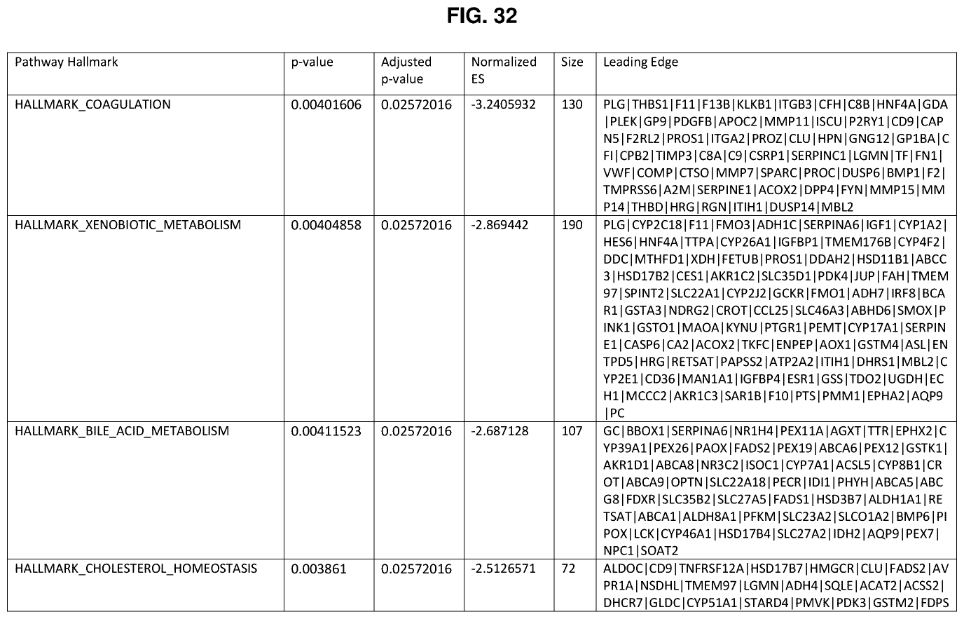

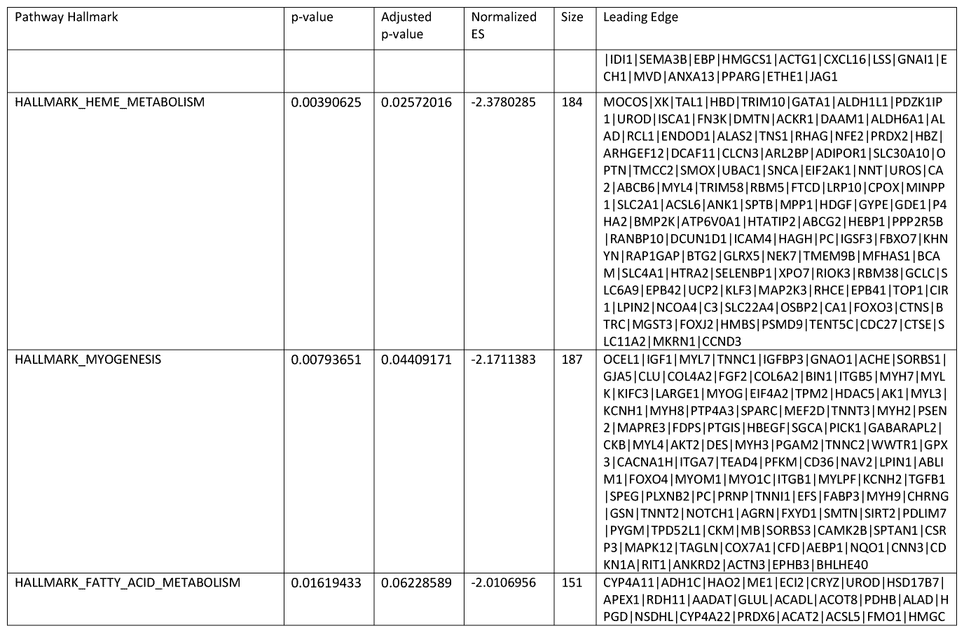

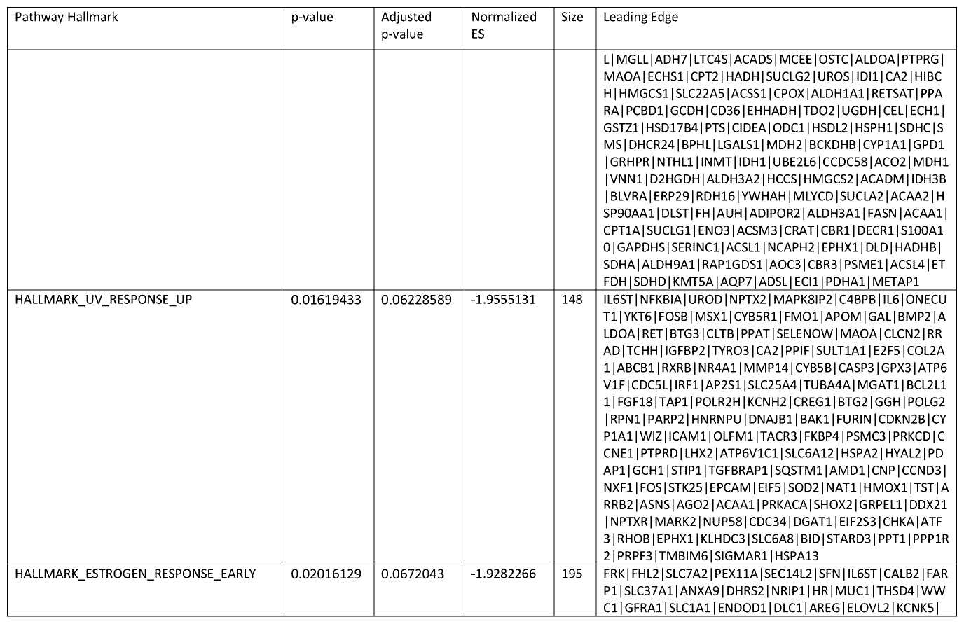

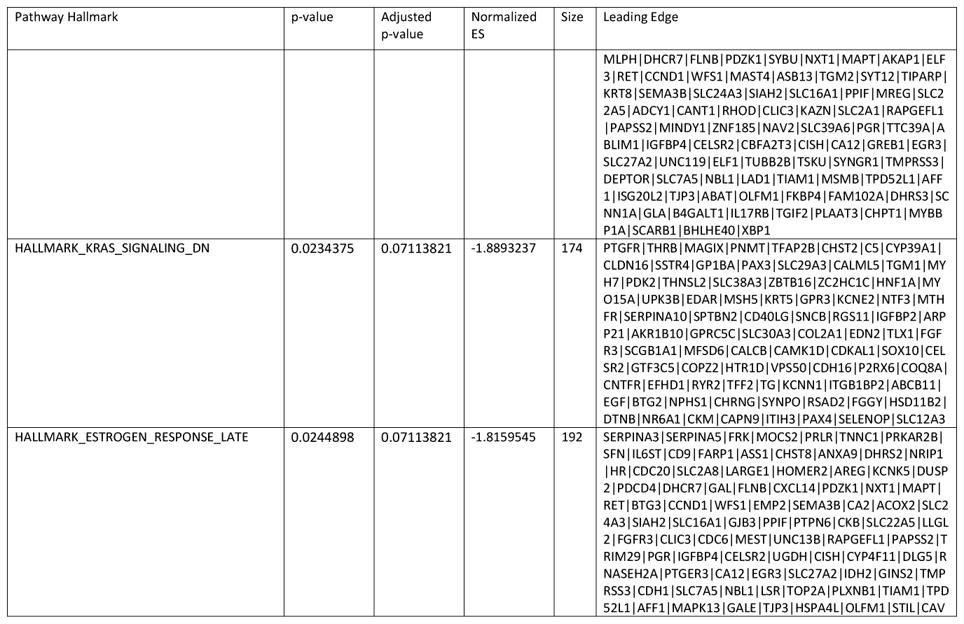

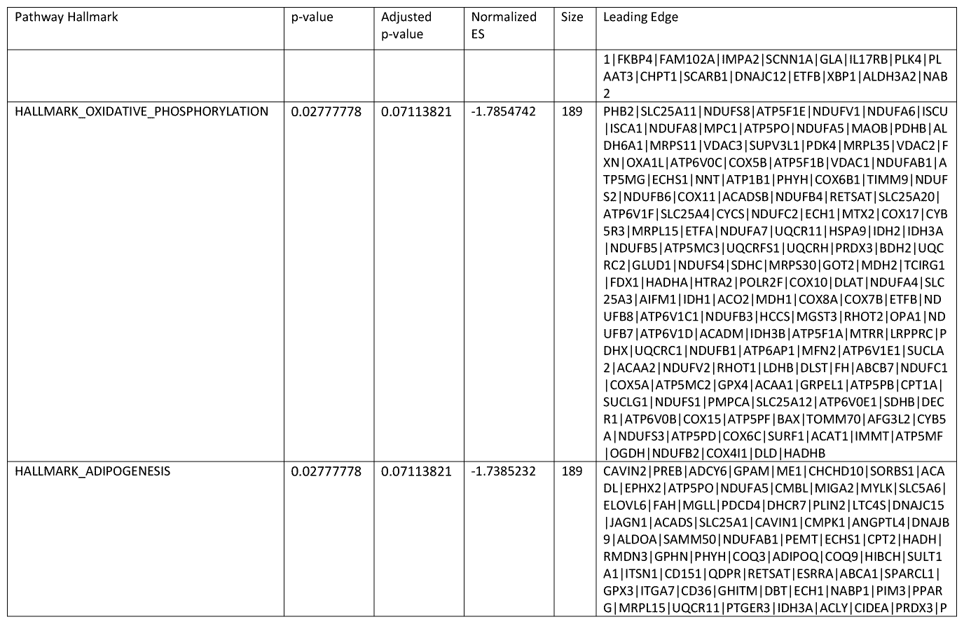

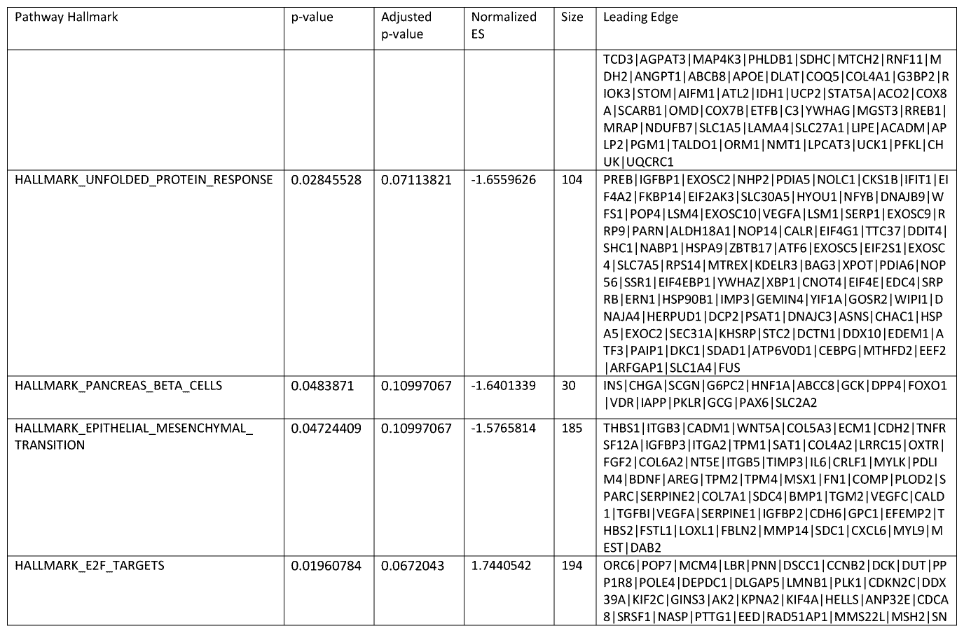

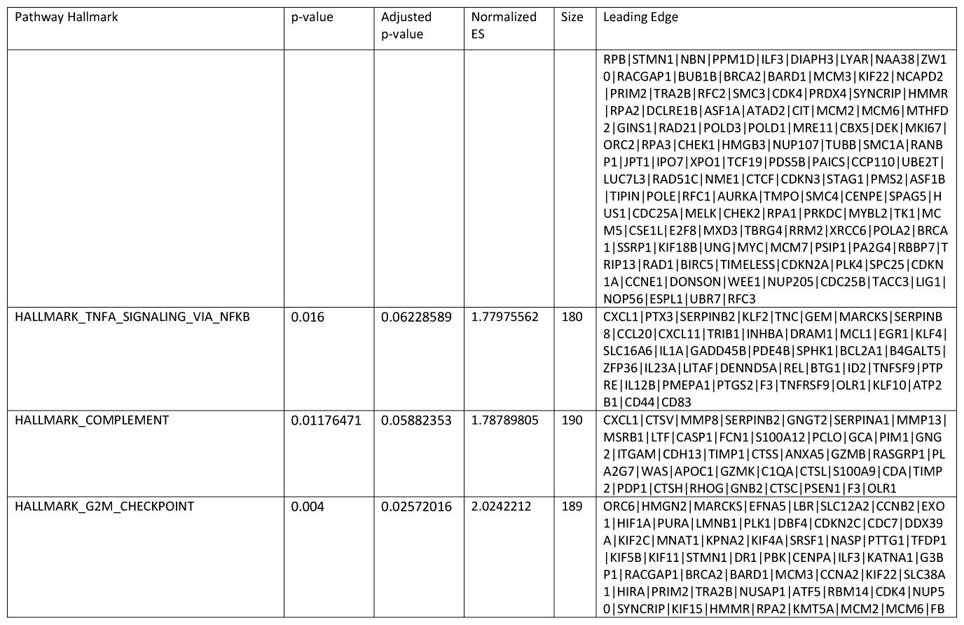

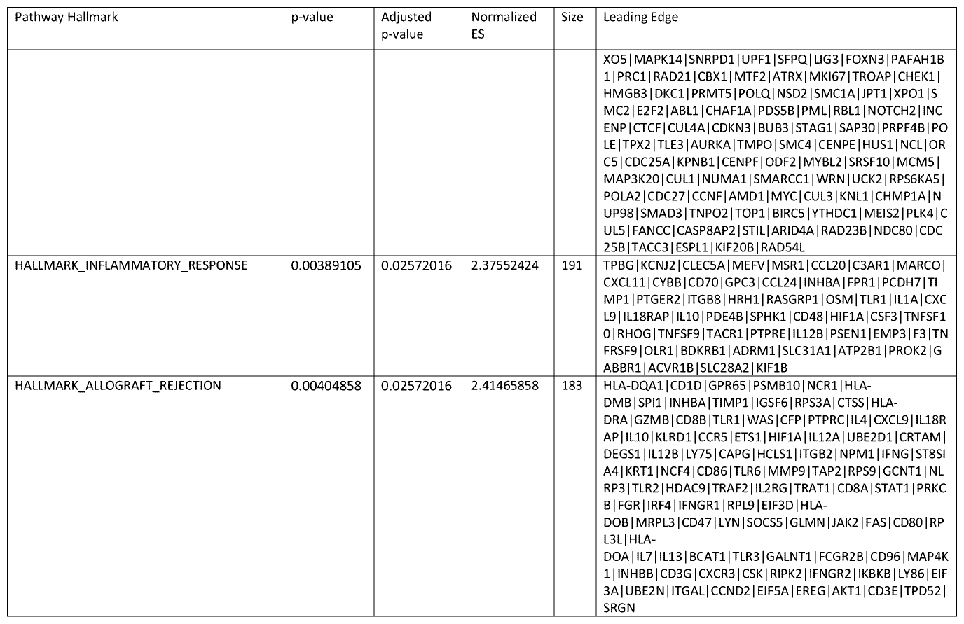

Next, gene set enrichment analysis (GSEA) was performed to identify biological processes that can be distinguished by comparing pre-treatment cfDNA 5hmC profiles in responding patients to pre-treatment cfDNA 5hmC profiles in non-responders. This analysis revealed several gene sets that are associated with an inflamed immune state such as allograft rejection, inflammatory response and TNFa signaling via NF-κB to be upregulated in responders relative to non-responders (FIG. 7). The most significantly enriched pathways, as determined by GSEA, associated with response to immunotherapy (as evidenced by an increased 5hmC level in responders) were identified as the following (also see FIG. 7 and FIG. 32):
HALLMARK_ALLOGRAFT_REJECTION;
HALLMARK_INFLAMMATORY_RESPONSE;
HALLMARK_G2M_CHECKPOINT;
HALLMARK_COMPLEMENT;
HALLMARK_TNFA_SIGNALING_VIA_NFKB; and
HALLMARK_E2F_TARGETS.

The following 16 gene sets were found to be enriched in non-responders relative to responders as determined by GSEA (FIG. 7 and FIG. 32):
HALLMARK_COAGULATION;
HALLMARK_XENOBIOTIC_METABOLISM;
HALLMARK_BILE_ACID_METABOLISM;
HALLMARK_CHOLESTEROL_HOMEOSTASIS;
HALLMARK_HEME_METABOLISM;
HALLMARK_MYOGENESIS;
HALLMARK_FATTY_ACID_METABOLISM;
HALLMARK_UV_RESPONSE_UP;
HALLMARK_ESTROGEN_RESPONSE_EARLY;
HALLMARK_KRAS_SIGNALING_DN;
HALLMARK_ESTROGEN_RESPONSE_LATE;
HALLMARK_OXIDATIVE_PHOSPHORYLATION;
HALLMARK_ADIPOGENESIS;
HALLMARK_UNFOLDED_PROTEIN_RESPONSE;
HALLMARK_PANCREAS_BETA_CELLS; and
HALLMARK_EPITHELIAL_MESENCHYMAL_TRANSITION.

Notably, among the genesets that were upregulated in non-responders were epithelial mesenchymal transition genes, which had previously been associated with resistance to anti-PD-1 treatment. See Mariathasan et al. (2018), “TGFβ attenuates tumour response to PD-L1 blockade by contributing to exclusion of T cells,” Nature 554:544-548. Furthermore, genes that are downregulated upon KRAS activation were downregulated in responding patients, suggesting KRAS activation in responders. Interestingly, KRAS mutations had previously been correlated with response and better outcomes upon immunotherapy treatment. See Liu et al. (2020), “The superior efficacy of anti-PD-1/PD-L1 immunotherapy in KRAS-mutant non-small cell lung cancer that correlates with an inflammatory phenotype and increased immunogenicity,” Cancer Lett 470:95-105; and Lauko et al. (2021), “Impact of KRAS mutation status on the efficacy of immunotherapy in lung cancer brain metastases,” Sci Rep-uk 11:18174.

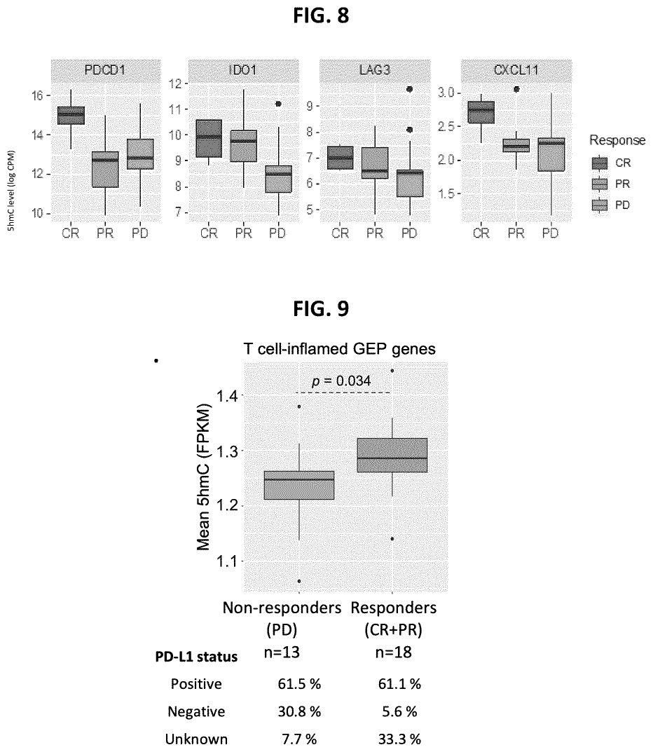

Previous RNA analysis of tissue samples obtained from patients who received anti-PD-1 treatment had revealed a T cell-inflamed gene expression profile (GEP) that incorporates RNA abundance for 18 genes to distinguish responders from non-responders (see Ayers et al. (2017) and Cristescu et al. (2018), cited previously). Given that the preponderance of the differentially hydroxymethylated genes are known to be associated with immune response and drug resistance, the 5hmC levels over these previously defined genes within the T cell-inflamed GEP was evaluated next. Notably, a number of genes that are important in modulating T cells, such as PDCD1 (which encodes PD-1 protein), IDO-1, LAG-3 and CXCL11, were differentially hydroxymethylated (FIG. 8). Furthermore, mean 5hmC levels of T cell-inflamed GEP genes were significantly higher in responders compared to non-responders (FIG. 9). The previous work alluded to above demonstrated that increased expression of these 18 T cell-inflamed GEP genes in tumor tissue and in the tumor microenvironment can be used to predict immunotherapy response, but the work did not relate to 5hmC levels in plasma cfDNA.

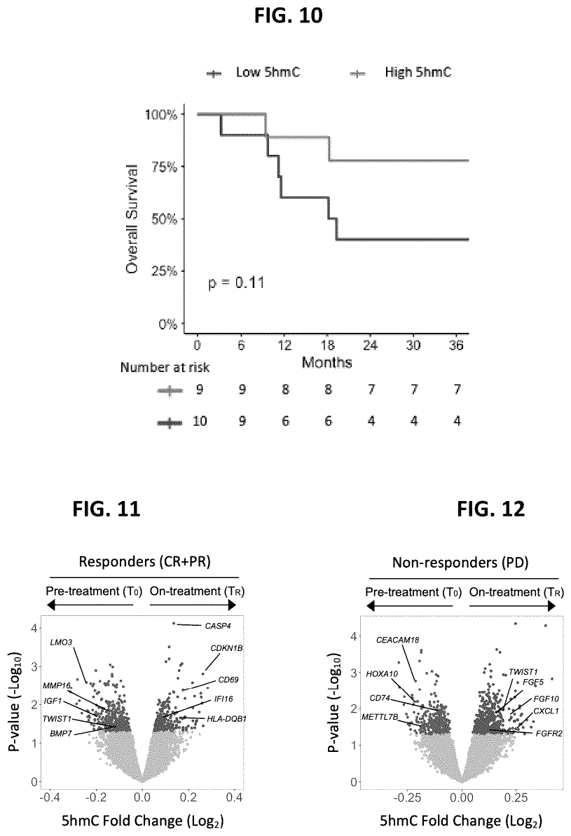

We evaluated 5hmC levels in plasma cfDNA over the 18 T-cell inflamed GEP genes to see whether they could be distinguish those patients with better overall survival. A subset of patients had follow-up for a minimum of 36 months post-treatment start (total n=19). For survival analysis, these patients were separated into two groups according to their mean 5hmC values over the 18 T-cell inflamed GEP genes, where patients above or below the median of the full cohort were assigned to high 5hmC (n=9) or low 5hmC (n=10) groups, respectively. As expected, based on the results that showed higher 5hmC levels over the T-cell inflamed GEP genes for responders relative to non-responders (FIG. 9), a trend separating the two groups in terms of overall survival was observed, however it did not reach statistical significance with a p-value of 0.11 in this subset of patients (FIG. 10). Overall, the results show that the expression of these tissue-relevant genes in predicting therapy response can also be captured by increased hydroxymethylation using the present plasma cfDNA analysis without the need for tissue samples. Altogether, these findings indicate that immune relevant signatures consistent with immunotherapy response can be identified in plasma cfDNA using 5hmC profiles.

(iv) Differential 5-Hydroxymethylation in cfDNA Upon Anti-PD-1 Treatment in Responding and Non-Responding Patients:

To examine whether there were any changes in plasma-derived 5hmC profiles after anti-PD-1 treatment, paired differential analysis was performed using 5hmC fragment counts per gene by comparing the timepoint of radiologic response (T_{R}) to baseline timepoint that was collected before treatment start (T₀). Patients who had progressive disease, as determined by radiologic imaging according to RECIST 1.1 criteria, were categorized as non-responders, whereas complete and partial responders were grouped as responders. This differential analysis yielded 530 genes with a p-value<0.05 in responders (FIG. 11). Among the genes with increased 5hmC counts were genes associated with tumor immunity or immune cell activation such as HLA-DQB1 and CD69, while the genes with decreased 5hmC counts were enriched for cancer or drug resistance genes such as IGF1, TWIST1 and MMP16. In non-responding patients, a similar comparison of on-treatment samples from time of radiologic response (T_{R}) to paired pre-treatment (TO) samples identified 715 genes with differential 5hmC (FIG. 12). In contrast to responding patients, genes with increased 5hmC included genes that are associated with resistance to checkpoint inhibition based on previous studies of tissue samples, such as FGF signaling (Adachi et al. (2021) “Inhibition of FGFR Reactivates IFNγ Signaling in Tumor Cells to Enhance the Combined Antitumor Activity of Lenvatinib with Anti-PD-1 Antibodies,” Cancer Res 82:292-306), while genes with decreased 5hmC included CD74 with known functions in immune response (Su et al. (2017) “The biological function and significance of CD74 in immune diseases,” Inflamm. Res. 66, 209-216).

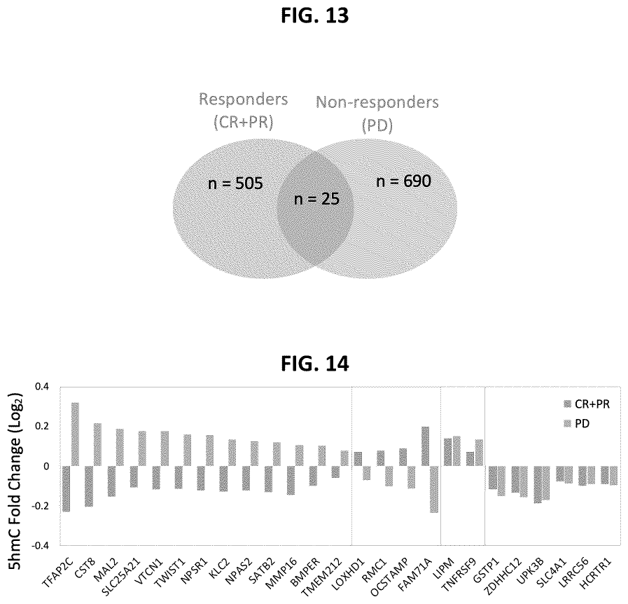

Among the genes differentially hydroxymethylated after treatment, only 25 genes were shared between responders and non-responders (FIG. 13). The majority of these genes (17 out of 25) displayed opposite trends between responders and non-responders (FIG. 14). Genes that increased in 5hmC in non-responders while decreasing in responders included multiple cancer-associated genes such as MMP16 and TWIST1. Altogether, the minimal overlap between the differentially hydroxymethylated genes in non-responder and responder cohorts suggests that these changes detected in plasma cfDNA after treatment are specific to anti-PD-1 response/resistance.

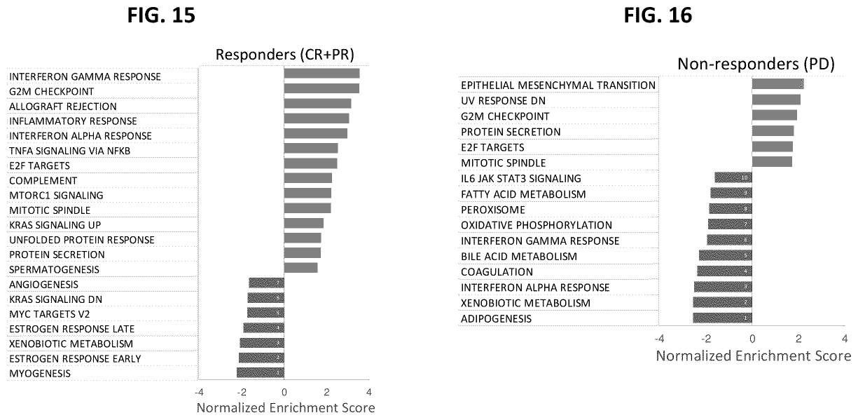

To determine whether 5hmC profiles in plasma-derived cfDNA can inform about the biological response to anti-PD-1 treatment, we performed gene set enrichment analysis (GSEA) using 5hmC counts over gene bodies. In responders, the top enriched pathways were heavily immune-related; such as interferon gamma (IFNg) response, inflammatory response, interferon alpha (IFNa) response and TNFa signaling via NFkB (FIG. 15). IFNg response, the most significantly enriched pathway in 5hmC analysis of plasma cfDNA, was previously associated with anti-PD-1 response by gene expression analysis of tumor tissue samples taken at baseline from patients who went on to be treated with pembrolizumab (see Ayers et al. (2017) and Cristescu et al. (2018), cited earlier herein). For non-responding patients, there were fewer gene sets that significantly changed upon treatment. Among the top enriched gene sets identified in non-responders was epithelial mesenchymal transition (EMT) gene set (FIG. 16), which is known to be associated with resistance to several drugs including ICIs. See Mariathasan et al. (2018), “TGFβ attenuates tumour response to PD-L1 blockade by contributing to exclusion of T cells,” Nature 554:544-548. Hugo, W. et al. (2016) “Genomic and Transcriptomic Features of Response to Anti-PD-1 Therapy in Metastatic Melanoma.” Cell 165, 35-44. These findings indicate that inflamed T cell and EMT signatures found in the tumor microenvironment of responders and non-responders, respectively, can be identified in plasma cfDNA by 5hmC analysis.

(v) Differentially Hydroxymethylated Loci Identified in cfDNA in Distinguishing Normal Lung Tissue from Lung Cancer Tissue:

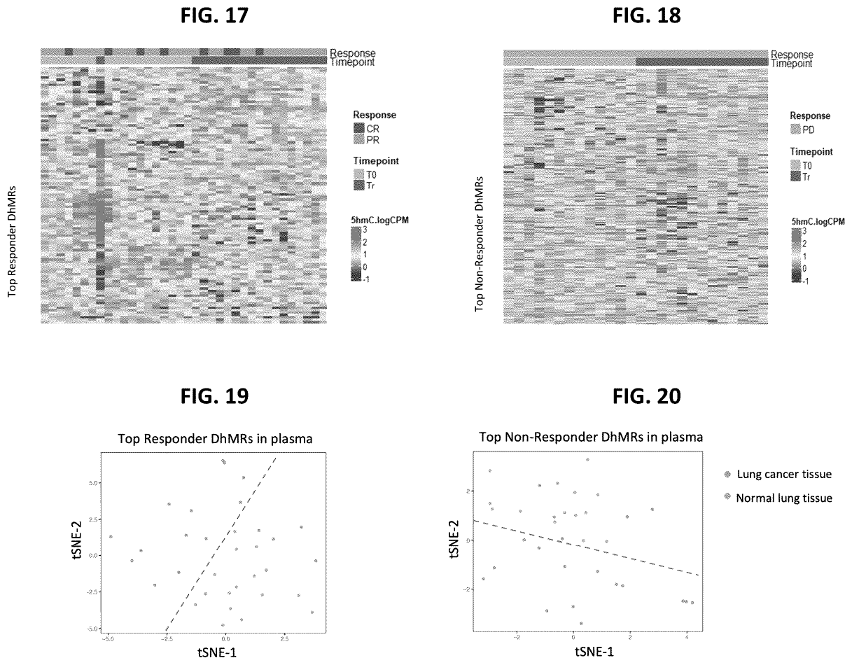

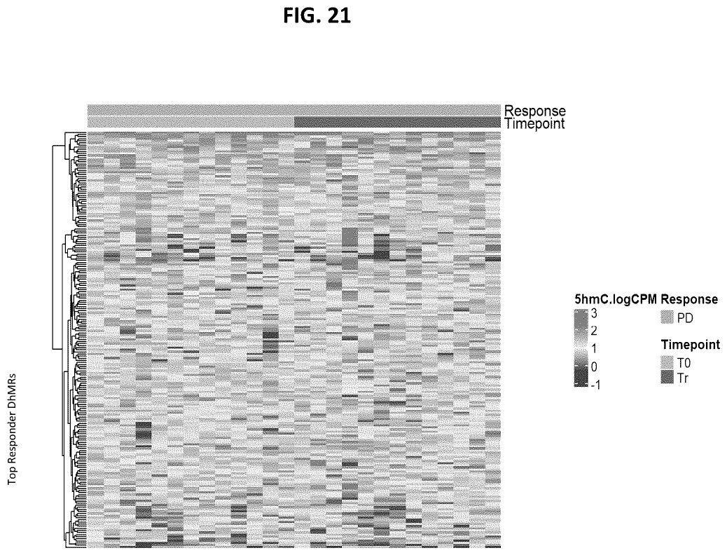

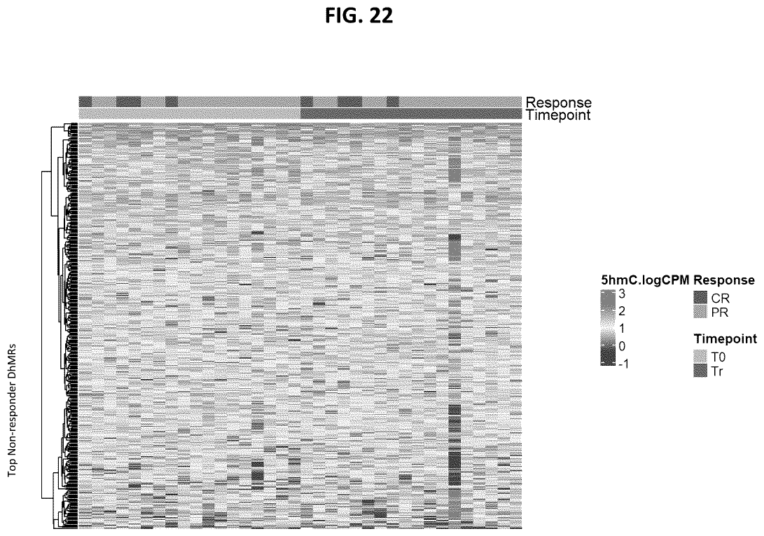

Differential hydroxymethylated genes in cfDNA identified in this study have previously been associated with ICI response and resistance in tissue transcriptome analysis. Therefore, we sought to ask whether the changes observed in cfDNA hydroxymethylome are at least partially derived from the hydroxymethylome of tumor-derived DNA. For this purpose, we first identified the differentially hydroxymethylated regions (DhMRs) upon anti-PD-1 treatment by comparing 5hmC profiles at T_{Q} to T₀ in responders and in non-responders. 152 and 301 DhMRs with a p-value below 0.05 and a fold change exceeding 1.5 were identified by Wilcoxon rank-sum test in responders (FIG. 17) and non-responders (FIG. 18), respectively. These DhMRs which were separately identified in responders and non-responders were specific and non-overlapping (FIG. 21 and FIG. 22). T₀ investigate whether these DhMRs identified in plasma were informative with regards to 5hmC profile changes in tumor tissue, 5hmC profiles were generated for 15 normal lung tissues and 18 lung cancer tissues. Both top responder (FIG. 17) and non-responder (FIG. 18) DhMRs identified in plasma were able to distinguish between tumor lung tissue and normal lung tissue as visualized by t-distributed stochastic neighbor embedding (t-SNE) (FIG. 19 and FIG. 20). These results suggest that the DhMRs identified in plasma are congruent with changes in 5hmC profiles of lung tumor tissue.

(vi) Anti-PD-1 Therapy Response Monitoring with Plasma-Derived 5hmC Profiles:

The observation that the anti-PD-1 treatment induced 5hmC changes were significantly non-overlapping between responders and non-responders indicates that such regions can be used to monitor treatment response. To test this idea, first, genomic loci with the most significant changes between the responding and non-responding cohorts were determined by calculating treatment-induced fold change in 5hmC occupancy normalized to baseline ((T_{R}−T₀)/T₀) (i.e., ζ, as defined previously) and then identifying the regions with the most statistically significant changes by applying a threshold p-value of 0.05 and a difference ζ of at least 1.5. This resulted in identification of 154 regions with decreased 5hmC and 129 regions with increased 5hmC in responders relative to non-responders.

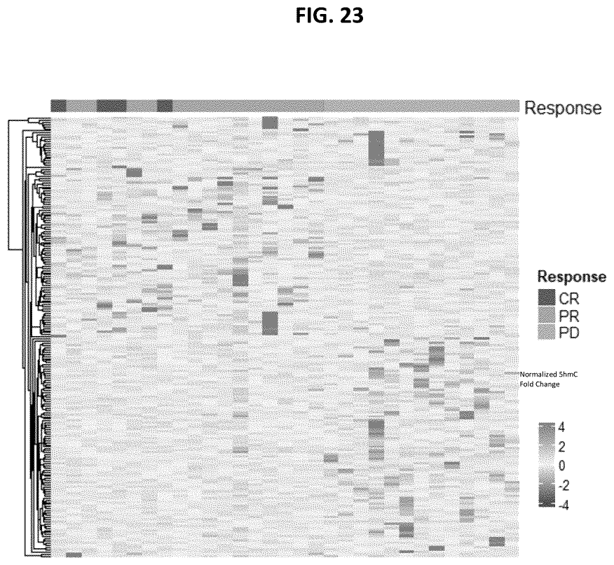

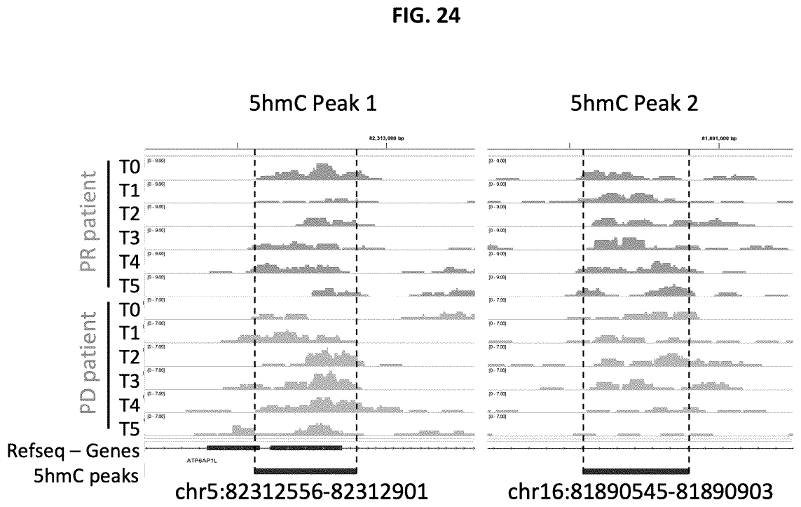

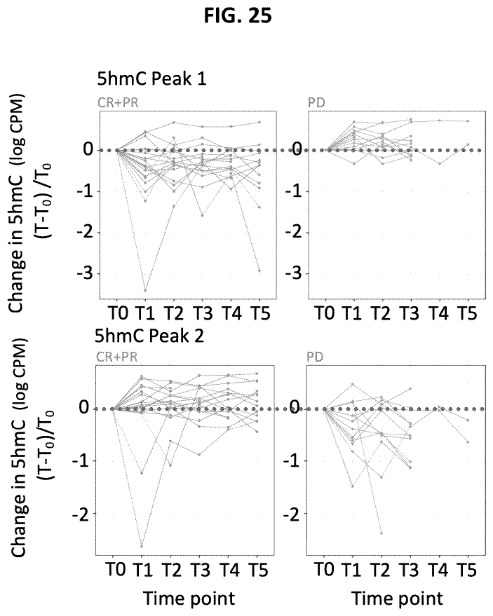

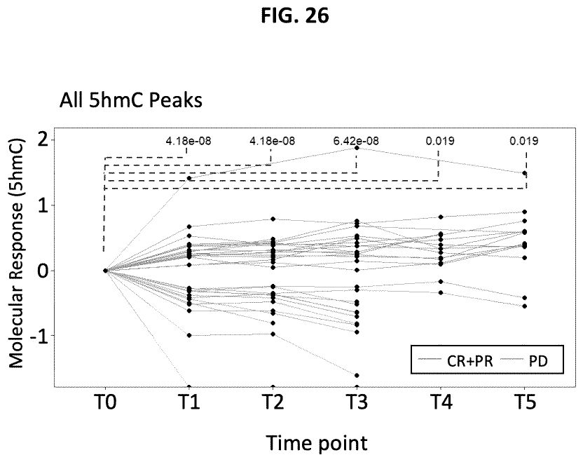

The majority of these loci mapped to gene features as visualized for two representative 5hmC peak loci using the genome browser IGV (FIG. 24). Examination of all study timepoints showed that the changes in 5hmC levels observed at the time of radiological response can be detected as early as 4-6 weeks from therapy start (FIG. 25). Based upon this observation, the molecular response elicited by anti-PD-1 treatment on the plasma hydroxymethylome was evaluated, as measured by change in 5hmC counts over the top differential regions whose hydroxymethylation levels correlated either negatively or positively with response as identified in FIG. 23. The differential molecular response based on 5hmC levels could distinguish responders from non-responders as early as the first time point, which was 4-6 weeks from therapy start (FIG. 26). These results show that 5hmC profiles obtained from plasma contain treatment induced changes that could serve as molecular response markers with potential for earlier detection than radiologic response. Altogether, our findings demonstrate that 5hmC-based changes identified in plasma samples of NSCLC patients can be utilized for anti-PD-1 therapy response monitoring.

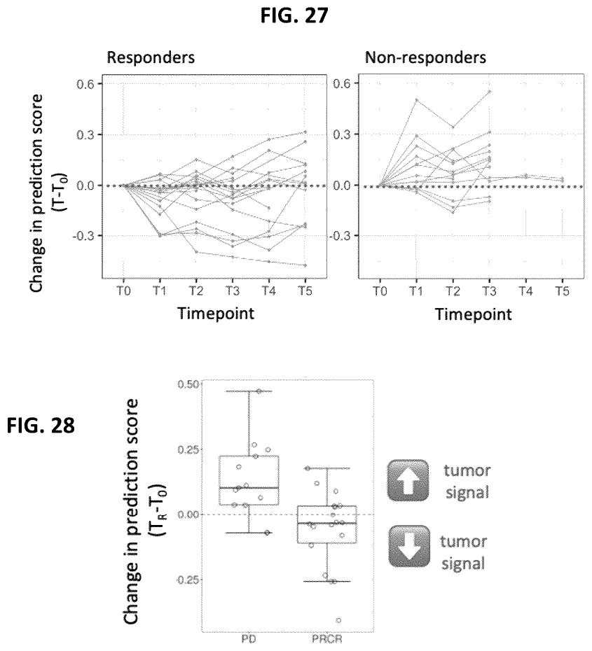

To test whether 5hmC profiles can be used as a proxy for tumor burden to assess therapy response, a predictive model was built using lung cancer samples to classify plasma samples from patients with early stage disease and lower tumor burden and patients with late stage advanced disease with higher tumor burden. The training set used to build this prediction model was distinct and did not overlap with any of the patients in the anti-PD-1 treatment cohorts. When this model was applied to score the plasma samples from patients who received anti-PD-1 treatment but did not respond (non-responders), prediction scores for on-treatment plasma samples increased relative to prediction scores for the baseline plasma sample collected from the same patient (FIG. 27, left panel). On the other hand, prediction scores for on-treatment plasma samples dropped relative to baseline for majority of the patients who were in the responder group (FIG. 27, right panel). Comparison of prediction scores at time of radiologic response to baseline revealed that for 73% of the patients, 5hmC profiles could predict change in tumor burden consistently with RECIST (FIG. 28). Altogether, our results suggest that 5hmC-based biomarkers can be observed in plasma samples of NSCLC patients for anti-PD-1 therapy response monitoring.

Immune checkpoint inhibitors can exert significant and durable anti-tumor effects, albeit in a subpopulation of patients. In the above experiments, the potential was explored for utilizing 5hmC-based changes that are detectable in plasma-derived cfDNA to predict and monitor anti-PD-1 treatment response in NSCLC patients. The results indicate that cfDNA 5hmC profiles contain loci that are correlated with anti-PD-1 response that could be exploited for biomarker discovery for patient selection and therapy response monitoring for immune checkpoint inhibitors in treatment of lung cancer.

Among the patient selection biomarkers that are currently used in the clinic is PD-L1 immunohistochemistry using tumor biopsy samples. Yet, it has been reported that patients with PD-L1 immunostaining below established thresholds may still benefit from anti-PD-1 treatments. Furthermore, lack of response is also observed in PD-L1 positive patients (Wang et al. (2021), “Toward personalized treatment approaches for non-small-cell lung cancer,” Nat Med 27:1345-1356, Gibney (2016), “Predictive biomarkers for checkpoint inhibitor-based immunotherapy,” Lancet Oncol 17: e542-e551). Both intra- and inter-tumor heterogeneity with respect to PD-L1 expression may yield such contradictory results as tumor sampling at a single tumor site might not accurately reflect the state of overall PD-L1 expression in a patient's tumor(s). A second important variable is the poor uniformity in the PD-L1 immunohistochemistry antibodies and different thresholds for PD-L1 positivity used (Gibney (2016), “Predictive biomarkers for checkpoint inhibitor-based immunotherapy,” Lancet Oncol 17: e542-e551). These limitations can be circumvented by the method provided herein, which captures DNA shed not only by cancer cells from different tumor sites but also by tumor microenvironment and immune cells. In particular, analysis of plasma cfDNA 5hmC, which marks active biological pathways, can provide better understanding of drug response in tumor and immune cells using a single analyte. Indeed, the inventors herein have found that plasma cfDNA 5hmC profiles are significantly different in responders relative to non-responders both prior to treatment (FIGS. 11-16) as well as after treatment (FIGS. 6-9). The present analysis revealed 5hmC enrichment over immune relevant genes, indicating increased immune gene activation in responders. Given that 60% of both cohorts were PD-L1 positive (FIG. 2), these results show that 5hmC profiling can provide an independent measurement of immune status that is informative of potential response to immunotherapy.

Plasma-derived cfDNA analysis as provided herein presents unique advantages as a source for candidate biomarkers for ICI response and treatment monitoring. Non-invasive biomarkers circumvent the challenges related to tumor biopsies, such as low rates of patient compliance, the potential for complications and insufficient material. In addition, non-invasive biomarkers can facilitate serial sampling, which allows monitoring of dynamic changes during treatment which can be related to immune activation or acquired resistance. In this study, anti-PD-1 therapy response induced distinct changes in plasma cfDNA 5hmC profiles of responders compared to non-responders and were observed as early as 4-6 weeks following the onset of treatment (FIGS. 25-26). Our results show that 5hmC analysis offers a novel non-invasive method for serial monitoring of anti-PD-1 therapy response in plasma.

Plasma-derived cfDNA is a pool of DNA that arises from multiple tissue sources. While immune cells make up the majority of cfDNA, particularly in healthy subjects, tumor cells and other components of the tumor microenvironment also shed DNA into blood in cancer patients. Methods confined to analysis of tumor-derived cfDNA, such as tumor-informed mutation analysis, not only necessitate addressing sensitivity challenges resulting from tumor-derived DNA dilution in a cfDNA sample, but also fail to provide information on the tumor microenvironment. Tissue specific marks in cfDNA, particularly 5hmC as described herein, help address the challenges associated with the cfDNA heterogeneity. Consistently, our 5hmC analysis of cfDNA identified IFN response, inflammatory response in responders, and epithelial mesenchymal transition in non-responders, pointing to both immune cell-derived and tumor cell-derived biology.

1. A method for monitoring a patient with a lung cancer during lung cancer therapy to determine efficacy of the therapy, the method comprising:
(a) obtaining a baseline hydroxymethylation signature for a lung cancer patient prior to receiving a lung cancer therapy by: (i) obtaining a cell-free DNA (cfDNA) sample from the patient, enriching for hydroxymethylated DNA in the sample, amplifying the hydroxymethylated DNA, and sequencing the amplified hydroxymethylated DNA in a manner that identifies 5-hydroxymethylcytosine (5hmC)-containing fragments or sites in the DNA; and (ii) measuring hydroxymethylation levels in the sequenced cfDNA at each of a plurality of hydroxymethylation biomarker loci, wherein each hydroxymethylation biomarker locus is selected as exhibiting an increase or decrease in hydroxymethylation in a manner that correlates with the presence of lung cancer;
(b) using the baseline hydroxymethylation signature as a first input parameter to a computer-generated predictive model comprising a trained machine learning model, thereby providing a first probability score;
(c) obtaining a monitoring hydroxymethylation signature for the lung cancer patient by repeating the process of (a) during treatment of the patient with the lung cancer therapy;
(d) using the monitoring hydroxymethylation signature as a second input parameter to the computer-generated predictive model to provide a second probability score; and
(e) comparing the second probability score to the first probability score to derive a differential probability score characterizing a likelihood that the patient is responding to the lung cancer therapy.

2. The method of claim 1, wherein when the second probability score is greater than the first probability score, determining that the lung cancer therapy is ineffective.

3. The method of claim 1, wherein each hydroxymethylation biomarker locus is selected as exhibiting an increase or decrease in hydroxymethylation in a manner that correlates with response to immunotherapy.

4. The method of claim 1, wherein each hydroxymethylation biomarker locus is selected as exhibiting an increase or decrease in hydroxymethylation in a manner that correlates with lung cancer tumor load.

5. The method of claim 1, wherein the baseline probability score and the monitoring probability score are calculated using a logistic regression analysis of the differences in hydroxymethylation level at each of the hydroxymethylation biomarker loci.

6. The method of claim 1, wherein each hydroxymethylation biomarker locus is selected as exhibiting differential hydroxymethylation as determined by a Wilcoxon rank-sum test with a p-value of less than 0.05 and a fold change of at least 1.5 between lung cancer patients who do not respond to the lung cancer therapy and lung cancer patients who do respond to the lung cancer therapy.

7. The method of claim 1, wherein (e) further comprises combining the differential probability score with an additional feature value for at least one additional feature type to characterize the likelihood that the patient is responding to the lung cancer therapy.

8. The method of claim 7, wherein the additional feature type comprises DNA fragment size distribution, copy number variation, cfDNA concentration, methylation profile, T-cell-inflamed gene expression profile, circulating tumor DNA count, serum CA19-9 level, serum CA125 level, LAG3 expression, IDO-1 expression, T-cell count, inflammation gene signature, myeloid-derived suppressor cell count, lymphocyte count, deficient mismatch repair, tumor mutational burden, presence or absence of germline mutations, a patient-specific clinical parameter, and combinations of any of the foregoing.

9. The method of claim 7, wherein the additional feature type comprises:
number of cfDNA fragments in each of at least two nonoverlapping size ranges;
copy number variation in the cfDNA sample;
concentration of cfDNA in the cfDNA sample;
a patient-specific clinical parameter; and
combinations of any of the foregoing.

10. The method of claim 9, wherein the patient-specific clinical parameter is selected from lesion size; lesion grade; lesion stage; lesion location; patient age; patient weight; patient gender; patient ethnicity; cigarette smoking status; and exposure or lack of exposure to a known carcinogen.

11. The method of claim 7, wherein the combining comprises an ensemble analysis.

12. The method of claim 11, wherein the ensemble analysis is a stacked ensemble analysis.

13. The method of claim 2, further comprising, after determining that the lung cancer therapy is ineffective, discontinuing the lung cancer therapy.

14. The method of claim 13, further including changing to a different lung cancer therapy.

15. The method of claim 14, wherein the different lung cancer therapy comprises administration of a higher dose of medication, administration of a different medication, or altering treatment modality.

16. The method of claim 15, wherein the different lung cancer therapy is determined using the baseline hydroxymethylation profile, the monitoring hydroxymethylation profile, or both the baseline hydroxymethylation profile and the monitoring hydroxymethylation profile.

17. The method of claim 1, wherein responding to immunotherapy comprises exhibiting a partial response, a complete response, or stable disease as defined in RECIST guidelines 1.1.

18. The method of claim 1, wherein the lung cancer is non-small cell lung cancer.

19. The method of claim 1, wherein the lung cancer is selected from adenocarcinomas, squamous cell carcinomas, small-cell lung carcinomas, adenosquamous carcinomas, carcinoid tumors, bronchial gland carcinomas, and sarcomatoid carcinomas.

20. The method of claim 1, wherein the lung cancer therapy is an immunotherapy.

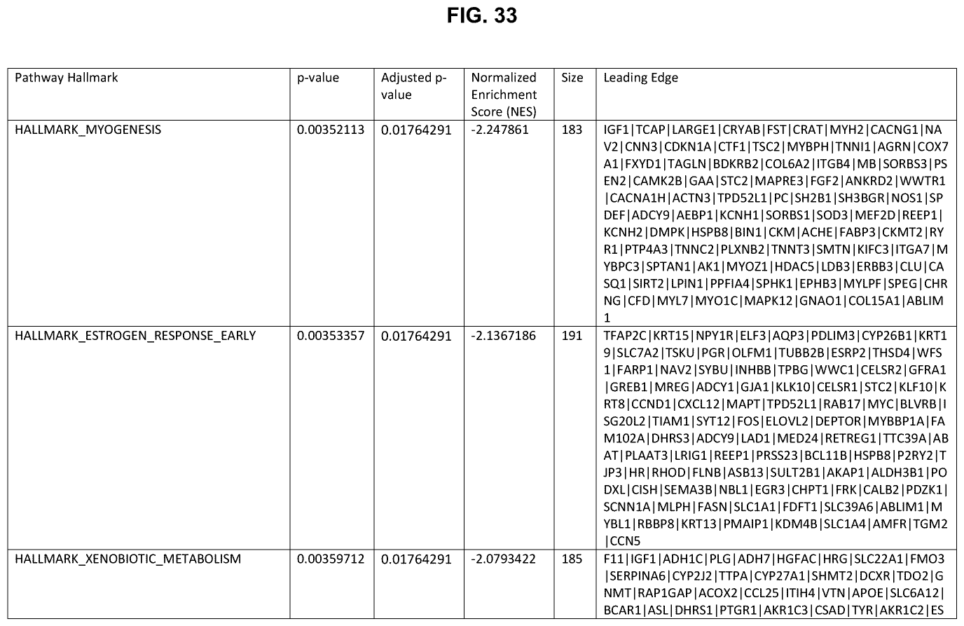

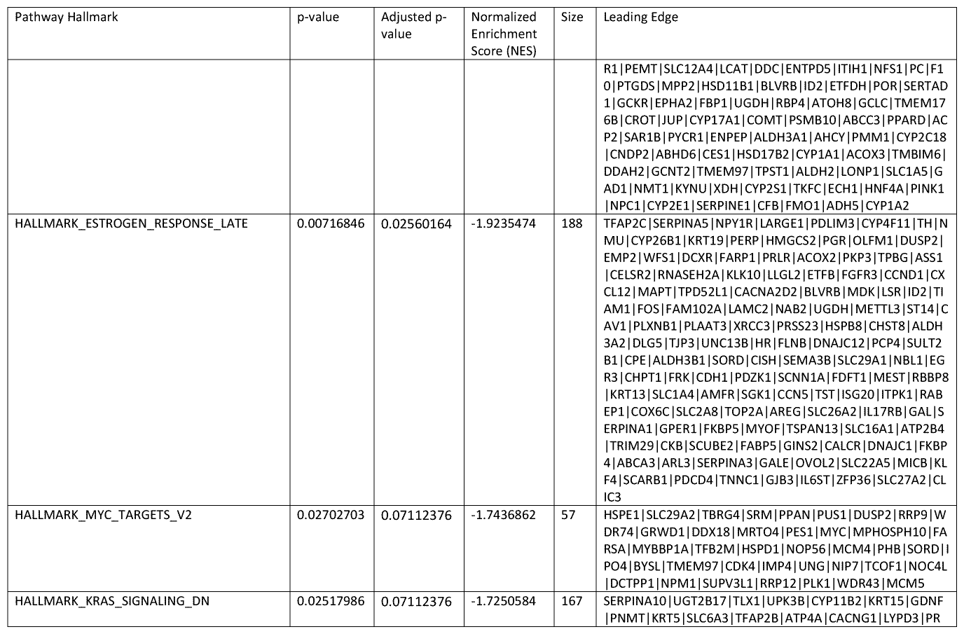

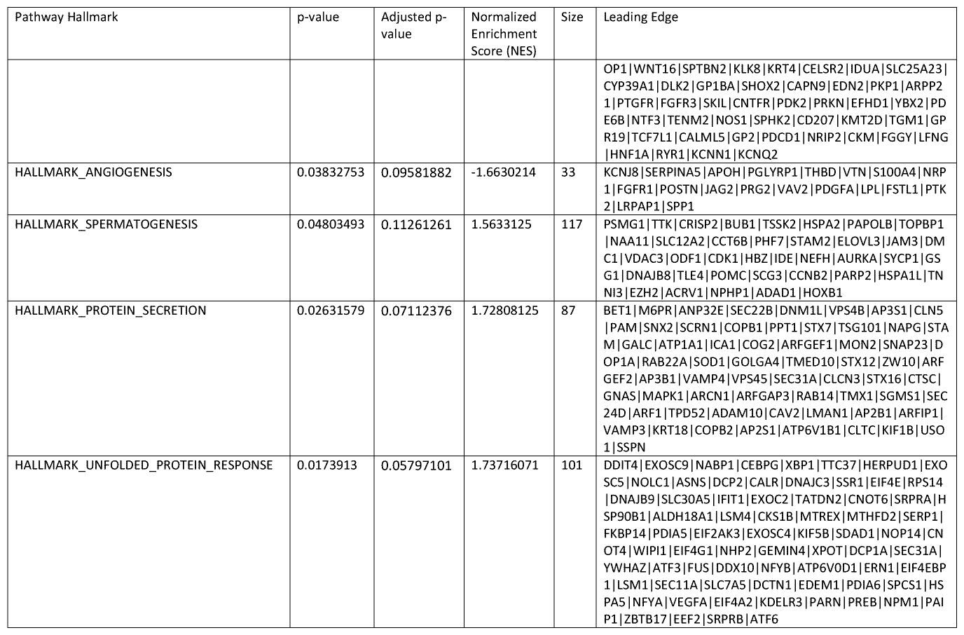

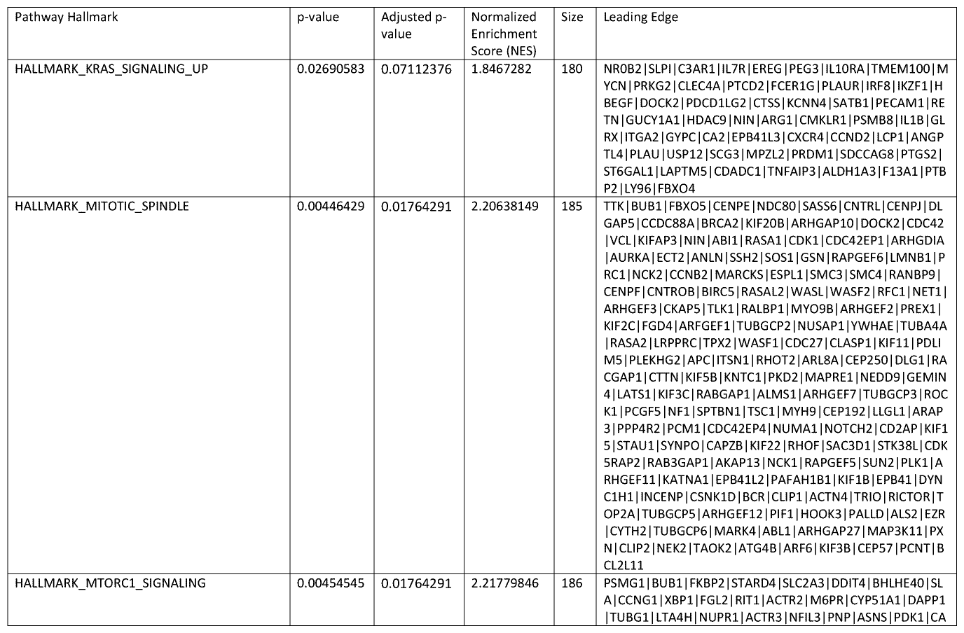

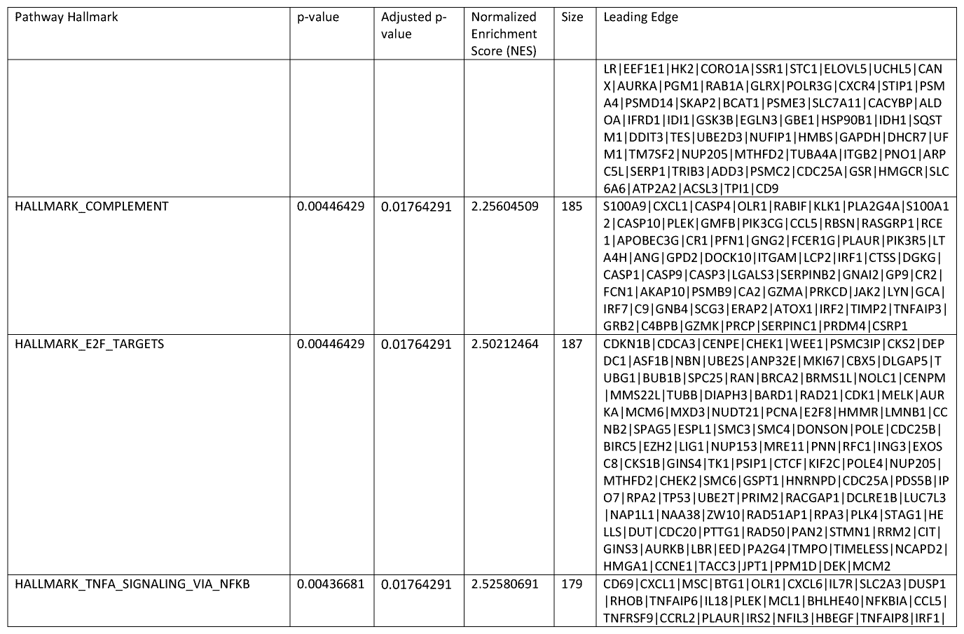

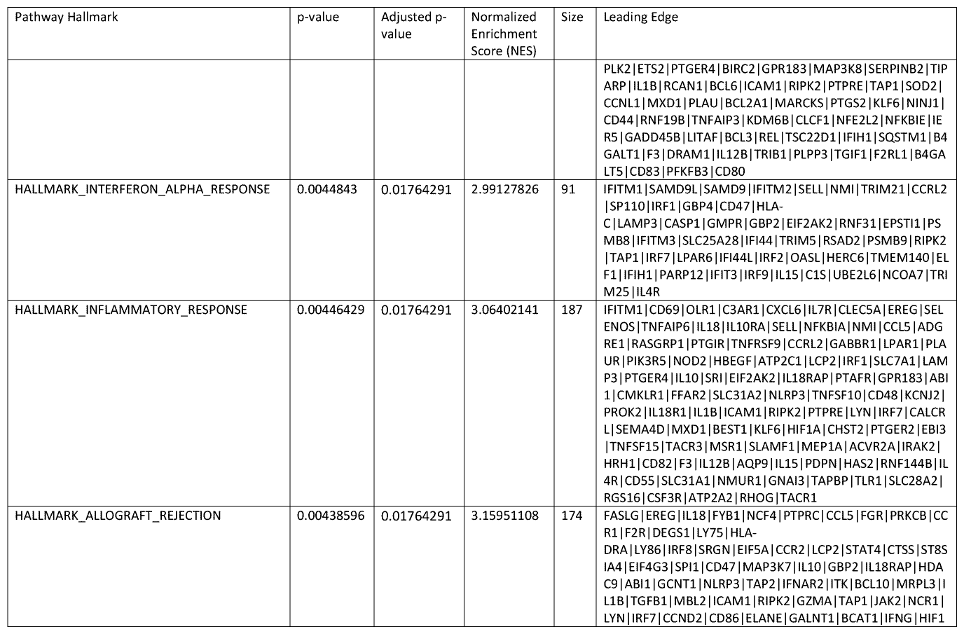

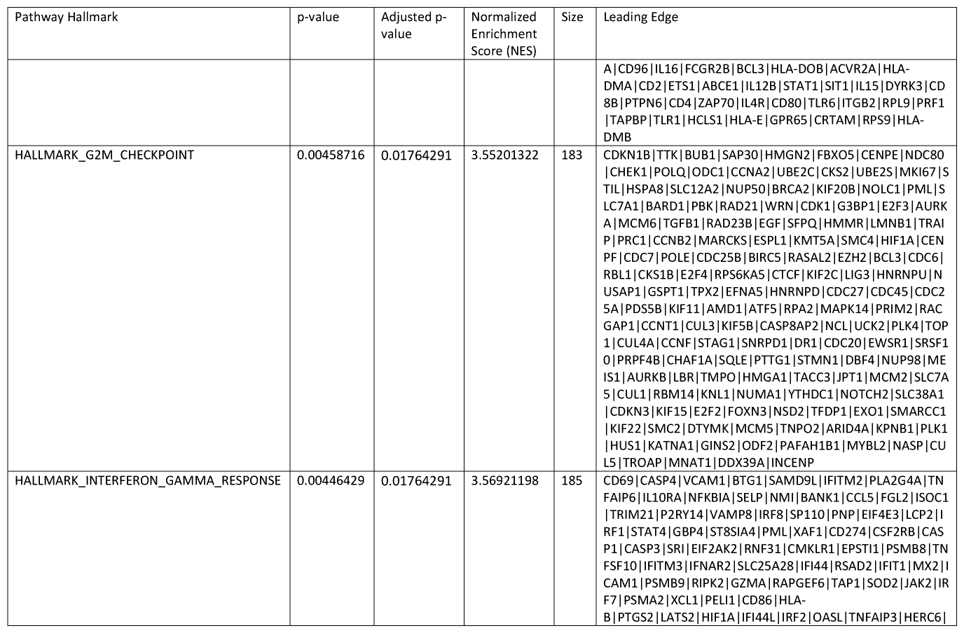

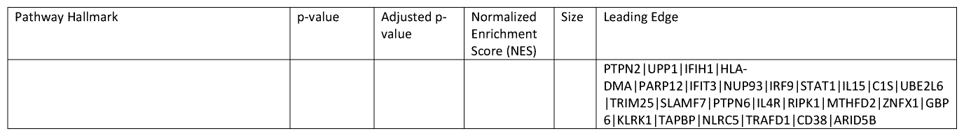

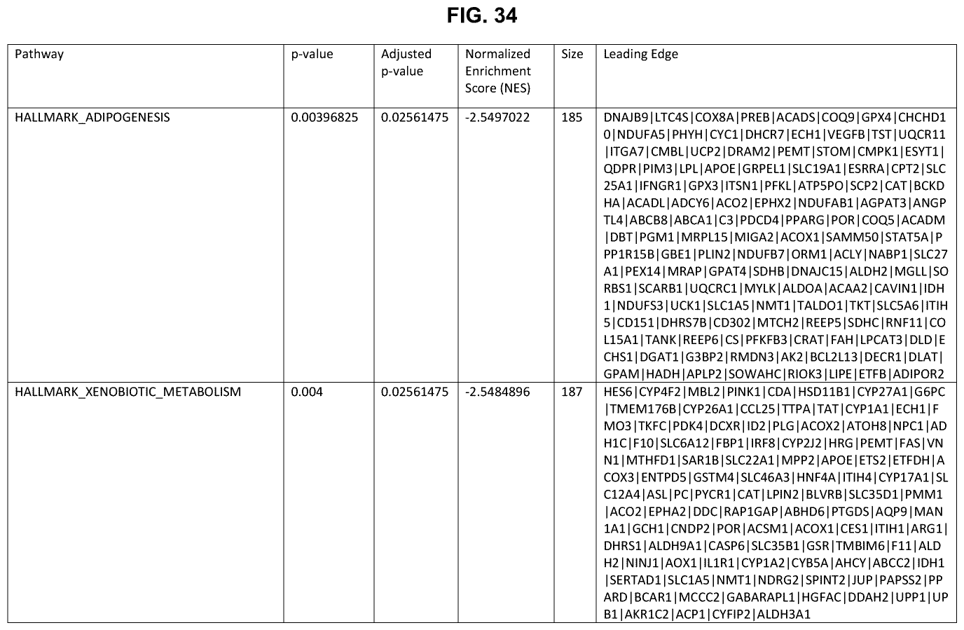

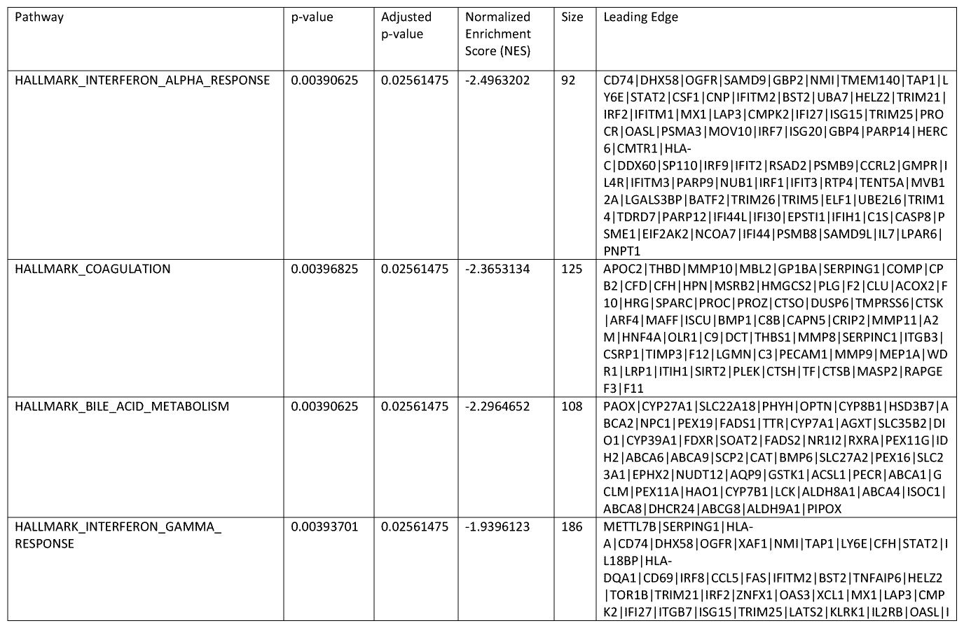

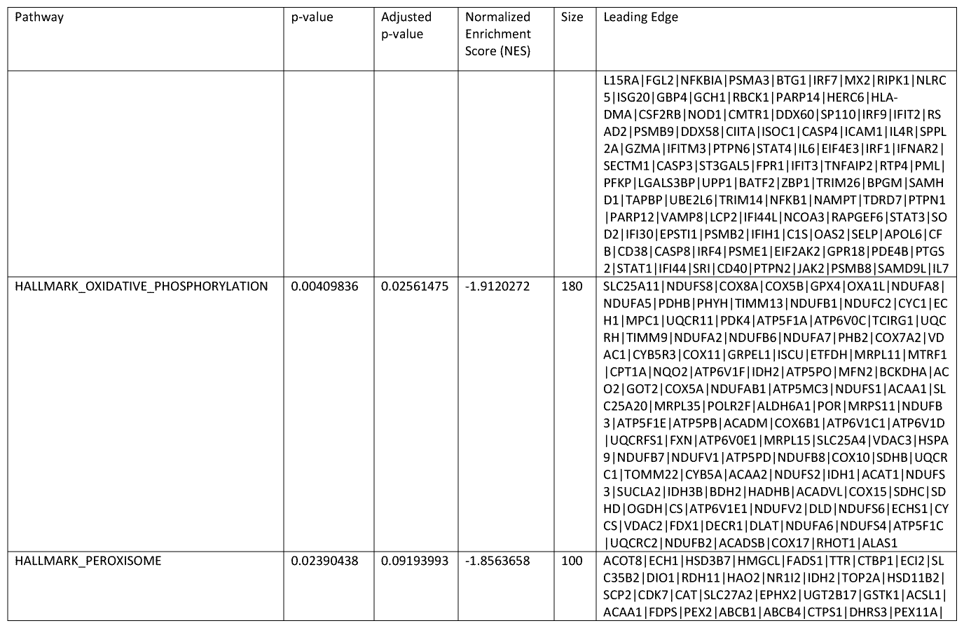

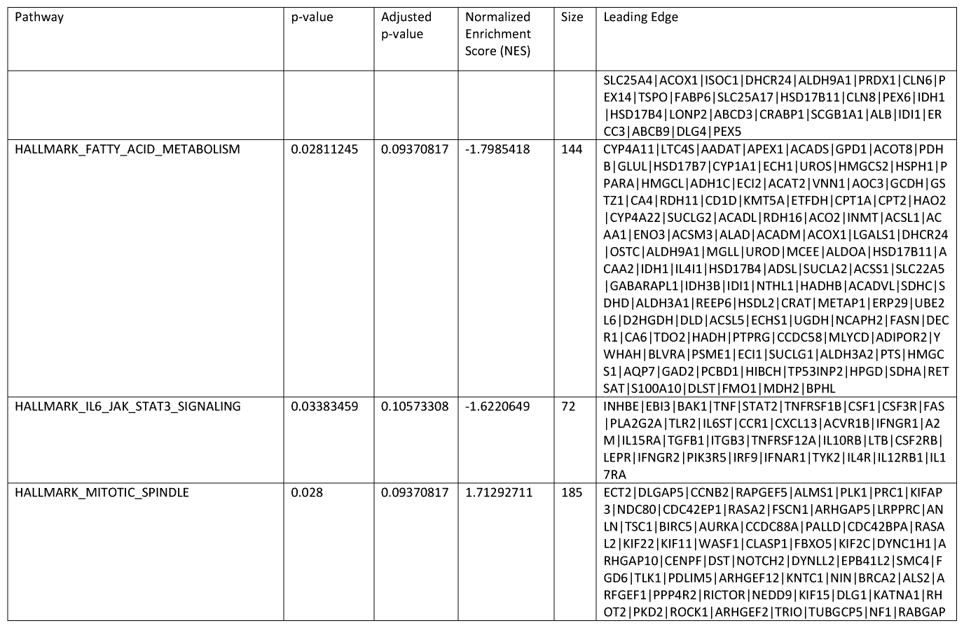

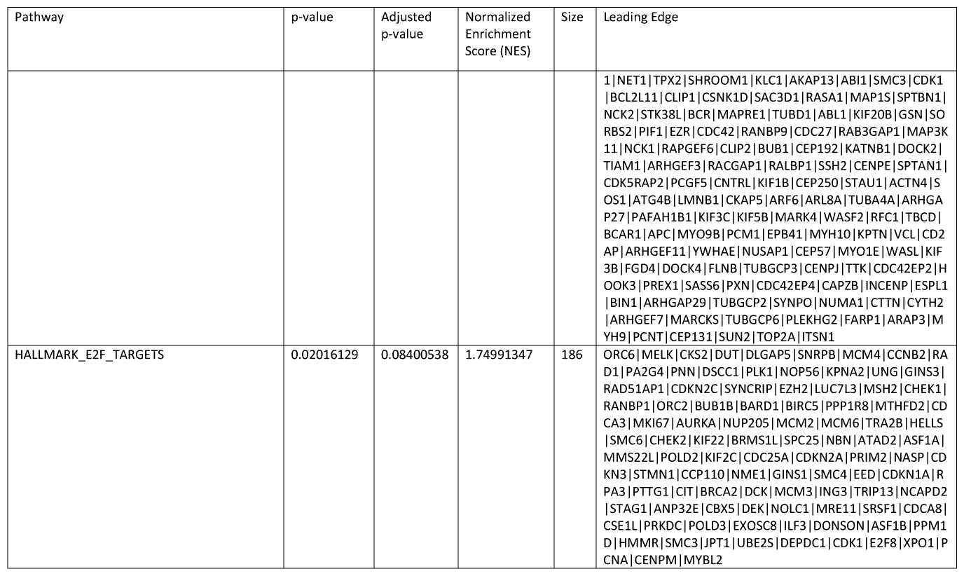

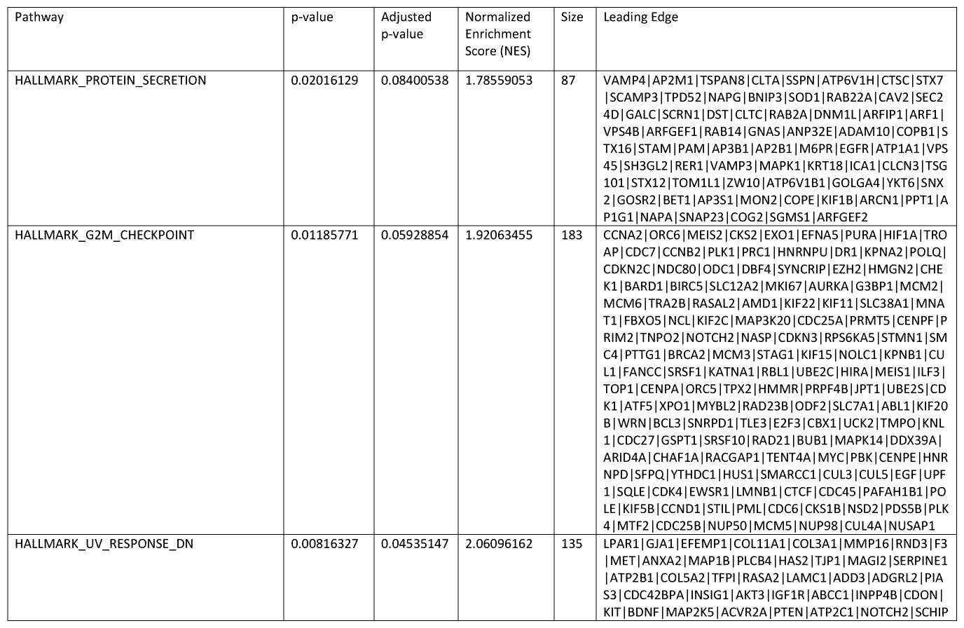

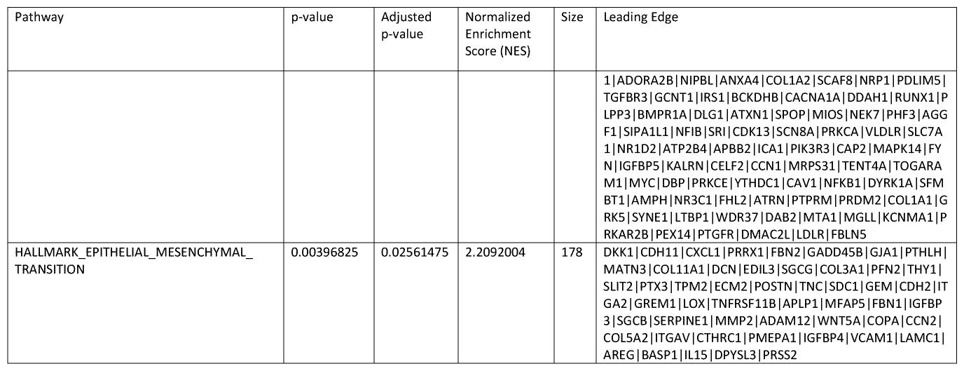

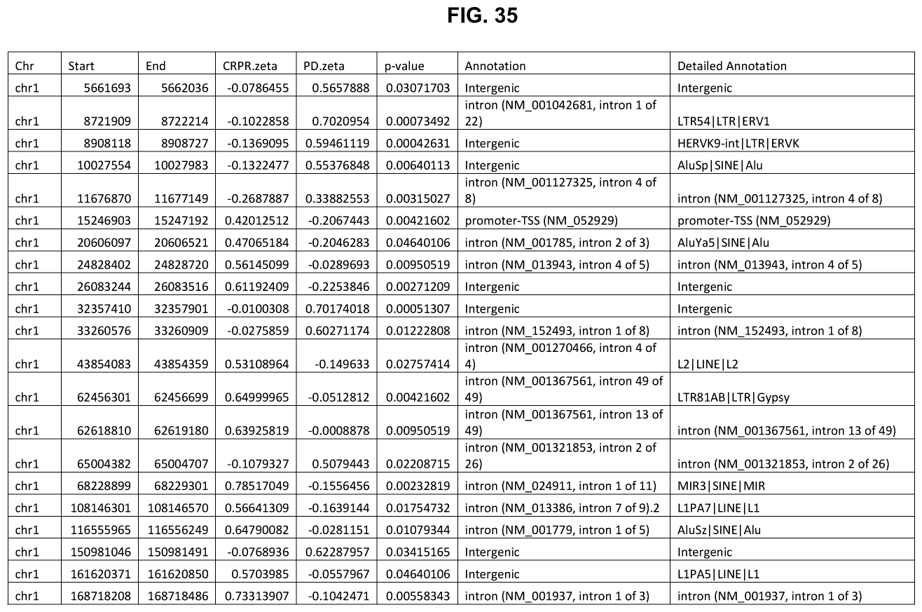

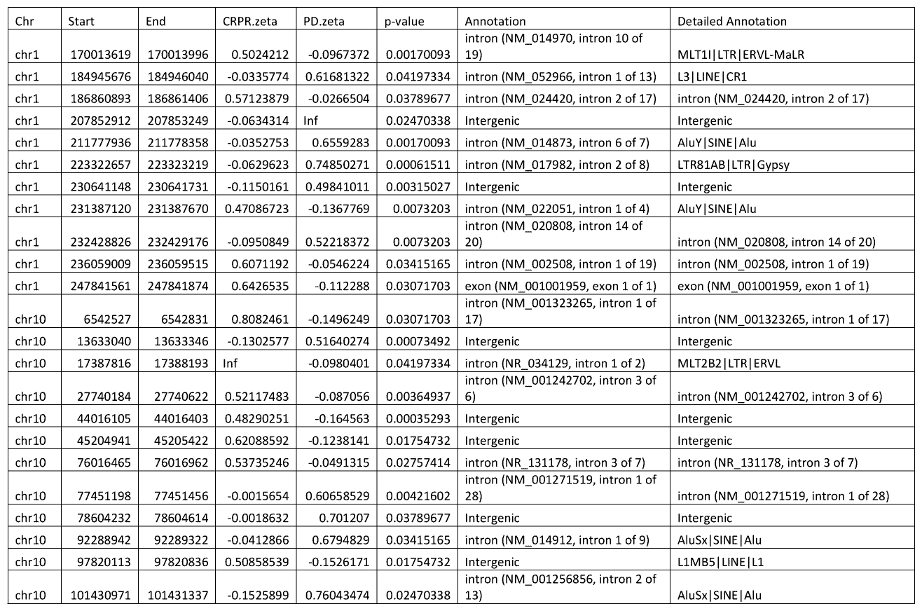

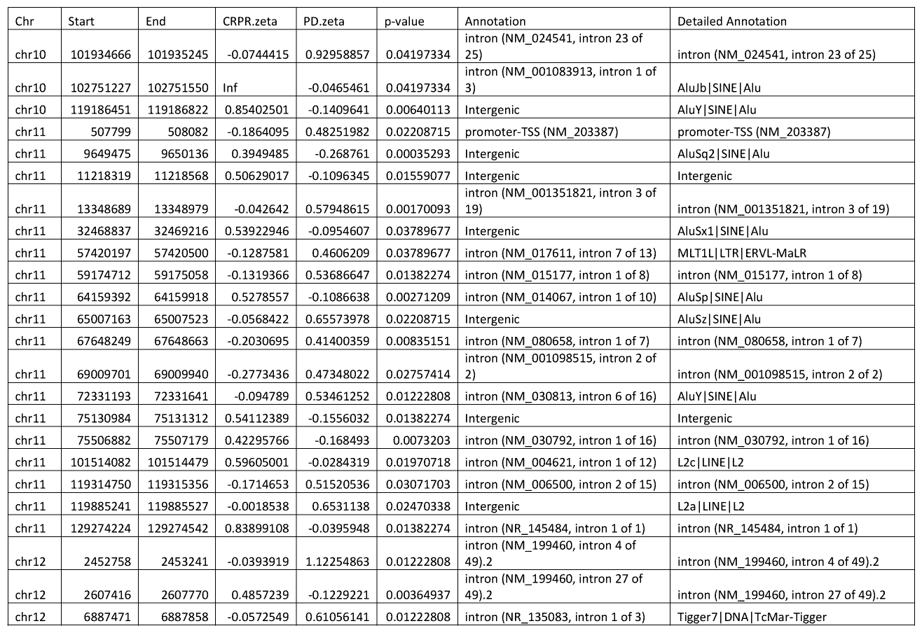

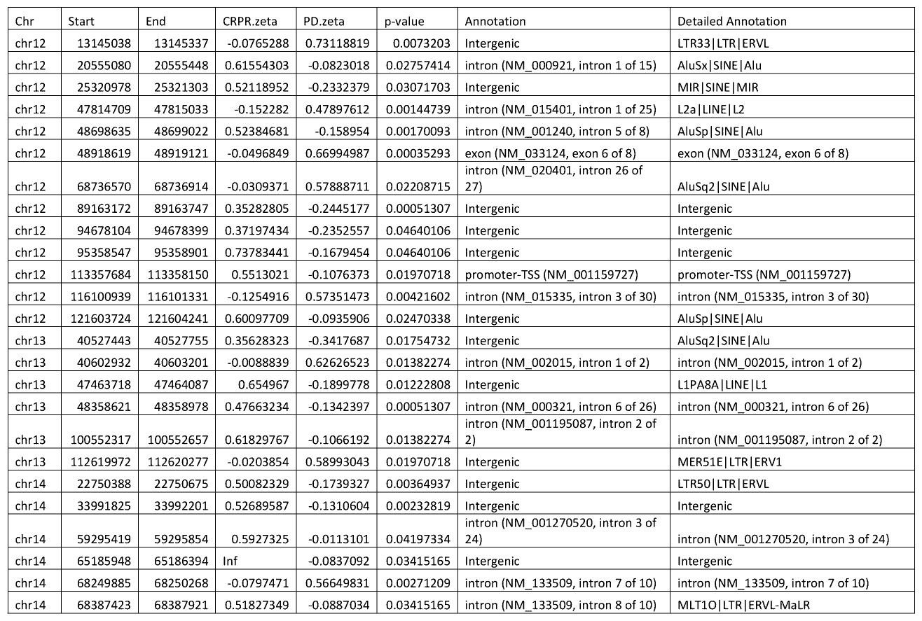

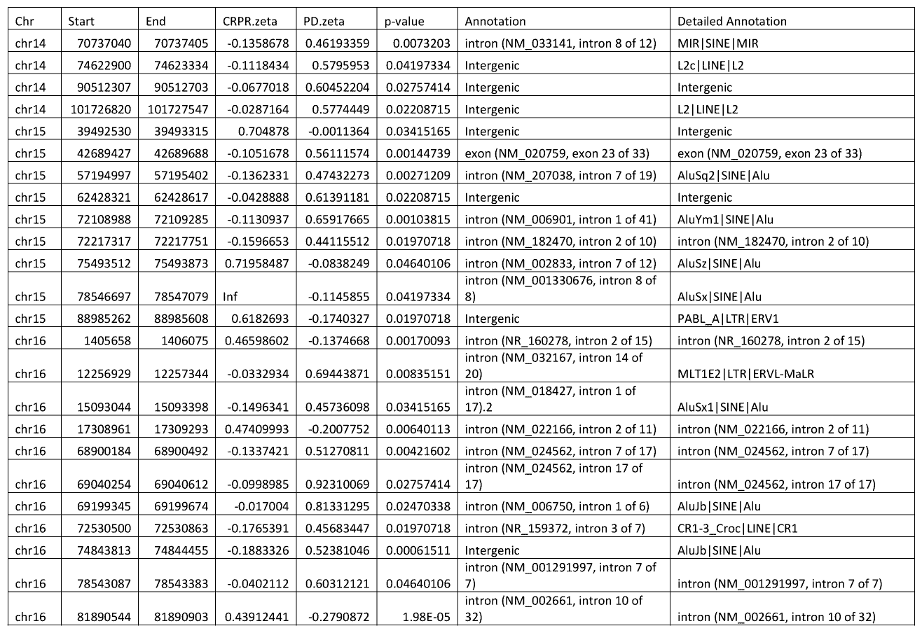

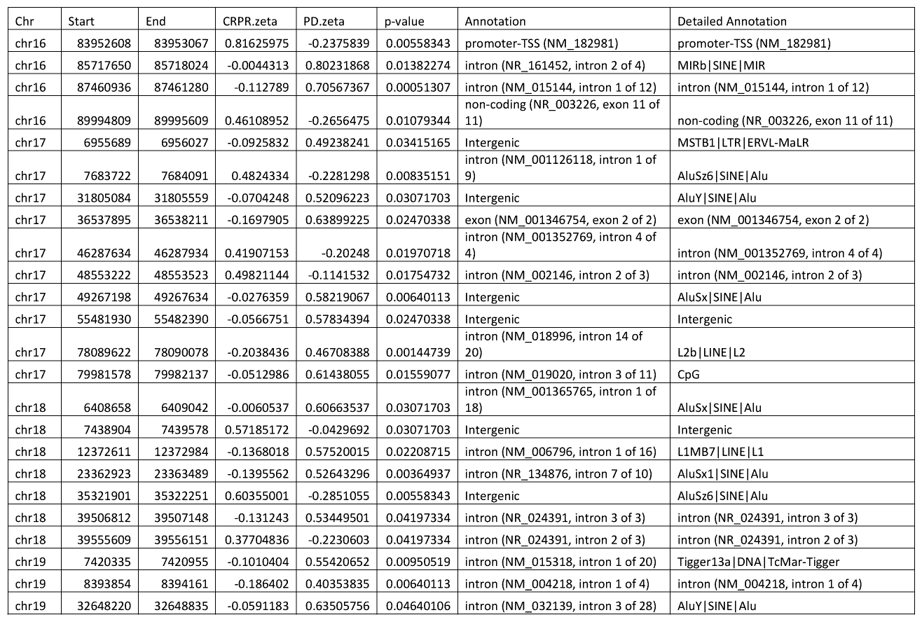

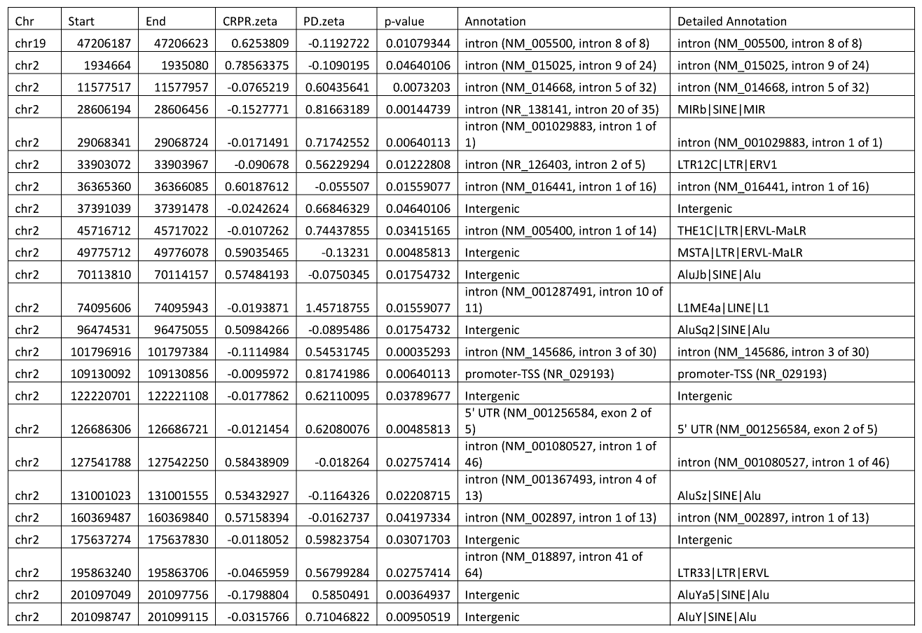

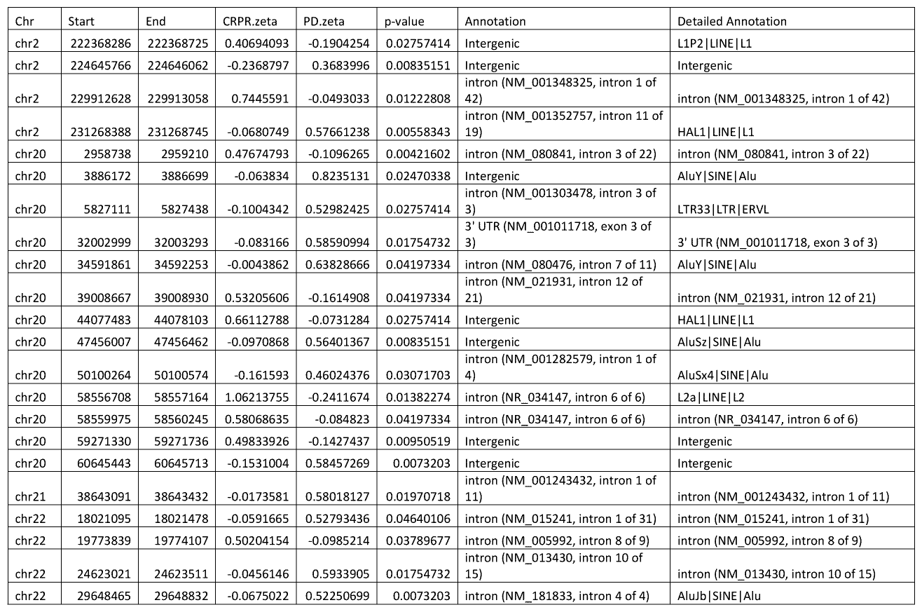

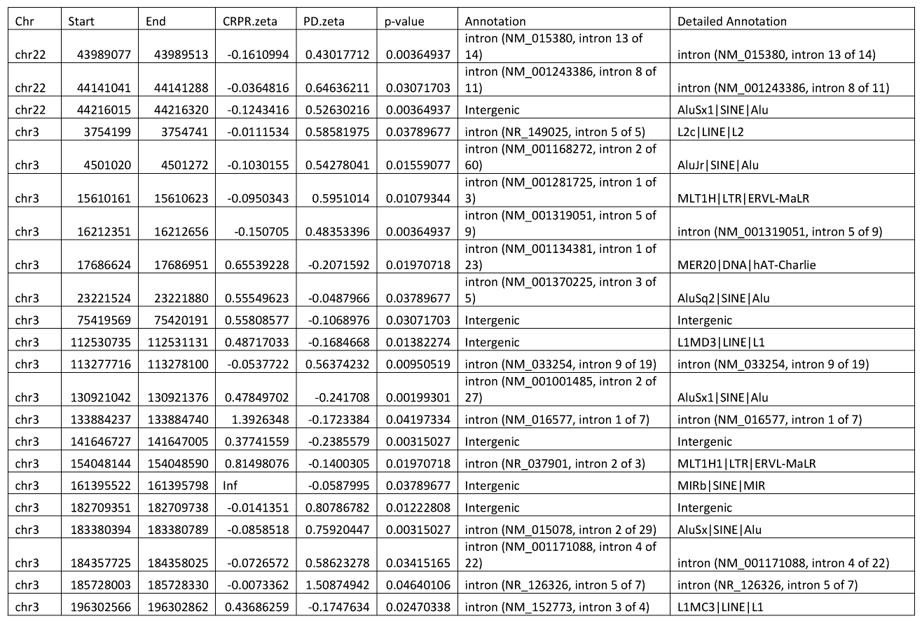

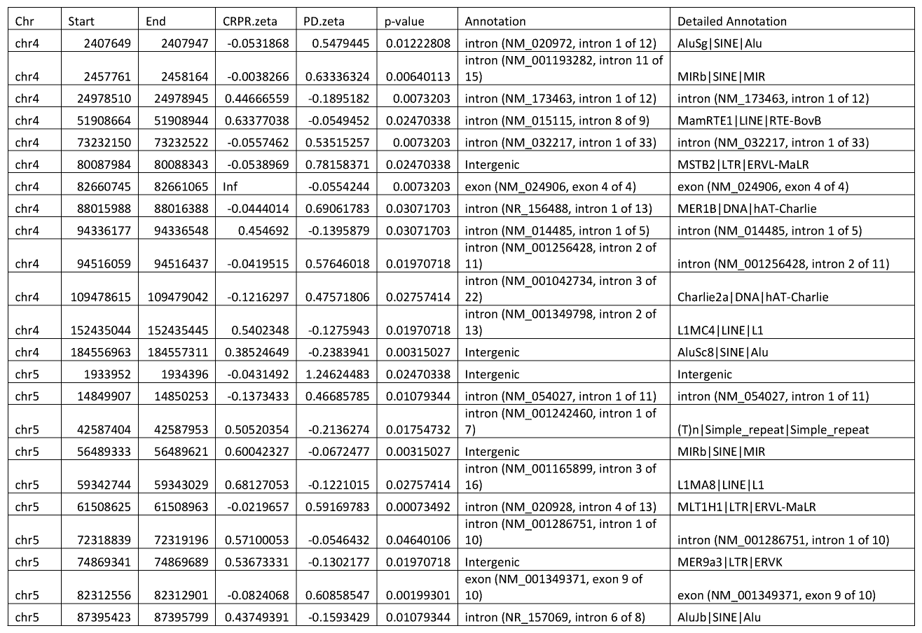

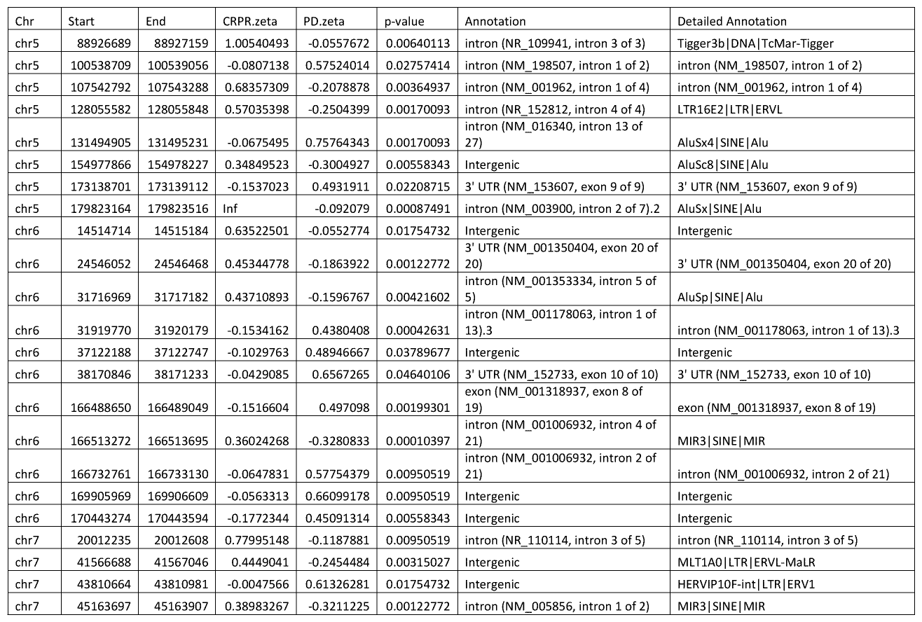

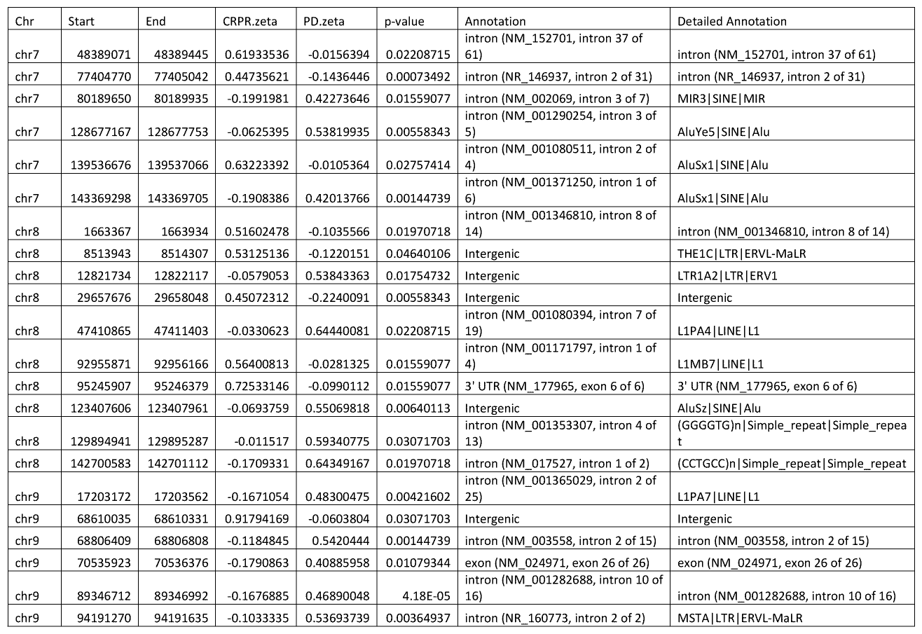

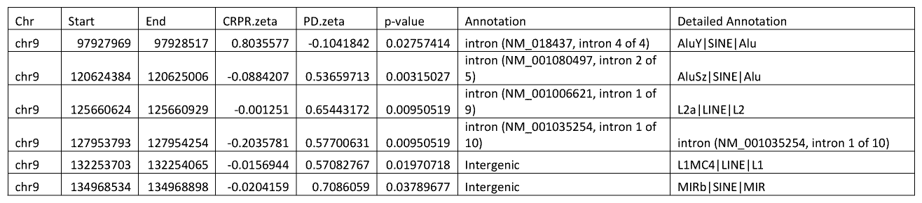

21. The method of claim 1, wherein the plurality of hydroxymethylation biomarker loci are selected from those in the tables of FIGS. 29-38.

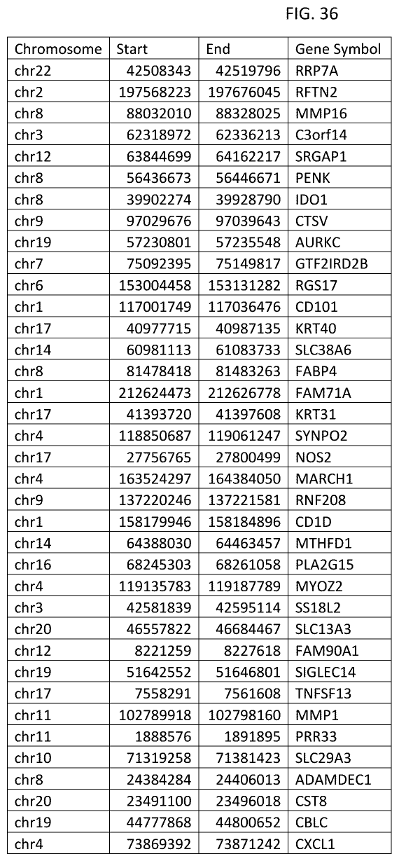

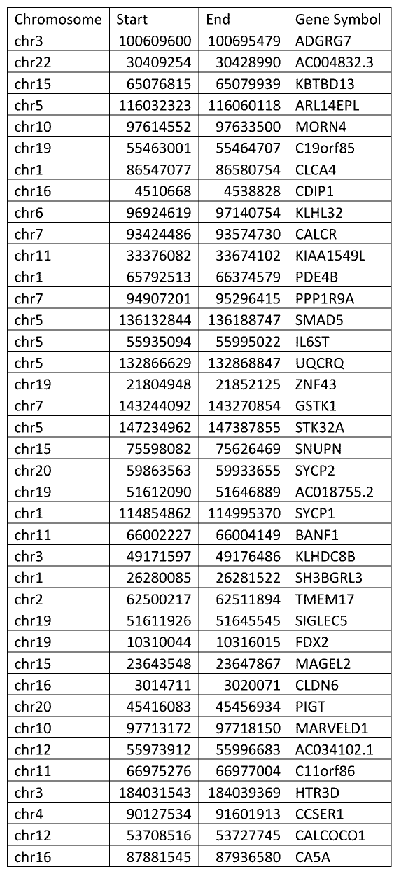

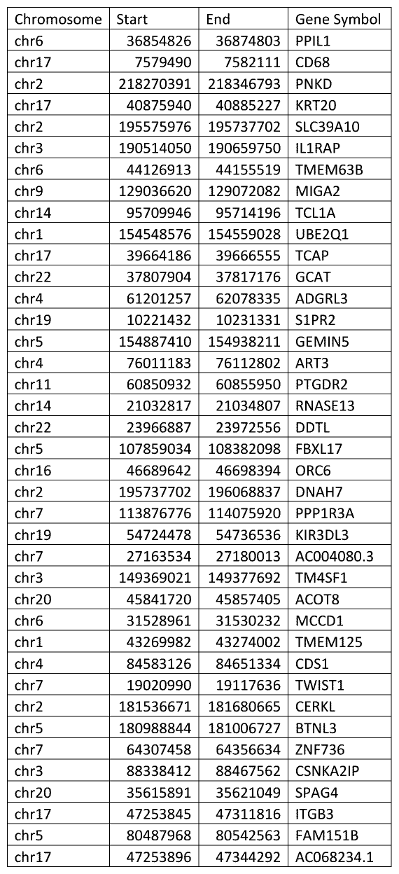

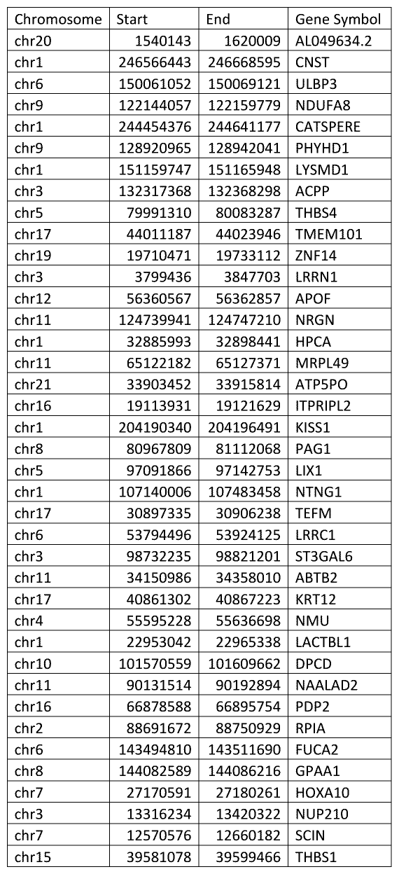

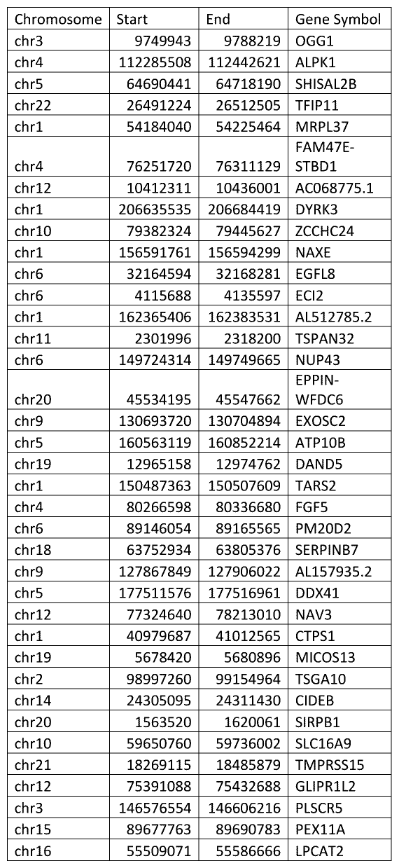

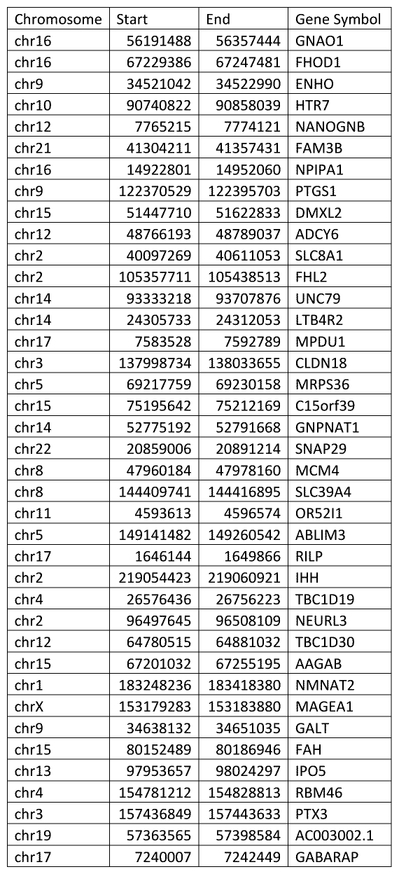

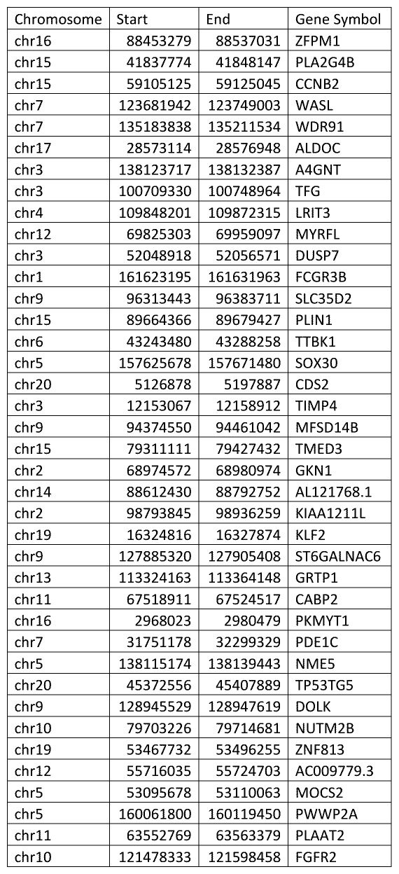

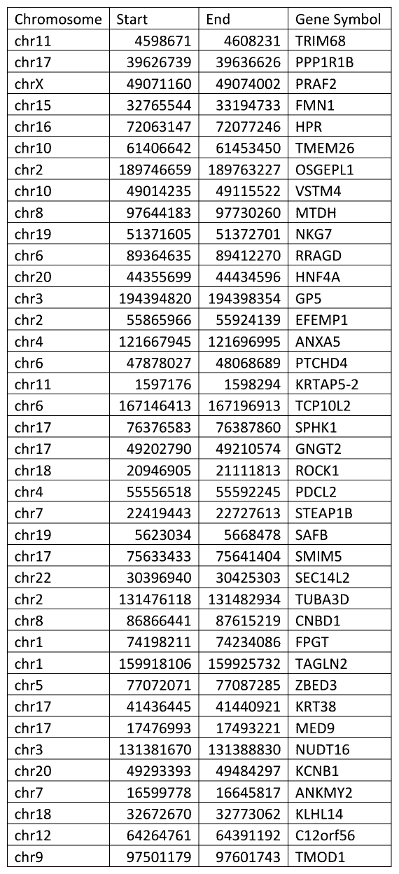

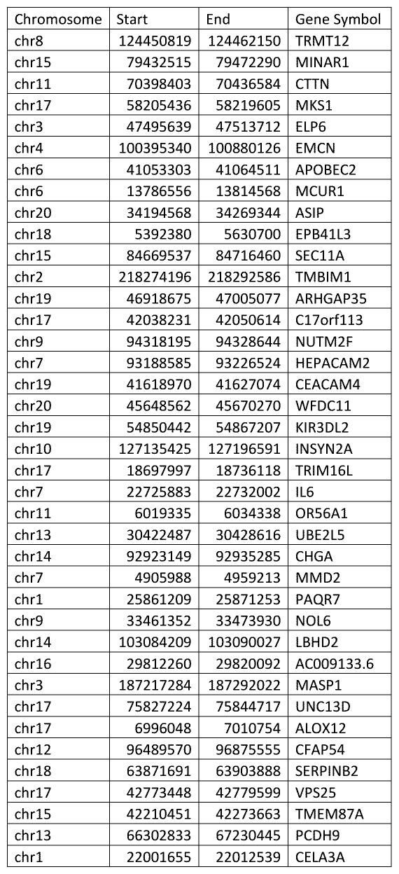

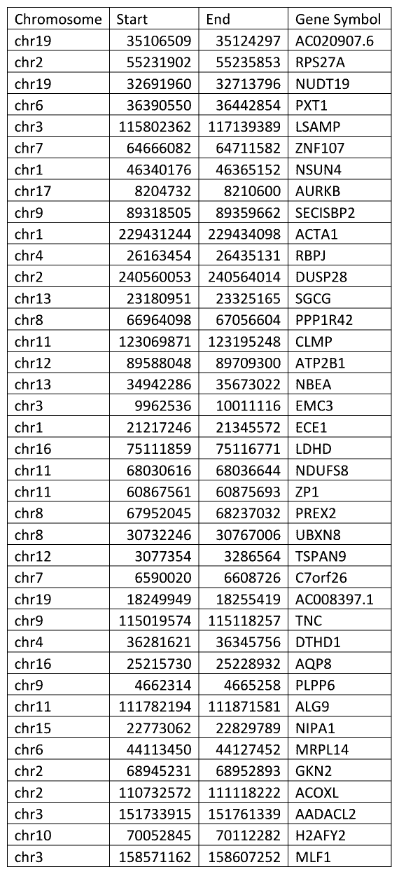

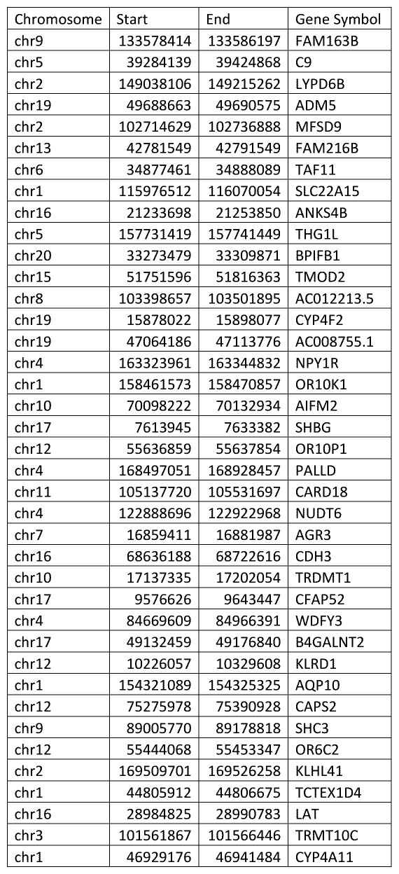

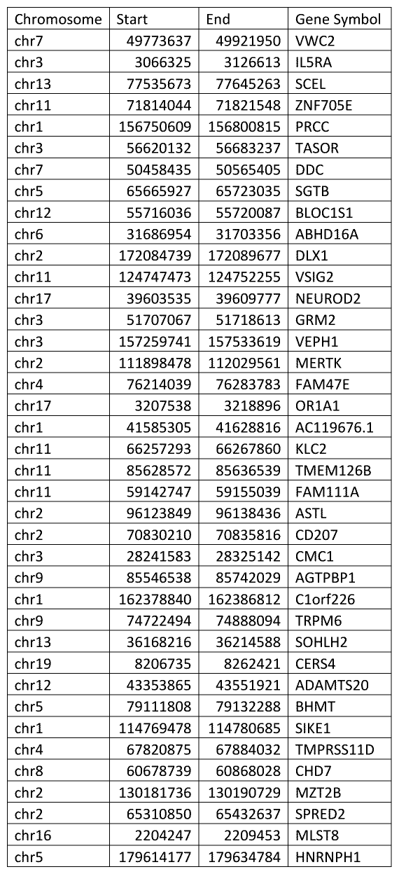

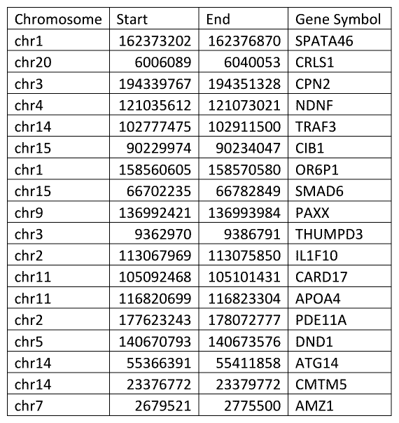

22. The method of claim 21, wherein the plurality of hydroxymethylation biomarker loci are selected from those in the tables of FIGS. 36-38.

23. A method for determining a likelihood that a lung cancer patient will respond to treatment with a selected lung cancer therapy, where the method comprises:
(a) obtaining a hydroxymethylation signature for a lung cancer patient by: (i) obtaining a cell-free DNA (cfDNA) sample from the patient, enriching for hydroxymethylated DNA in the sample, amplifying the hydroxymethylated DNA, and sequencing the amplified hydroxymethylated DNA in a manner that identifies 5-hydroxymethylcytosine (5hmC)-containing fragments or sites in the DNA;
(b) mapping the sequenced hydroxymethylated DNA to each of a plurality of hydroxymethylation biomarker loci in a reference hydroxymethylation profile comprising a composite of hydroxymethylation signatures for a population group of individuals who have at least one shared characteristic selected from having lung cancer and responding to a lung cancer therapy and having lung cancer and not responding to the lung cancer therapy;
(c) determining differences in extent and location between the patient hydroxymethylation signature and the reference hydroxymethylation profile at each locus; and
(d) using the extent and location of the differences, calculating a probability score representing the likelihood that the lung cancer patient will respond to treatment with a lung cancer therapy.

24. The method of claim 23, wherein each hydroxymethylation signature in the composite comprises a hydroxymethylation level at each of a plurality of hydroxymethylation biomarker loci.

25. The method of claim 24, wherein the plurality of hydroxymethylation biomarker loci in the reference hydroxymethylation profile are selected from those in the tables of FIGS. 29-38.

26. The method of claim 23, wherein each hydroxymethylation biomarker locus is selected as exhibiting an increase or decrease in hydroxymethylation in a manner that correlates with response to immunotherapy.

27. The method of claim 23, wherein each hydroxymethylation biomarker locus is selected as exhibiting an increase or decrease in hydroxymethylation in a manner that correlates with lung cancer tumor load.

28. The method of claim 23, wherein the probability score is calculated using a logistic regression analysis of the differences in hydroxymethylation level between the patient hydroxymethylation signature and the reference hydroxymethylation profile at each hydroxymethylation biomarker locus.

29. The method of claim 23, wherein each hydroxymethylation biomarker locus is selected as exhibiting differential hydroxymethylation as determined by a Wilcoxon rank-sum test with a p-value of less than 0.05 and a fold change of at least 1.5 between lung cancer patients who do not respond to the lung cancer therapy and lung cancer patients who do respond to the lung cancer therapy.

30. The method of claim 23, wherein (d) further comprises combining the probability score with an additional feature value for at least one additional feature type to characterize the likelihood that the patient will respond to the lung cancer therapy.

31. The method of claim 30, wherein the additional feature type comprises DNA fragment size distribution, copy number variation, cfDNA concentration, methylation profile, T-cell-inflamed gene expression profile, circulating tumor DNA count, serum CA19-9 level, serum CA125 level, LAG3 expression, IDO-1 expression, T-cell count, inflammation gene signature, myeloid-derived suppressor cell count, lymphocyte count, deficient mismatch repair, tumor mutational burden, presence or absence of germline mutations, a patient-specific clinical parameter, and combinations of any of the foregoing.

32. The method of claim 30, wherein the additional feature type comprises:
number of cfDNA fragments in each of at least two nonoverlapping size ranges;
copy number variation in the cfDNA sample;
concentration of cfDNA in the cfDNA sample;
a patient-specific clinical parameter; and
combinations of any of the foregoing.

33. The method of claim 32, wherein the patient-specific clinical parameter is selected from lesion size; lesion grade; lesion stage; lesion location; patient age; patient weight; patient gender; patient ethnicity; cigarette smoking status; and exposure or lack of exposure to a known carcinogen.

34. The method of claim 30, wherein the combining comprises an ensemble analysis.

35. The method of claim 34, wherein the combining comprises a stacked ensemble analysis.

36. The method of claim 23, wherein responding to immunotherapy comprises exhibiting a partial response, a complete response, or stable disease as defined in RECIST guidelines 1.1.

37. The method of claim 23, wherein the lung cancer is non-small cell lung cancer.

38. The method of claim 23, wherein the lung cancer is selected from adenocarcinomas, squamous cell carcinomas, small-cell lung carcinomas, adenosquamous carcinomas, carcinoid tumors, bronchial gland carcinomas, and sarcomatoid carcinomas.

39. The method of claim 23, wherein the lung cancer therapy is an immunotherapy.

40. (canceled)

41. (canceled)

42. A method for identifying differentially hydroxymethylated sites for use as hydroxymethylation biomarkers in evaluating a lung cancer patient's response to a therapy, wherein the method comprises:
(a) obtaining cfDNA from each of a plurality of lung cancer patients who are known responders or known nonresponders to the therapy;
(b) determining a baseline count T₀ in CPM at each of a plurality of candidate hydroxymethylation biomarker loci in the cfDNA obtained from each of the patients;
(c) determining a later count T_{R} in CPM after beginning the therapy and confirming response or nonresponse to the therapy, wherein T_{R} is determined for each of the plurality of candidate hydroxymethylation biomarker loci in the cfDNA obtained from each of the patients;
(d) selecting as hydroxymethylation biomarker loci those candidate hydroxymethylation biomarker loci exhibiting a threshold p-value of less than 0.05 and a difference z of at least 1.5, wherein
$$z = \{ {T}_{R} - {T}_{0} ) / {T}_{0} .$$

43. The method of claim 42, further including calculating a value for
log 2(T_{R}/T₀)
at each of the selected hydroxymethylated biomarker loci, and identifying the calculated values as xᵢ at each locus i or yⱼ at each locus j, wherein the xᵢ and yⱼ are positively and negatively correlated with treatment response, respectively.

44. A method for determining whether a lung cancer patient is responding to a lung cancer therapy, the method comprising:
(a) in a cfDNA sample obtained from the patient, determining a baseline count T₀ at each of the hydroxymethylation biomarker loci selected in claim 42;
(b) in a later cfDNA sample obtained from the patient, determining a later count T₀ at each of the hydroxymethylation biomarker loci selected in claim 42 after beginning the therapy;
(c) calculating a value for
log 2(T_{Q}/T₀)
at each of the selected hydroxymethylated biomarker loci, and identifying the calculated values as xᵢ at each locus i or yⱼ at each locus j, wherein the xᵢ and yⱼ are positively and negatively correlated with treatment response, respectively;
(e) calculating a 5hmC molecular response score (MR_{5hmc}) for the patient using the equation
$${MR}_{5 hmC} = {m}_{x} - {m}_{y}$$
wherein mx is the mean of the xᵢ over i loci and my is the mean of the yⱼ over j loci; and
(f) determining that the patient is responding to the therapy when the 5hmC molecular response score is positive.

45. The methods of claim 44, wherein the lung cancer therapy is immunotherapy.
